SEQUENCE LISTING (41 sequences)

SEQ ID NO: 1 - protein, 452 aa, Artificial Sequence
  R2M13-03 parental LALAPG- Heavy chain
EVQLLQSGAEVKKPGSSVKVSCKASGGTFTYRYLHWVRQAPGQGLEWMGGIIPIFGTGNYAQKFQGRVTITADESTSTAYMELSSLRSEDTAVYYCASSMVRVPYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 339 aa, Artificial Sequence
  R2M13-03 parental LALAPG- light chain
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 452 aa, Artificial Sequence; identical to SEQ ID NO: 1
  R2M13-03 humanized LALAPG- heavy chain

SEQ ID NO: 4 - protein, 336 aa, Artificial Sequence
  R2M13-03 humanized LALAPG- light chain
EVQLVESGGGLVQPGGSLRLSCASSANIQSIETLGWYRQAPGKQRELIANMRGGGYMKYADSLKGRFTMSTDNSKNTMYLQMNSLRAEDTAVYYCYVKLRDEDYVYRGQGTQVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 452 aa, Artificial Sequence
  R2M13-03 humanized N297G- heavy chain
EVQLLQSGAEVKKPGSSVKVSCKASGGTFTYRYLHWVRQAPGQGLEWMGGIIPIFGTGNYAQKFQGRVTITADESTSTAYMELSSLRSEDTAVYYCASSMVRVPYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 336 aa, Artificial Sequence; identical to SEQ ID NO: 4
  R2M13-03 humanized N297G- light chain

SEQ ID NO: 7 - protein, 452 aa, Artificial Sequence; identical to SEQ ID NO: 1
  R2M13-26 parental LALAPG (R2M13-26)-heavy chain

SEQ ID NO: 8 - protein, 338 aa, Artificial Sequence
  R2M13-26 parental LALAPG (R2M13-26)-light chain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 452 aa, Artificial Sequence; identical to SEQ ID NO: 1
  R2M13-26 humanized LALAPG (R2M13-h26)-heavy chain

SEQ ID NO: 10 - protein, 338 aa, Artificial Sequence
  R2M13-26 humanized LALAPG (R2M13-h26)-light chain
EVQLVESGGGLVQPGGSLRLSCAGSGRIFAIYDIAWYRQAPGKGREWVAMIRPVVTEIDYADSVKGRFTISRDNSKKTVYLQMNSLRAEDTAVYYCNAKRPWGSRDEYWGQGTTVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 452 aa, Artificial Sequence; identical to SEQ ID NO: 5
  R2M13-26 humanized N297G-heavy chain

SEQ ID NO: 12 - protein, 338 aa, Artificial Sequence; identical to SEQ ID NO: 10
  R2M13-26 humanized N297G-light chain

SEQ ID NO: 13 - protein, 452 aa, Artificial Sequence; identical to SEQ ID NO: 1
  R2M13-36 parental LALAPG-heavy chain

SEQ ID NO: 14 - protein, 330 aa, Artificial Sequence
  R2M13-36 parental LALAPG-light chain
QVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGSGSGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 452 aa, Artificial Sequence; identical to SEQ ID NO: 1
  R2M13-36 humanized LALAPG-heavy chain

SEQ ID NO: 16 - protein, 330 aa, Artificial Sequence
  R2M13-36 humanized LALAPG-light chain
EVQLVESGGGLVQPGGSLRLSCAASGRIFSIYDMGWFRQAPGKEREFVSGIRWSGGTSYADSVKGRFTISKDNSKNTIYLQMNSLRAEDTAVYYCGSRGYWGQGTLVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 452 aa, Artificial Sequence; identical to SEQ ID NO: 5
  R2M13-36 humanized N297G-heavy chain

SEQ ID NO: 18 - protein, 330 aa, Artificial Sequence; identical to SEQ ID NO: 16
  R2M13-36 humanized N297G-light chain

SEQ ID NO: 19 - protein, 119 aa, Artificial Sequence
  VHH26_H1
EVQLLESGGGLVQPGGSLRLSCAASGRIFAIYDIAWVRQAPGKGLEWVSMIRPVVTEIDYADSVKGRFTISRDNSKNTLYLQMNSLRAEDTAVYYCAKKRPWGSRDEYWGQGTTVTVSS

SEQ ID NO: 20 - protein, 119 aa, Artificial Sequence
  VHH26_H2
EVQLVESGGGLVQPGGSLRLSCAGSGRIFAIYDIAWYRQAPGKGLEWVAMIRPVVTEIDYADSVKGRFTISRDNSKNTVYLQMNSLRAEDTAVYYCNAKRPWGSRDEYWGQGTTVTVSS

SEQ ID NO: 21 - protein, 119 aa, Artificial Sequence
  VHH26_H3
EVQLVESGGGLVQPGGSLRLSCAGSGRIFAIYDIAWYRQAPGKGREWVAMIRPVVTEIDYADSVKGRFTISRDNSKNTVYLQMNSLRAEDTAVYYCNAKRPWGSRDEYWGQGTTVTVSS

SEQ ID NO: 22 - protein, 119 aa, Artificial Sequence
  VHH26_H4
EVQLVESGGGLVQPGGSLRLSCAGSGRIFAIYDIAWYRQAPGKGRELVAMIRPVVTEIDYADSVKGRFTISRDNSKNTVYLQMNSLRAEDTAVYYCNAKRPWGSRDEYWGQGTTVTVSS

SEQ ID NO: 23 - protein, 119 aa, Artificial Sequence
  VHH26_H5
EVQLVESGGGLVQPGGSLRLSCAGSGRIFAIYDIAWYRQAPGKGREWVAMIRPVVTEIDYADSVKGRFTISRDNSKKTVYLQMNSLRAEDTAVYYCNAKRPWGSRDEYWGQGTTVTVSS

SEQ ID NO: 24 - protein, 119 aa, Artificial Sequence
  VHH26_H6
EVQLVESGGGLVQPGGSLRLSCAGSGRIFAIYDIAWYRQAPGKGRELVAMIRPVVTEIDYADSVKGRFTISRDNSKKTVYLQMNSLRAEDTAVYYCNAKRPWGSRDEYWGQGTTVTVSS

SEQ ID NO: 25 - protein, 119 aa, Artificial Sequence
  VHH26 (parental)
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSS

SEQ ID NO: 26 - protein, 60 aa, Artificial Sequence
  VHH26_ parental-align 1
DVQLVESGGGLVQAGGSLRLACAGSGRIFAIYDIAWYRHPPGNQRELVAMIRPVVTEIDY

SEQ ID NO: 27 - protein, 60 aa, Artificial Sequence
  IGHV3-23_IGHJ6-align 1
EVQLLESGGGLVQPGGSLRLSCAASGFTFSSYAMSWVRQAPGKGLEWVSAISGSGGSTYY

SEQ ID NO: 28 - protein, 60 aa, Artificial Sequence
  VHH26-H1-align 1
EVQLLESGGGLVQPGGSLRLSCAASGRIFAIYDIAWVRQAPGKGLEWVSMIRPVVTEIDY

SEQ ID NO: 29 - protein, 60 aa, Artificial Sequence
  VHH26-H2-align 1
EVQLVESGGGLVQPGGSLRLSCAGSGRIFAIYDIAWYRQAPGKGLEWVAMIRPVVTEIDY

SEQ ID NO: 30 - protein, 60 aa, Artificial Sequence
  VHH26-H3-align 1
EVQLVESGGGLVQPGGSLRLSCAGSGRIFAIYDIAWYRQAPGKGREWVAMIRPVVTEIDY

SEQ ID NO: 31 - protein, 60 aa, Artificial Sequence
  VHH26-H4-align 1
EVQLVESGGGLVQPGGSLRLSCAGSGRIFAIYDIAWYRQAPGKGRELVAMIRPVVTEIDY

SEQ ID NO: 32 - protein, 60 aa, Artificial Sequence; identical to SEQ ID NO: 30
  VHH26-H5-align 1

SEQ ID NO: 33 - protein, 60 aa, Artificial Sequence; identical to SEQ ID NO: 31
  VHH26-H6-align 1

SEQ ID NO: 34 - protein, 58 aa, Artificial Sequence
  VHH26_ parental-align 2
ADSVKGRFTISRNNAMKTVYLQMNNLKPEDTAVYYCNAKRPWGSRDEYWGQGTQVTSS

SEQ ID NO: 35 - protein, 57 aa, Artificial Sequence
  IGHV3-23_IGHJ6-align 2
ADSVKGRFTISRDNSKNTLYLQMNSLRAEDTAVYYCAKYYYYYGMDVWGQTTVTVSS

SEQ ID NO: 36 - protein, 59 aa, Artificial Sequence
  VHH26-H1-align 2
ADSVKGRFTISRDNSKNTLYLQMNSLRAEDTAVYYCAKKRPWGSRDEYWGQGTTVTVSS

SEQ ID NO: 37 - protein, 59 aa, Artificial Sequence
  VHH26-H2-align 2
ADSVKGRFTISRDNSKNTVYLQMNSLRAEDTAVYYCNAKRPWGSRDEYWGQGTTVTVSS

SEQ ID NO: 38 - protein, 59 aa, Artificial Sequence; identical to SEQ ID NO: 37
  VHH26-H3-align 2

SEQ ID NO: 39 - protein, 59 aa, Artificial Sequence; identical to SEQ ID NO: 37
  VHH26-H4-align 2

SEQ ID NO: 40 - protein, 59 aa, Artificial Sequence
  VHH26-H5-align 2
ADSVKGRFTISRDNSKKTVYLQMNSLRAEDTAVYYCNAKRPWGSRDEYWGQGTTVTVSS

SEQ ID NO: 41 - protein, 59 aa, Artificial Sequence; identical to SEQ ID NO: 40
  VHH26-H6-align 2

(19) United States
(12) Patent Application Publication
(10) Pub. No.: US 2024/0150473 A1
(43) Pub. Date: May 9, 2024

(54) MODULATION OF WNT SIGNALING IN GASTROINTESTINAL DISORDERS

(71) Applicant: Surrozen Operating, Inc., South San Francisco, CA (US)

(72) Inventors: Russell FLETCHER, South San Francisco, CA (US); Sungjin LEE, Palo Alto, CA (US); Yang LI, Mountain View, CA (US); Chenggang LU, Foster City, CA (US); Parthasarathy SAMPATHKUMAR, Foster City, CA (US); Geertrui VANHOVE, South San Francisco, CA (US); Wen-Chen YEH, Belmont, CA (US); Liqin XIE, Elmsford, NY (US); Leonard PRESTA, San Francisco, CA (US)

(21) Appl. No.: 18/281,253

(22) PCT Filed: Mar. 9, 2022
(86) PCT No.: PCT/US2022/019614

§ 371 Date: Sep. 8, 2023

Related U.S. Application Data
(60) Provisional application No. 63/247,151, filed on Sep. 22, 2021.
(60) Provisional application No. 63/190,535, filed on May 19, 2021.
(60) Provisional application No. 63/159,010, filed on Mar. 10, 2021.

(51) Int. Cl. C07K 16/28; A01N 1/02; A61P 1/00; A61P 29/00; C12N 5/071

(52) U.S. Cl. CPC C07K 16/2863; A01N 1/0226; A61P 1/00; A61P 29/00; C07K 16/28; C12N 5/0679; A61K 2039/505; C07K 2317/22; C07K 2317/24; C07K 2317/31; C07K 2317/569; C07K 2317/75

(57) ABSTRACT

The present disclosure provides engineered WNT agonists and methods of treating gastrointestinal disorders with modulators of the WNT signaling pathway.

### RELATED APPLICATIONS

This application claims priority to U.S. Provisional Patent Application Ser. No. 63/159,010, filed Mar. 10, 2021, U.S. Provisional Patent Application Ser. No. 63/190,535, filed May 19, 2021, and U.S. Provisional Patent Application Ser. No. 63/247,151, filed Sep. 22, 2021, which are incorporated herein by reference in their entireties.

### SEQUENCE LISTING

This application is being filed electronically via EFS-Web and includes an electronically submitted sequence listing in .txt format. The .txt file contains a sequence listing entitled SRZN_020_03WO_ST25.txt created on Mar. 7, 2022 and having a size of 80 kilobytes. The sequence listing contained in this .txt file is part of the specification and is incorporated herein by reference in its entirety.

### FIELD OF THE INVENTION

The disclosure provides WNT signal modulators as a treatment for gastrointestinal disorders, in particular, inflammatory bowel diseases.

### BACKGROUND

WNT proteins form a family of highly conserved secreted signaling molecules that regulate cell-to-cell interactions during embryogenesis. WNT genes and WNT signaling are also implicated in cancer. Insights into the mechanisms of WNT action have emerged from several systems: genetics in Drosophila and Caenorhabditis elegans; biochemistry in cell culture and ectopic gene expression in Xenopus embryos. Many WNT genes in the mouse have been mutated, leading to very specific developmental defects. As currently understood, WNT proteins bind to receptors of the Frizzled family on the cell surface. Through several cytoplasmic relay components, the signal is transduced to beta-catenin, which then enters the nucleus and forms a complex with TCF to activate transcription of WNT target genes. Expression of WNT proteins varies, but is often associated with the developmental process, for example in embryonic and fetal tissues.

The exploration of physiologic functions of WNT proteins in adult organisms has been hampered by functional redundancy and the necessity for conditional inactivation strategies. Dickkopf-1 (Dkk1) has been recently identified as the founding member of a family of secreted proteins that potently antagonize WNT signaling (see Glinka et al. (1998) Nature 391:357-62; Fedi et al. (1999) J Biol Chem 274:19465-72; and Bafico et al. (2001) Nat Cell Biol 3:683-6). Dkk1 associates with both the WNT co-receptors, LRP5 and LRP6, and the transmembrane protein Kremen, with the resultant ternary complex engendering rapid LRP6 internalization and impairment of WNT signaling through the absence of functional Frizzled/LRP6 WNT receptor complexes (see, e.g., Mao et al. (2001) Nature 411:321-5; Semenov et al. (2001) Curr Biol 11:951-61; and Mao et al. (2002) Nature 417:664-7).

Transgenic mice that have a knock-out of the Tcf locus show a loss of proliferative stem cell compartments in the small intestine during late embryogenesis. However, the knockout is lethal, and so has not been studied in adults. In chimeric transgenic mice that allow analysis of adults, expression of constitutively active NH2-truncated β-catenin stimulated proliferation in small intestine crypts, although either NH2-truncated β-catenin or Lef-1/β-catenin fusions induced increased crypt apoptosis as well. Because diverse factors regulate β-catenin/Lef/Tcf-dependent transcription, including non-Frizzled GPCRs and PTEN/PI-3-kinase, the cause of intestinal stem cell defect is not known.

The adult intestinal epithelium is characterized by continuous replacement of epithelial cells through a stereo-typed cycle of cell division, differentiation, migration and exfoliation occurring during a 5-7 day crypt-villus transit time. The putative growth factors regulating proliferation within the adult intestinal stem cell niche have not yet been fully identified, although studies have implicated the cell-intrinsic action of β-catenin/Lef/Tcf signaling within the proliferative crypt compartment.

A number of pathological conditions affect the cells of the intestines. Inflammatory bowel disease (IBD) can involve either or both the small and large bowel. Crohn's disease and ulcerative colitis are the best-known forms of IBD, and both fall into the category of “idiopathic” inflammatory bowel disease because the etiology for them is unknown. “Active” IBD is characterized by acute inflammation. “Chronic” IBD is characterized by architectural changes of crypt distortion and scarring. Crypt abscesses can occur in many forms of IBD.

Crohn's disease can involve any part of the GI tract, but most frequently involves the distal small bowel and colon. Inflammation is typically transmural and can produce anything from a small ulcer over a lymphoid follicle (aphthoid ulcer) to a deep fissuring ulcer to transmural scarring and chronic inflammation. One third of cases have granulomas, and extracolonic sites such as lymph nodes, liver, and joints may also have granulomas. The transmural inflammation leads to the development of fistulas between loops of bowel and other structures. Inflammation is typically segmental with uninvolved bowel separating areas of involved bowel. The etiology is unknown, though infectious and immunologic mechanisms have been proposed.

Ulcerative colitis (UC) involves the colon as a diffuse mucosal disease with distal predominance. The rectum is virtually always involved, and additional portions of colon may be involved extending proximally from the rectum in a continuous pattern. The etiology for UC is unknown. Patients with prolonged UC are at increased risk for developing colon cancer. Patients with UC are also at risk for development of liver diseases including sclerosing cholangitis and bile duct carcinoma. Currently, all therapeutics in the clinic and most in development for treatment of UC focus on reducing inflammation and do not directly induce epithelial healing, highlighting the unmet need for therapeutic agents that promote epithelial repair.

Developing pharmacologic agents for the regulation of intestinal epithelium growth is of great interest for clinical purposes. However, exploration of WNT agonists as pharmacological agents has been hampered, in part, by the fact that they are not naturally soluble, diffusible molecules. The present disclosure provides methods and compositions to specifically modulate WNT signaling through particular FZD receptors using engineered soluble WNT agonists. Such engineered WNT agonists may achieve, for example, epithelial-specific transient Wnt signaling activation, which drives robust epithelial regeneration and barrier restoration, ultimately leading to a reduction in inflammation and amelioration of colitis.

### SUMMARY OF THE INVENTION

In various aspects, the present disclosure provides engineered WNT agonist and related pharmaceutical compositions and methods of use.

In one aspect, the disclosure includes an engineered WNT agonist comprising: (a) one or more binding domains that bind to one or more FZD; and (b) one or more binding domains that bind to LRP5, LRP6, or both LRP5 and LRP6, wherein the engineered WNT agonist comprises a polypeptide sequence having at least 90%, at least 95%, at least 98%, or at least 99% sequence identity to any of SEQ ID NOs:1-18, or a polypeptide sequence disclosed in any one of SEQ ID NO:s 1-25, FIG. 2, FIG. 6, Table 1, or Table 3, or a functional fragment or variant thereof, e.g., a binding fragment thereof, e.g., a VHH domain, a variable domain of a heavy chain, or a variable domain of a light chain. In certain embodiments, the one or more binding domains that bind to one or more FZD bind to: i) FZD5; ii) FZD 8; iii) FZD 1; iv) FZD 2; v) FZD 7; vi) FZD 5 and FZD 8; vii) FZD 1, FZD 2, and FZD 7; viii) FZD 1, FZD 2, FZD 7, FZD 5 and FZD 8; ix) FZD4; x) FZD9; or xi) FZD10. In certain embodiments, the engineered WNT agonist comprises one or more (e.g., two) polypeptide sequence having at least 90%, at least 95%, %, at least 98%, or at least 99% sequence identity to any one of SEQ ID NOs:1-18 or 19-25 or a sequence disclosed in Table 3. In certain embodiments, the engineered WNT agonist comprises: (a) one or more (e.g., two) polypeptide sequence having least 90%, or at least 95% homology to SEQ ID NO: 1 and one or more (e.g., two) polypeptide sequence having at least 90%, or at least 95% homology to SEQ ID NO:2; (b) one or more (e.g., two) polypeptide sequence having least 90%, or at least 95% homology to SEQ ID NO: 3 and one or more (e.g., two) polypeptide sequence having at least 90%, or at least 95% homology to SEQ ID NO:4; (c) one or more (e.g., two) polypeptide sequence having at least 80%, at least 90%, or at least 95% homology to SEQ ID NO: 5 and one or more (e.g., two) polypeptide sequence having at least 80%, at least 90%, or at least 95% homology to SEQ ID NO:6; (d) one or more (e.g., two) polypeptide sequence having at least 90%, or at least 95% homology to SEQ ID NO: 7 and one or more (e.g., two) polypeptide sequence having at least 90%, or at least 95% homology to SEQ ID NO:8; (e) one or more (e.g., two) polypeptide sequence having at least 90%, or at least 95% homology to SEQ ID NO: 9 and one or more (e.g., two) polypeptide sequence having at least 90%, or at least 95% homology to SEQ ID NO: 10; (f) one or more (e.g., two) polypeptide sequence having at least 90%, or at least 95% homology to SEQ ID NO: 7 and one or more (e.g., two) polypeptide sequence having at least 90%, or at least 95% homology to SEQ ID NO:8 (g) one or more (e.g., two) polypeptide sequence having at least 90%, or at least 95% homology to SEQ ID NO: 11 and one or more (e.g., two) polypeptide sequence having at least 90%, or at least 95% homology to SEQ ID NO: 12; (h) one or more (e.g., two) polypeptide sequence having at least 90%, or at least 95% homology to SEQ ID NO: 13 and one or more (e.g., two) polypeptide sequence having at least 90%, or at least 95% homology to SEQ ID NO:14; (i) one or more (e.g., two) polypeptide sequence having at least 90%, or at least 95% homology to SEQ ID NO: 15 and one or more (e.g., two) polypeptide sequence having at least 90%, or at least 95% homology to SEQ ID NO: 16; or (j) one or more (e.g., two) polypeptide sequence having at least 90%, or at least 95% homology to SEQ ID NO: 17 and one or more (e.g., two) polypeptide sequence having at least 90%, or at least 95% homology to SEQ ID NO:18. In certain embodiments, the polypeptide comprises the CDRs present in any one of SEQ ID NOs: 1-18 or 19-25. In certain embodiments of the engineered WNT agonists, the one or more binding domains that bind to LRP5, LRP6, or both LRP5 and LRP6 are humanized. In certain embodiments, the engineered WNT agonists comprise a modified Fe domain, wherein the modified Fc domain comprises a LALAPG or N297G modification. In certain embodiments, the WNT agonist has any of the structures or formats disclosed herein, including any of the various antibody-related structures or formats. Examples of suitable formats include, but are not limited to, monoclonal antibodies (including full-length monoclonal antibodies), polyclonal antibodies, human antibodies, humanized antibodies, chimeric antibodies, nanobodies, diabodies, multi-specific antibodies (e.g., bispecific antibodies), and antibody fragments including but not limited to scFv, Fab, and Fab₂, so long as they exhibit the desired biological activity, e.g., WNT agonist activity. In particular embodiments, the WNT agonist is R2M13-h26. R2M13 is a humanized form of the parental R2M13-26 that also comprises the LALAPG substitution in the Fc domain. R2M13-h26 may also be referred to herein as R2M13-h26-LALAPG, R2M13-26 humanized LALAPG, or humanized LALPG.

In a related aspect, the disclosure provides a pharmaceutical composition comprising an engineered WNT agonist disclosed herein and a pharmaceutically acceptable carrier, diluent, or excipient.

In a further related aspect, the disclosure provides a method of treating a disease or disorder amenable to treatment by increased WNT pathway signaling in a subject, comprising administering to the subject an engineered WNT agonist or pharmaceutical composition disclosed herein. In particular embodiments, the disease or disorder is a gastrointestinal disorder, such as an inflammatory bowel disease. In certain embodiments, the disease or disorder is selected from the group consisting of: Crohn's disease (CD), CD with fistula formation, and ulcerative colitis (UC). In particular embodiments, the engineered WNT agonist is administered orally or parenterally, e.g., intravenously, intraperitoneally, or subcutaneously. In particular embodiments, the WNT agonist is R2M13-h26. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds.

In another related aspect, the disclosure provides a method of increasing WNT signaling in a cell, comprising contacting the cell with an engineered WNT agonist disclosed herein. In particular embodiments, the WNT agonist is R2M13-h26.

In another related aspect, the disclosure provides a method of modulating expression of a WNT pathway molecule in one or more tissues and/or cells in a subject having a gastrointestinal disorder, comprising administering to the subject an engineered WNT agonist or the pharmaceutical composition disclosed herein. In certain embodiments, the WNT pathway molecule is a gene or protein listed in any one of Tables 4-7. In particular embodiments, the WNT pathway molecule is selected from the group consisting of: RNAse4, Angiogenin, Gsta3, Rnf43, Axin2, or any of the genes or proteins listed in Table 7. In certain embodiments, expression of the WNT pathway molecule (gene or protein) is increased by at least 20%, at least 50%, at least 80%, at least 1.1-fold, at least 1.2-fold, at least 1.3-fold, at least 1.4-fold, at least 1.5-fold, two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In certain embodiments, the tissue is epithelial tissue. In certain embodiments, the cells are gastrointestinal epithelial cells, optionally: stem cells, TA1, TA2, basal goblet cells, injury-induced alternative progenitors (AltEnteroPC), injury-induced alternative enterocytes (AltEntero), enterocyte precursors (EnteroPrecur), goblet cells 1, goblet cells 2, or enteroendocrine or tuft cells. In particular embodiments, the WNT agonist is R2M13-h26. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds.

In another related aspect, the disclosure provides a method of stimulating tissue repair in a subject having a gastrointestinal disorder, comprising administering to the subject an engineered WNT agonist or the pharmaceutical composition disclosed herein. In particular embodiments, the tissue repair is stimulated by (or the method results in) modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4-8. In certain embodiments, the genes associated with the cell cycle are selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2. In certain embodiments, the genes associated with stem and progenitor cell renewal and differentiation are selected from those provided in Table 8, and Axin2, Id1, Hmga2, Nhp2, Foxq1, and Adh1. In certain embodiments, the genes associated with epithelial cell repair and barrier restoration are selected from those provided in Table 6, or Apex1, Agr2, B3gnt7, Fcgbp, Muc2, Muc3, Tff3, Zgl6, and Sprr2a3. In particular embodiments, expression of the gene is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds.

In another related aspect, the disclosure provides a method of reducing inflammation in a subject having a gastrointestinal disorder (or a tissue or cells thereof), comprising administering to the subject an engineered WNT agonist or the pharmaceutical composition disclosed herein. In certain embodiments, the inflammation is reduced by (or the method results in) modulation of at least one WNT pathway molecule selected from the group consisting of: genes provided in Table 5, or Adamdec1, Atf3, Gpx2, Gsta3, Gstm1, Gstm3, Gdf15, Ihh, Il18, Lyz2, Nox1, Reg4, Sycn, Selenbp1, Tgfbr2, and Timp3. In particular embodiments, the inflammation is reduced in gastrointestinal tissue, optionally epithelial tissue. In certain embodiments, the inflammation is reduced in gastrointestinal epithelial cells, epithelial stem cells, TA1, TA2, basal goblet cells, injury-induced alternative progenitors (Alt progenitors), injury-induced alternative enterocytes (Alt Enterocytes), enterocyte precursors (EnteroPrecur), goblet cells 1, goblet cells 2, or enteroendocrine or tuft cells. In particular embodiments, expression of the WNT pathway molecule is increased by at least 20%, at least 50%, at least 80%, at least 1.1-fold, at least 1.2-fold, at least 1.3-fold, at least 1.4-fold, at least 1.5-fold, two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds.

In particular embodiments of any of the methods disclosed, the engineered Wnt agonist is R2M13-h26 or comprises a functional variant or fragment thereof. In particular embodiments of any of the methods disclosed, the subject is a mammal, optionally a human.

In another related aspect, the disclosure provides a method of restoring gastrointestinal epithelial barrier in a subjecting having injured epithelium, comprising administering to the subject an engineered WNT agonist or pharmaceutical composition disclosed herein. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds. In some embodiments, the gastrointestinal epithelial barrier is restored by modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4, 5, 6, 7, 8, and 11. The genes associated with the cell cycle may be selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2. The genes associated with stem and progenitor cell renewal and differentiation may be selected from those provided in Table 8, and Axin2, Id1, Hmga2, Nhp2, Foxq1, and Adh1. The genes associated with epithelial cell repair and barrier restoration may be selected from those provided in Table 6, or Apex1, Agr2, B3gnt7, Fcgbp, Muc2, Muc3, Tff3, Zg16, and Sprr2a3.

In some embodiments, the gastrointestinal epithelial barrier is restored by modulation of at least one WNT pathway molecule, wherein expression of the WNT pathway molecule is increased by at least 20%, at least 50%, at least 80%, at least 1.1-fold, at least 1.2-fold, at least 1.3-fold, at least 1.4-fold, at least 1.5-fold, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In some embodiments, the gastrointestinal epithelial barrier is restored by modulation of at least one WNT pathway molecule, wherein expression of the WNT pathway molecule is increased in one or more tissues and/or cells of the subject within about 24 hours of administering the engineered Wnt agonist. In some embodiments, the subject's injured epithelium is substantially restored within about 6 days of administering the engineered Wnt agonist. In some embodiments, administration of the engineered Wnt agonist to the subject does not induce over proliferation of normal epithelium.

In another related aspect, the disclosure provides a method of inducing epithelial progenitor cell differentiation in a subject having a gastrointestinal disorder, comprising administering to the subject the engineered WNT agonist an engineered WNT agonist or the pharmaceutical composition disclosed herein. In certain embodiments, the WNT agonist is administered intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds. In some embodiments, the epithelial cell differentiation is induced by modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4, 5, 6, 7, 8, and 11. The genes associated with the cell cycle may be selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2. The genes associated with stem and progenitor cell renewal and differentiation may be selected from those provided in Table 8, and Axin2, Id1, Hmga2, Nhp2, Foxq1, and Adh1. The genes associated with epithelial cell repair and barrier restoration may be selected from those provided in Table 6, or Apex1, Agr2, B3gnt7, Fcgbp, Muc2, Muc3, Tff3, Zg16, and Sprr2a3.

In some embodiments, the epithelial cell differentiation is induced by modulation of at least one WNT pathway molecule, wherein expression of the WNT pathway molecule is increased by at least 20%, at least 50%, at least 80%, at least 1.1-fold, at least 1.2-fold, at least 1.3-fold, at least 1.4-fold, at least 1.5-fold, two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In some embodiments, the epithelial cell differentiation is induced by modulation of at least one WNT pathway molecule, wherein expression of the WNT pathway molecule is increased in one or more tissues and/or cells of the subject within about 24 hours of administering the engineered Wnt agonist.

In some embodiments, administration of the engineered Wnt agonist induces progenitor cell differentiation into enterocytes, goblet cells, enteroendocrine, or tuft cells in the subject. In some embodiments, substantial progenitor cell differentiation is induced in the subject within about 48 hours of administering the engineered Wnt agonist. In some embodiments, administration of the engineered Wnt agonist to the subject does not induce over proliferation of normal epithelium.

### BRIEF DESCRIPTION OF THE DRAWINGS

FIG. 1 provides an illustrative structure of one embodiment of an engineered WNT agonist. The R2M13 anti-Fzd5,8 antibody includes two heavy chains and two light chains, and each light chain also includes an anti-LRP6 VHH fused to its N-terminus via a tag.

FIG. 2A provides an amino acid sequence alignment of the parental LRP6 binding VHH, VHH26, and the closest human germline genes. CDR H1, H2, and H3 loop residues as defined by Kabat scheme are identified by bold lines above. Sequence alignment was performed using Clustal-Omega (https://www.ebi.ac.uk/Tools/msa/clustalo/). FIG. 2B provides an amino acid sequence alignment of the parental VHH26 and six different humanized variants thereof. CDR H1, H2, and H3 loop residues as defined by Kabat scheme are identified by bold lines above. Sequence alignment was performed using Clustal-Omega (https://www.ebi.ac.uk/Tools/msa/clustalo/).

FIGS. 3A-3B show biophysical characterization of the six humanized VHH26 variants (H1-H6). FIG. 3A shows SDS-PAGE of Ni-pull-down elution fractions from the metal-affinity chromatography. SEC and Octect-BLI profiles of VHH26-H1, VHH26-H2, VHH26-H3, VHH26-H4, VHH26-H5, and VHH26-H6 humanized variants are summarized in the table of FIG. 3B. Monomer % is based on SEC profile of the humanized VHH26 post ProA purification. ND=not determined.

FIG. 4 shows EC50 of binding to LRP5 or LRP6 of the indicated parental and variant VHH domains, in the context of the full engineered Wnt agonist format.

FIGS. 5A-5D show in vitro activity of Fzd5,8 subfamily specific Wnt mimetic R2M13-26:

FIG. 5A is a graph showing the binding affinity of the Fzd5,8 binder IgG of R2M13-26 to its target Fzd5 CRD measured on Octet.

FIG. 5B is a graph showing the binding affinity of the Fzd5,8 binder IgG of R2M13-26 to its target Fzd8 CRD measured on Octet.

FIG. 5C is a graph showing the binding specificity of the Fzd5,8 binder IgG of R2M13-26 to each of the 10 Fzd CRDs examined on Octet.

FIG. 5D is a graph showing the dose-dependent STF activities of R2M13-26, of the Fzd1,2,7-specific mimetic 1RC07-26, and of the Fzd1,2,5,7,8 pan specific mimetic R2M3-26, in the presence of 20 nM RSPO2 measured in Huh-7 cells.

FIG. 6 provides the sequences of the heavy chain and light chain present in the engineered WNT agonist, R2M13-h26. The heavy chain VH and light chain VL domains are underlined; the VHH26 domain is in italics; and CDR residues are in bold.

FIG. 7 provides a schematic diagram of a DSS model of acute colitis and resulting serum antibody exposure following treatment with various non-humanized and humanized versions, including: R2M13-03-LALAPG (non-humanized), R2M13-26-LALAPG (non-humanized), R2M13-36-LALAPG (non-humanized), R2M13-humanized-03-LALAPG, R2M13-humanized-26-LALAPG, R2M13-humanized-36-LALAPG, R2M13-humanized-03-N297G, and R2M13-humanized-36-N297G.

FIG. 8 provides graphs showing disease activity index of animals treated with the various non-humanized and humanized versions, including: R2M13-03-LALAPG (non-humanized), R2M13-26-LALAPG (non-humanized), R2M13-36-LALAPG (non-humanized), R2M13-humanized-03-LALAPG, R2M13-humanized-26-LALAPG, R2M13-humanized-36-LALAPG, R2M13-humanized-03-N297G, and R2M13-humanized-36-N297G. At time 10 days, the lines of the graph from top to bottom correspond to: R2M13-h03-LALAPG, anti-GFP, R2M13-h03-N297G, R2M13-03-LALAPG, R2M13-36-LALAPG, R2M13-h36-N297G (behind R2M13-h36-LALAPG), R2M13-h36-LALAPG, R2M13-h26-LALAPG, and no DSS, where “h” indicates humanized.

FIG. 9 provides graphs showing levels of cytokines in animals treated with the various controls and non-humanized and humanized versions, including from left to right: no DSS, anti-GFP, parental R2M13-03-LALAPG (non-humanized), parental R2M13-26-LALAPG (non-humanized), parental R2M13-36-LALAPG (non-humanized), R2M13-humanized-03-LALAPG, R2M13-humanized-26-LALAPG, R2M13-humanized-36-LALAPG, R2M13-humanized-03-N297G, and R2M13-humanized-36-N297G.

FIG. 10 provides a graph showing levels of lipocalin 2 of animals treated with the various controls and non-humanized and humanized versions, including: no DSS, anti-GFP, parental R2M13-03-LALAPG (non-humanized), parental R2M13-26-LALAPG (non-humanized), parental R2M13-36-LALAPG (non-humanized), R2M13-humanized-03-LALAPG, R2M13-humanized-26-LALAPG, R2M13-humanized-36-LALAPG, R2M13-humanized-03-N297G, and R2M13-humanized-36-N297G.

FIG. 11 provides micrographs showing restoration of epithelial tight junction marker, ZO-1, in vivo, in the DSS model of acute colitis, following treatment with the engineered WNT agonist. The brightly stained areas are ZO-1.

FIG. 12 provides micrographs showing repair of damaged colon epithelium in vivo, in the DSS model of acute colitis, following treatment with the engineered WNT agonist, R2M13-h26-LALPG as compared to control anti-GFP.

FIG. 13 provides micrographs showing restoration of the epithelial cell lineage including colonocytes, goblet cells, and tuft cells, in vivo, in the DSS model of acute colitis, following treatment with the engineered WNT agonist, R2M13-h26-LALPG as compared to control anti-GFP.

FIG. 14 provides a graph and table showing pharmacokinetics (PK) of the parental R2M13-26-LALAPG and humanized R2M13-26-LALAPG following intravenous injection as determined by measuring the amount of antibody in serum at various times following administration to rats, and compared to data obtained from mice.

FIG. 15 provides a schematic diagram of an acute chronic colitis DSS animal model system.

FIG. 16 provides graphs showing the disease activity index (DAI) of animals treated with R2M13-h26-LALAPG (R2M13-h26) or R2M13-26-LALAPG (R2M13-26). At time of 10 days, the lines of the graph from top to bottom correspond to: anti-GFP, cyclosporine A, R2M13-h26 (2 mpkx1), R2M13-h26 (20 mpkx1), R2M13-h26 (1 mpkx2), R2M13-h26 (6 mpkx1), R2M13-26 (3 mpkx2), R2M13-h26 (10 mpkx2), R2M13-26 (10 mpkx2), and no DSS.

FIG. 17 shows a cross section of transverse colon with H&E staining of animals treated R2M13-h26, as compared to anti-GFP or cyclosporin A.

FIG. 18 provides a diagram of a chronic DSS colitis animal model.

FIG. 19 shows micrographs of transverse colon section following the indicated treatment.

FIG. 20 provides graphs showing histology score and overall disease index following the indicated treatments.

FIG. 21 provides graphs showing lipocalin-2 and IL-6 expression following the indicated treatments.

FIG. 22 is a diagram of a chronic DSS colitis animal model.

FIG. 23 provides graphs showing disease activity index of animals treated with R2M13-h26 or IL12/23p40.

FIG. 24 provides graphs showing expression of the indicated cytokines in animals treated with R2M13-h26 or IL12/23p40.

FIG. 25 provides graphs showing Axin2 and Ki67 expression following the indicated treatments with R2M13-26-LALAPG (R2M13-26).

FIGS. 26A-26C shows the different cell types detected in the colon from scRNA-seq on uninjured and DSS-treated mice:

FIG. 26A is a schematic diagram showing the experimental design of the scRNA seq experiment.

FIG. 26B is a plot of the first two principal components: the lineage/tissue layer is indicated, showing the three groups radiating from the center.

FIG. 26C provides graphs showing the strong impact the DSS injury had on number of differential genes expressed in different tissue layer/lineages. The graph on the left shows the number of differentially expressed genes from each tissue layer on Day 5 and Day 6 of DDS mice compared to uninjured mice; the graph on the right shows the number of differentially expressed genes from each tissue layer on Day 5 and Day 6 of treatment with R2M13-26 compared to Anti-GFP. The tissues/lineages from top to bottom of each bar correspond to epithelium, immune, and stroma, with almost all epithelium following treatment with R2M13-26-LALAPG (R2M13-26) at day 5.

FIGS. 27A-27C shows that while DSS impacts all tissue layers by day 5, the predominant effect of R2M13-26-LALAPG (R2M13-26) is on the epithelium at 24-hours after treatment on day 5. FIGS. 27A-27C show R2M13-26-LALAPG (R2M13-26) increased Wnt target and cell cycle gene expression and expanded the progenitors in the epithelium after injury.

FIG. 27A is a table listing selected top gene sets (from GSEA) enriched in the R2M13-26 treated DSS-injured epithelium relative to the anti-GFP treated DSS-injured epithelium.

FIGS. 27B and 27C show validation of the scRNA-seq analysis in the tissue.

FIG. 27B shows RNA in situ hybridization of two Wnt target genes, Axin2 and Cdkn3, in the uninjured, DSS/anti-GFP and DSS/R2M13-26 treatment groups (day 5); nuclei labeled with DAPI. Scale bar represents 100 microns.

FIG. 27C shows immunohistochemistry for the proliferative cell marker, MKI67, in the uninjured, DSS/anti-GFP, and DSS/R2M13-26 treated colon samples (day 6); nuclei labeled with DAPI. Scale bar represents 100 microns.

FIGS. 28A-28E show R2M13-26-LALAPG (R2M13-216) treatment caused accelerated, proper differentiation in the DSS model:

FIGS. 28A-D provide graphs showing uniform manifold approximation projection (UMAP) plots of the epithelial cells.

FIG. 28A is a graph showing UMAP of epithelial cells colored by cluster/cell type.

FIG. 28B is a graph showing UMAP colored by experimental condition of the cells.

FIG. 28C is a graph showing a minimum spanning tree of the cluster medoids, connecting clusters based on similarity. Only the cell types that were not populated almost exclusively by injured cells were included. The stem cell and TA2 cell types were merged and set as the starting cluster.

FIG. 28D is a graph showing the completed slingshot-predicted lineage trajectory indicating a transition from the stem cell/TA cells to the EnteroPrecur cells on the way to the immature and mature enterocytes (going up); and bifurcating from the stem cell/TA cells down to go either toward tufted cells or toward goblet and enteroendocrine cells with a second bifurcation between them from the goblet progenitor cell type.

FIG. 28E provides histograms of the number of cells from the indicated treatment groups at the 48-hour/day 6 timepoint at the indicated position along the pseudotime or lineage trajectory axis derived from the enterocyte lineage presented in FIG. 28D. The vertical red dashed line represents the same position along the axis in all three plots while the distribution shows how many cells are present at the position. The pseudotime order (x-axis) is the same in each plot and is ordered from left to right. FIG. 28E shows that the progression toward enterocyte lineage is increased with R2M13-26-LALPG (R2M13-26) treatment.

FIGS. 29A-29L and 29A′-29L′ show that the Frizzled family of receptors presented differential expression patterns in the small intestinal epithelium:

FIGS. 29A-29L provide graphs showing expression of each of the 10 Fzd receptors (Fzd1-10), Axin2, and Lgr5, respectively, in the normal duodenum as determined by RNAscope in situ hybridization.

FIGS. 29A′-29′L provide graphs with zoomed in views showing Fzd expression in the small intestinal crypts. Arrows in panel E′ indicate intestinal stem cells.

FIGS. 30A-30T show that the Frizzled family of receptors were expressed at different levels in the colon:

FIGS. 30A-30J provide graphs showing colon expression of the 10 Fzd receptors in naïve mice examined by RNAscope in situ hybridization.

FIGS. 30K-30T provide graphs showing colon expression of the 10 Fzd receptors in mice treated with 7 days of 4% DSS.

FIG. 31 shows a reduction of inflammation by a reduction of the neutrophil infiltrate. S100A9 is a marker of neutrophil infiltration, and CD45 is a marker of activated inflammatory cells.

FIG. 32 provides a graph showing increased serum ALP following administration of the indicated dosages of R2M13-h26.

FIG. 33 is a schematic drawing depicting a pharmacokinetic assay used to measure mean serum concentration of R2M13-h26.

FIG. 34 provides a graph showing mean serum concentrations of R2M13-h26 in groups 2-4.

FIG. 35 provides a graph showing individual serum R2M13-h26 concentrations measured following the first dose. The arrow points to two animals in the 30 mg/kg dose group with accelerated clearance starting 3 days after dosing.

FIGS. 36A-36B provide a pair of graphs showing ALP increase in days 0-7 (FIG. 36A) and days 28-42 (FIG. 36B) for different dosage groups of R2M13-h26.

FIG. 37 provides a graph showing mean serum R2M13-h26 concentrations after a single dose of R2M13-h26.

FIG. 38 provides a table showing PK parameters for R2M13-h26 after a single dose of R2M13-h26.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. Definitions

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, to tissues, to tissue organoids, and/or to organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, VHH antibodies, camelid antibodies, nanobodies, diabodies, multi-specific antibodies (e.g., bispecific antibodies), and antibody fragments including but not limited to scFv, Fab, and Fab2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and 30 additionally capable of being used in an animal to produce antibodies capable of binding to an epitope of that antigen. In certain embodiments, a binding agent (e.g., a Engineered WNT agonist or binding region thereof, or a WNT antagonist) is said to specifically bind an antigen when it preferentially recognizes its target antigen in a complex mixture of proteins and/or macromolecules.

The term “antigen-binding fragment” as used herein refers to a polypeptide fragment that contains at least one CDR of an immunoglobulin heavy and/or light chain, or of a Nanobody® (Nab), that binds to the antigen of interest, in particular to one or more FZD receptors, or to LRP5 and/or LRP6. In this regard, an antigen-binding fragment of the herein described antibodies may comprise 1, 2, 3, 4, 5, or all 6 CDRs of a VH and VL from antibodies that bind one or more FZD receptors or LRP5 and/or LRP6.

As used herein, the terms “biological activity” and “biologically active” refer to the activity attributed to a particular biological element in a cell. For example, the “biological activity” of an WNT agonist, or fragment or variant thereof refers to the ability to mimic or enhance WNT signals. As another example, the biological activity of a polypeptide or functional fragment or variant thereof refers to the ability of the polypeptide or functional fragment or variant thereof to carry out its native functions of, e.g., binding, enzymatic activity, etc. In some embodiments, a functional fragment or variant retains at least 20%, at least 30%, at least 40%, at least 50%, at least 60%, at least 70%, at least 80%, at least 90%, or at least 100% of an activity of the corresponding native protein or nucleic acid. As a third example, the biological activity of a gene regulatory element, e.g. promoter, enhancer, Kozak sequence, and the like, refers to the ability of the regulatory element or functional fragment or variant thereof to regulate, i.e. promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “epitope” is specific region on an antigen that an antibody recognizes and binds to, and is also referred to as the “antigenic determinant”. An epitope is usually 5-8 amino acids long on the surface of the protein. Proteins are three dimensionally folded structures, and an epitope may only be recognized in its form as it exists in solution, or its native form. When an epitope is made up of amino acids that are brought together by the three-dimensional structure, the epitope is conformational, or discontinuous. If the epitope exists on a single polypeptide chain, it is a continuous, or linear epitope. Depending on the epitope an antibody recognizes, it may bind only fragments or denatured segments of a protein, or it may also be able to bind the native protein.

The portion of an antibody or antibody fragment thereof that recognizes an epitope is referred to as the “epitope binding domain” or “antigen binding domain”. The epitope or antigen binding domain of an antibody or antibody fragment is in the Fab fragment and the effector functions in the Fc fragment. Six segments, known as complementarity determining regions (CDRs) within the variable regions (VH and VL) of the heavy and light chains loop out from the framework (FR regions) globular structure of the rest of the antibody and interact to form an exposed surface at one end of the molecule. This is the antigen binding domain. Generally, 4-6 of the CDRs will be directly involved in binding antigen, although fewer can provide the main binding motifs.

An “expression vector” is a vector, e.g. plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g., promoters, enhancers, UTRs, miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

“Humanized” antibodies or fragments thereof refers to antibodies or fragments thereof from non-human species whose protein sequences have been modified to increase their similarity to antibody variants produced naturally in humans. The process of “humanization” is usually applied to monoclonal antibodies developed for administration to humans.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Engineered WNT agonists disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described herein or under the definition of “antibody”.

The term “native” or “wild-type” as used herein refers to a nucleotide sequence, e.g. gene, or gene product, e.g. RNA or protein, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full-length native polynucleotide sequence. As another example, a variant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. As used herein, the terms “identity” and “identical” refer, with respect to a polypeptide or polynucleotide sequence-of-interest, to the percentage of exact matching residues in an alignment of that the sequence-of-interest to a reference sequence, such as an alignment generated by the BLAST algorithm. Identity is calculated, unless specified otherwise, across the full length of the reference sequence. Thus a sequence-of-interest “shares at least x % identity to” a reference sequence if, when the reference sequence is aligned (as a query sequence) is aligned to the sequence-of-interest (as subject sequence), at least x % (rounded down) of the residues in the subject sequence are aligned as an exact match to a corresponding residue in the query sequence, the denominator being the full length of the reference sequence plus the lengths of any gaps inserted into the reference sequence by alignment of the reference sequence to the sequence-of-interest. Where the subject sequence has variable positions (e.g., residues denoted X), an alignment to any residue in the query sequence is counted as a match.

Sequence similarity can be determined in a number of different manners. To determine sequence identity, sequences can be aligned using the methods and computer programs, including BLAST, available over the worldwide web at ncbi.nlm.nih.gov/BLAST/. Sequence alignments may be performed using the NCBI Blast service (BLAST+ version 2.12.0) or another program giving the same results. Unless indicated to the contrary, sequence identity is determined using the BLAST algorithm (e.g., bl2seq) with default parameters.

Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### II. General

The present invention provides compositions and methods of modulating WNT signals to ameliorate various diseases and disorders that may benefit from modulation of WNT signaling pathways, such as gastrointestinal disorders, including but not limited to, inflammatory bowel disease, including but not limited to, Crohn's disease, Crohn's disease with fistula formation, and ulcerative colitis.

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of WNT signaling pathways has potential for treatment of degenerative diseases and tissue injuries.

One of the challenges for modulating WNT signaling as a therapeutic is the existence of multiple WNT ligands and WNT receptors, Frizzled 1-10 (FZD1-10), with many tissues expressing multiple and overlapping FZDs. Canonical WNT signaling also involves Low-density lipoprotein (LDL) receptor-related protein 5 (LRP5) and/or Low-density lipoprotein (LDL) receptor-related protein 6 (LRP6) as co-receptors, which are broadly expressed in various tissues, in addition to FZDs. LRP5 and LRP6 are collectively referred to as LRP5/6, and reference to “LRP5/6 binding,” or the like, indicates binding to LRP5 and/or LRP6.

R-spondins 1-4 (RSPO1-4) are a family of ligands that amplify WNT signals. Each of the R-spondins works through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight and Hankenson 2014, Matrix Biology; 37: 157-161). R-spondins might also work through additional mechanisms of action. ZNRF3 and RNF43 are two membrane-bound E3 ligases specifically targeting WNT receptors (FZD1-10 and LRP5 or LRP6) for degradation. Binding of an R-spondin to ZNRF3/RNF43 and LGR4-6 causes clearance or sequestration of the ternary complex, which removes E3 ligases from WNT receptors and stabilizes WNT receptors, resulting in enhanced WNT signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying WNT signaling. While R-spondin effects depend on WNT signals, since both LGR4-6 and ZNRF3/RNF43 are widely expressed in various tissues, the effects of R-spondins are not tissue-specific.

Activating WNT signaling by a WNT agonist may be used for the treatment of a variety of diseases and disorders, including gastrointestinal disorders. Similarly, amplifying WNT signaling by RSPO or an RSPO mimetic may be used for the treatment of a variety of diseases and disorders, including gastrointestinal disorders. Previous work in the literature suggests RSPO may be used for the treatment of experimental colon colitis (J. Zhao et. al., 2007). A WNT agonist molecule may also be used for the treatment of gastrointestinal disorders. In particular, active WNT signaling can provide a major stem cell maintenance signal and plays a key role in regulating regeneration of the intestinal epithelium in homeostasis and in injury.

The two intestinal epithelial lineages, absorptive and secretory, define the two main functions of the gut apparatus. Secretory cells secrete hormones and provide an important barrier against food-borne microorganisms, toxins, and antigens, mainly through the secretion of mucus and anti-microbial peptides. In contrast, the absorptive cells conduct uptake of dietary nutrients, as they localize mainly at the tips of the villi in the small intestine or at the top of the colonic crypts, thus constituting the majority of luminal cells across the intestinal surface area (see, e.g., Santos, et. al (2018) Trends in Cell Biol. in press, https://doi.org/10.1016/j.tcb.2018.08.001). Under homeostasis conditions, all cells in the intestinal epithelium regenerate in 3-10 days.

Different niche factors maintain intestinal stem cell (ISC) activity, and distinct non-epithelial and/or epithelial cells elaborate various signals that make up a cellular niche. Such niche factors include not only canonical signals such as WNT, R-spondin, Notch, and Bone Morpohogenetic Protein (BMP), but also inflammatory and dietary influences. Upon injury, the ISC niche adapts beyond its homeostatic state to interpret pathogenic stimuli and translate them into regeneration of the epithelium. This regeneration is mediated by either surviving Lgr5+ ISCs or other mature cell types such as enterocytes, enteroendocrine, or Paneth cells that can convert back to Lgr5+ ISCs to aid epithelial regeneration (Beumer and Clevers (2016), Development 143: 3639-3649).

ISCs at the bottom of the intestinal crypt, also known as columnar base cells (CBCs), are intercalated with WNT secreting Paneth cells (Cheng and Leblond (1974) Am. J. Anat. 141: 537-561). Mesenchymal cells surrounding the intestinal epithelium also secrete some WNT proteins, serving an overlapping stem cell niche function in vivo (Farin, el. al (2012) Gastroenterol. 143: 1518-1529). In the presence of WNT signaling, ISCs divide to produce self-renewing stem cells and differentiating daughter cells, which first go through a few fast transit amplifying (TA) divisions before differentiating into functional cell types. There is also a quiescent stem cell population in the intestinal crypt, +4 cells, which can contribute to epithelial regeneration when CBCs are damaged (Tian, el. al (2011) Nature 478: 255-259). Commitment to individual lineage and terminal differentiation take place as the TA cells migrate out along the crypt-villus axis, away from the WNT producing cells.

### III. Engineered WNT Agonists

The present disclosure provides engineered WNT agonists and contemplates the use of engineered WNT agonists to stimulate, agonize, or promote WNT signaling, e.g., through the canonical WNT/β-catenin signaling pathway. Such engineered WNT agonists may also be referred to as WNT/β-catenin signaling agonists or Wnt mimetics.

Several challenges exist in engineering Wnt proteins for clinical applications. First, Wnt proteins are difficult to produce and do not contain typical drug-like properties. Second, it was reported that in vivo overexpression or application of exogenous RSPO, which amplifies Wnt signaling, helped regenerate intestine epithelium in various injury models (Zhao et al., 2007), but it was also reported to induce increased proliferation of normal intestine epithelium (Yan Kelley S. et al., 2017).

The disclosure addresses the first challenge by providing synthetic Wnt mimetics with drug-like properties, particularly in the form of recombinant, bi-specific antibodies that bring together Fzd and Lrp to stimulate signaling, mimicking endogenous Wnt ligands. The Wnt mimetics of the disclosure may freely diffuse, access damaged tissues and guide tissue repair where Wnt signals are needed.

The disclosure addresses the second challenge by providing Wnt mimetics that are capable of repairing damaged intestine epithelium without being combined with RSPO. Unlike RSPO, the Wnt mimetics of the disclosure do not induce hyperproliferation of normal intestine epithelium.

Wnt mimetics of the disclosure have the desired properties of restoring diseased intestine tissue back to normal physiology. In some embodiments, Wnt mimetics of the disclosure induce rapid restoration of damaged epithelial tissue. In some embodiments, damaged epithelial barrier may be restored within about 10 days, about 8 days, about 7 days, about 6 days, or about 5 days of treatment with Wnt mimetics of the disclosure. In some embodiments, damaged epithelial barrier may be restored within about 6 days of treatment with Wnt mimetics of the disclosure. In some embodiments, Wnt mimetics of the disclosure induce expression of Wnt target genes in injured epithelial cells within about 12 hours, about 24 hours, about 36 hours, or about 48 hours. In some embodiments, Wnt mimetics of the disclosure induce expression of Wnt target genes in injured epithelial cells within about 24 hours. In some embodiments, the Wnt target genes that are induced by Wnt mimetics of the disclosure comprise Axin2, Rnf43, Cdkn3. In some embodiments, expression of Axin2 in injured epithelial cells is induced by Wnt mimetics of the disclosure within about 24 hours.

In some embodiments, the Wnt agonists disclosed herein support the proliferation and differentiation of stem cells in the damaged intestinal or colonic crypts of patients with moderate to severe IBD. In some embodiments, the Wnt agonists disclosed herein have the potential to accelerate the repair of the intestinal barrier, which can result in a reduction of bacteria penetrating through the intestinal epithelium and a reduction of immune cell activation and inflammation, thereby treating inflammatory bowel diseases.

In some embodiments, the Wnt agonists disclosed herein have several simultaneous beneficial effects: activate the Wnt signaling pathway in intestinal stem cells and progenitor cells resulting in proliferation and differentiation; restore intestinal barrier function and tissue architecture; reduce tissue inflammation; and reduce disease activity in moderate to severe IBD.

In some embodiments, the Wnt agonists disclosed herein is a bispecific antibody targeting Fzd5/8 and Lrp6. Fzd5 was previously reported to be highly expressed in intestinal mucosal cells from IBD patients. Fzd5 was also highly expressed in a mouse model of colitis induced by dextran sodium sulfate (DSS). In some embodiments, the Wnt agonists disclosed herein binds to DSS-injured intestinal cells, stimulating Wnt signaling as measured by the expression of Axin2, a downstream target gene in the Wnt pathway. In some embodiments, the Wnt agonists disclosed herein binds to Fzd5/8 and Lrp6 on intestinal stem cells to activate Wnt signaling.

In some embodiments, administration of a Wnt agonist disclosed herein improves in the disease activity index, or DAI, in a DSS model. The DAI is a composite score composed of body weight change, diarrhea, and bloody stools that is frequently used to quantify disease severity in preclinical rodent models and in the clinic. In some embodiments, administration of a Wnt agonist disclosed herein leads to a dose dependent decrease in DAI. In some embodiments, treatment with a Wnt agonist disclosed herein is superior to treatment with cyclosporine, an anti-TNF antibody, or an anti-IL12/23 antibody. In some embodiments, administration of a Wnt agonist disclosed herein improves the DAI in both a chronic DSS model and an acute DSS model.

In some embodiments, Wnt mimetics of the disclosure expand progenitor cell populations in the epithelium. In some embodiments, Wnt mimetics of the disclosure expand progenitor cell populations by increasing the expression of cell cycle genes in said cell populations. The progenitor cell populations may include, for example, normal progenitors responding to injury and progenitors in altered cell states, such as de-differentiation. In some embodiments, Wnt mimetics of the disclosure substantially expand progenitor cell populations in the epithelium within about 24 hours.

In some embodiments, Wnt mimetics of the disclosure accelerates differentiation of progenitor cells into mature cell types. In some embodiments, Wnt mimetics of the disclosure accelerates differentiation of progenitor cells, e.g., gastrointestinal progenitor cells, to enterocytes, goblet cells, enteroendocrine, or tuft cells. In some embodiments, Wnt mimetics of the disclosure accelerate differentiation of progenitor cells to enterocytes. In some embodiments, substantial differentiation of progenitor cells into mature cell types occurs within about 24 hours, about 36 hours, about 48 hours, or about 60 hours of treatment with Wnt mimetics of the disclosure. In some embodiments, substantial differentiation of progenitor cells into mature cell types occurs within about 48 hours of treatment with Wnt mimetics of the disclosure. In some embodiments, Wnt mimetics of the disclosure accelerate differentiation of progenitor cells into mature cell types while reducing expression of high levels of inflammatory genes.

In some embodiments, the breakdown of the intestinal barrier triggers influx of luminal pathogen and an inflammatory response that leads to further tissue damage. Disease modification in IBD can be measured by the levels of inflammatory cytokines present in the injured tissue and in serum. In some embodiments, treatment of epithelial tissue injury with Wnt mimetics of the disclosure reduces production of inflammatory cytokines. In some embodiments, treatment of damaged epithelial tissue with Wnt mimetics of the disclosure reduces inflammatory cytokine production by at least about 1%, at least about 2%, at least about 5%, at least about 10%, at least about 15%, at least about 20%, at least about 25%, at least about 30% at least about 35%, at least about 40%, at least about 45%, at least about 50%, at least about 60%, at least about 70%, at least about 80%, or at least about 90%, compared to a treatment that does not comprise Wnt mimetics of the disclosure, or compared to no treatment.

In some embodiments, the disclosure provides a Wnt mimetic capable of effectively repairing injured epithelium without inducing over proliferation of normal epithelium. In some embodiments, Wnt mimetics of the disclosure alone does not affect proliferation of normal epithelium. In some embodiments, the epithelium is colon or small intestine epithelium. In some embodiments, the disclosure provides a Wnt mimetic capable of repairing injured epithelium with higher efficacy than a treatment comprising RSPO. In some embodiments, the disclosure provides a Wnt mimetic capable of repairing injured epithelium with higher efficacy than a treatment comprising RSPO and a Wnt mimetic of the disclosure. In some embodiments, the Wnt mimetic of the disclosure improves injured epithelium with better efficacy than a treatment comprising RSPO, or a treatment comprising RSPO and a Wnt mimetic of the disclosure, by at least about 1%, at least about 2%, at least about 5%, at least about 10%, at least about 15%, at least about 20%, at least about 25%, at least about 30% at least about 35%, at least about 40%, at least about 45%, at least about 50%, at least about 60%, at least about 70%, at least about 80%, or at least about 900%. Efficacy of repairing injured epithelium may be determined by histology severity scores wherein a higher score indicates more severe damage, or by disease activity index (DIA), which may be calculated based on the average score of weight loss, stool consistency and the degree of intestinal bleeding.

In some embodiments, the disclosure provides a Fzd5,8 and Lrp6-specific Wnt mimetic—(for example, R2M13-26 or R2M13-h26). In some embodiments, the Fzd5,8 and Lrp6-specific Wnt mimetic of the disclosure is capable of activating Wnt signaling on epithelial cells. Activation of Wnt signaling may be measured by gene expression using scRNA-seq (single-cell RNA sequencing) methods known in the art and described in the disclosure. In some embodiments, the epithelium cells are colon or small intestine epithelium cells. In some embodiments, the epithelial cells include multiple stem or progenitor cells.

The engineered WNT agonists include one or more binding domain that binds to one or more FZD or an epitope thereof, and one or more binding domain that binds to one or more of LRP5 and/or LRP6, or an epitope within LRP5 and/or LRP6. In certain embodiments, the engineered WNT agonist specifically binds to the cysteine-rich domain (CRD) within the human frizzled receptor(s) to which it binds.

In certain embodiments, the engineered WNT agonists may comprise one or more additional binding domain. For example, they may comprise one or more binding domains that bind to one or more of the E3 ligases, ZNRF3/RNF43, or specific epitopes within either of the E3 ligases. In certain embodiments, the E3 ligase binding domain comprises an R-SPO or a fragment thereof.

In certain embodiments, the engineered WNT agonists may comprises one or more tissue-specific or cell type-specific binding domain that specifically binds to a target tissue or cell type.

In one aspect, the disclosure provides VHH domains that bind to LRP5 and/or LRP6. Illustrative sequences of these VHH domains are provided in Table 1. The VHH binding domains may be derived from any of the disclosed sequences. In particular embodiments, the VHH binding domains are humanized. The present disclosure contemplates engineered WNT agonists that comprise one of more disclosed VHH domain, including any of the humanized VHH domains disclosed herein, as well functional fragments and variants of such VHH domains having at least 80%, at least 90%, at least 95%, at least 98%, or at least 99% sequence identity to any of the VHH sequences disclosed herein. In certain embodiments, a VHH domain comprises three CDR sequences: GRIFAIYDIA, IRPVVTEIDYADSVKG, and RPWGSRDEY. In certain embodiments, an engineered Wnt agonist comprises a VHH domain having at least 80%, at least 85%, at least 90%, at least 95%, at least 98%, or at least 99% sequence identity to any one of SEQ ID NOs: 19-25. In particular embodiments, the engineered Wnt agonist is a bi-specific antibody-like molecule comprising an IgG structure comprising two heavy chains and two light chains, wherein VHH domains are fused to the N-terminus of each light chain present in the antibody-like molecule. In particular embodiments, the heavy chain is effector-less, e.g., contains LALAPG mutations.

In another aspect, the disclosure provides FZD binding domains that bind to one or more FZD. Illustrative sequences of these FZD binding domain are provided in Table 3, in the context of VH and VL domains derived from an anti-FZD antibody, R2M13. The present disclosure contemplates engineered WNT agonists that comprise one or more of the VH or VL domains disclosed herein, as well functional fragments and variants of such VH or VL domains having at least 80%, at least 90%, at least 95%, at least 98%, or at least 99% sequence identity to any of the VH or VL sequences disclosed herein. In addition, the present disclosure contemplates engineered WNT agonists that comprise one or more of the heavy or light chain sequences provided in Table 3, as well functional fragments and variants of such heavy or light chains having at least 80%, at least 90%, at least 95%, at least 98%, or at least 99% sequence identity to any of the heavy or light chain sequences disclosed herein. In certain embodiments, a FZD binding domain comprises three light chain CDR sequences: RASQSISSYLN (CDRL1), AASSLQS (CDRL2), and QQSYSTPLT (CDRL3), and/or three heavy chain light chain CDR sequences: GGTFTYRYLH (CDRH1), GIIPIFGTGNYAQKFQG (CDRH2), and SMVRVPYYYGMDV (CDRH3), any CDRs provided herein.

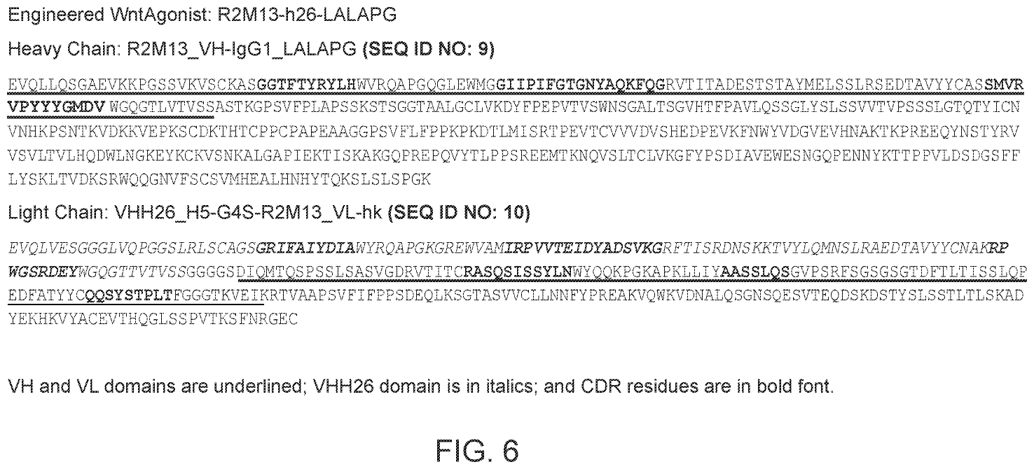

In related embodiments, the disclosure contemplates engineered WNT agonists comprising one or more CDRs present in a FZD binding domain or LRP5/6 binding domain disclosed herein: e.g., one or more (e.g., two or three) of the VHH CDRs shown in FIG. 6 or Table 1; one or more (e.g., two or three) of the CDRs present in a heavy chain or light chain disclosed herein. In certain embodiments, the engineered WNT agonists comprise 4, 5, or all six of the CDRs shown for a FZD binding domain disclosed herein, e.g., in FIG. 6 or Table 3. In certain embodiments, the engineered WNT agonists comprises 6, 7, 8, or all 9 of the CDRs shown for an engineered WNT agonists disclosed herein e.g., in FIG. 6 or Table 3.

The disclosure provides polypeptides comprising or consisting of a sequence having at least 80%, at least 85%, at least 90%, at least 95%, at least 98%, or at least 99% identity to a binding domain provided herein, as well as polypeptides comprising two or more, e.g., three, of the CDR sequences disclosed herein, such as a polypeptide comprising the following CDRs: GRIFAIYDIA, IRPVVTEIDYADSVKG, and RPWGSRDEY (VHH CDRs1-3, respectively), and which binds to LRP5 or LRP6, or a polypeptide comprising the following CDRs: RASQSISSYLN (CDRL1), AASSLQS (CDRL2), and QQSYSTPLT (CDRL3), which, in combination with a heavy chain, binds one or more FZD, or a polypeptide comprising the following CDRs: GGTFTYRYLH (CDRH1), GIIPIFGTGNYAQKFQG (CDRH2), and SMVRVPYYYGMDV (CDRH3), which, in combination with a light chain, binds one or more FZD. The disclosure also includes a FZD binding domain comprising two heavy chains and two light chains, wherein each heavy chain comprises two or more of the following CDRs: GGTFTYRYLH (CDRH1), GIIPIFGTGNYAQKFQG (CDRH2), and SMVRVPYYYGMDV (CDRH3), and each light chain comprises two or more of the following CDRs: RASQSISSYLN (CDRL1), AASSLQS (CDRL2), and QQSYSTPLT (CDRL3), wherein the FZD binding domain binds to one or more FZD. In certain embodiments, the FZD binding domain is an antibody, and the heavy chain further comprises an Fc domain, e.g., an IgG1 Fc domain, which may be modified. The disclosure further provides polypeptides comprising or consisting of a sequence having at least 80%, at least 85%, at least 90%, at least 95%, at least 98%, or at least 99% identity to a variable heavy or variable light domain disclosed herein e.g., in SEQ ID NOs: 1-25, FIG. 6 or Table 3. The disclosure further provides polypeptides comprising or consisting of a sequence having at least 80%, at least 85%, at least 90%, at least 95%, at least 98%, or at least 99% identity to a VHH domain disclosed herein e.g., in SEQ DI NOs: 1-25, FIG. 6 or Table 3. The disclosure further provides polypeptides comprising or consisting of a sequence having at least 80%, at least 85%, at least 90%, at least 95%, at least 98%, or at least 99% identity to a heavy chain or light chain or fusion polypeptide disclosed herein e.g., in SEQ ID NOs: 1-25, FIG. 6 or Table 3. In embodiments of any of the polypeptide variants disclosed herein, the CDRs are not modified as compared to the original or parental sequence.

In additional the disclosure provides polynucleotide sequences encoding any of the polypeptides described herein, as well as functional fragments and variants thereof, e.g., fragments and variants that bind one or more FZD or LRP5/6, VH domains, and VL domains.

In certain embodiments, an engineered WNT agonist disclosed herein comprises an Fc domain (e.g., as part of a heavy chain). In particular embodiments, the Fc domain is engineered to include specific amino acid substitutions, including those corresponding to LALAPG or N297G.

In particular embodiments of the engineered WNT agonists, one or more LRP5/6 binding domain disclosed herein (e.g., any of VHH26-H1-H6) is fused to one or more of the light chain or heavy chain of a FZD binding domain disclosed herein (e.g., an R2M13 derived FZD binding domain), e.g., directly or via a linker, e.g., a peptide linker. However, in other embodiments, any LRP5/6 binding domain disclosed herein may be fused to or complexed with a different FZD binding domain to achieve an engineered WNT agonist, and any FZD binding domain disclosed herein may be fused to or complexed with a different LRP5/6 binding domain to achieve an engineered WNT agonist. A variety of anti-FZD or anti-LRP antibodies that may be present in whole or in part in an engineered WNT agonist disclosed herein include those described in U.S. Pat. No. 7,462,697, PCT Publication No. WO 2019/126399, and PCT Publication No. WO 2019/126401. Illustrative formats and sequences are also provided in PCT Publication No. WO 2019/126398, each of which is incorporated herein in its entirety.

Engineered WNT agonists may adopt a variety of different structural conformations, each comprising one or more, e.g., two, FZD binding domains and one or more, e.g., two) LRP5/6 binding domains. The FZD binding domain(s) and LRP5/6 binding domain(s) may be directly fused to each other or via a linker, e.g., a peptide linker. Alternatively, the FZD binding domain(s) and LRP5/6 binding domain(s) may be complexed to each other.

In certain embodiments, the engineered WNT agonist comprises two heavy chains and two light chains, wherein the light chain comprises a fused VHH, and adopts an antibody-like confirmation, wherein the two heavy chains are bound to each other via disulfide bonds and the two light chains are bound to the heavy chains via disulfide bonds.

The engineered WNT agonists may adopt other antibody-like structures or confirmations, including those found in various functional fragments, including but not limited to any of those disclosed herein.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)2, Fv, single chain (scFv), camelid antibodies, Nanobodies® (Nabs; also known as sdAbs or VHH domains), DVD-Igs, synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including Engineered WNT agonists disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

In certain embodiments, single chain Fv or scFV antibodies are contemplated for use in the engineered Wnt agonists. For example, Kappa bodies (Ill et al., Prot. Eng. 10: 949-57 (1997)); minibodies (Martin et al., EMBO J 13: 5305-9 (1994)); diabodies (Holliger et al., PNAS 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptors through one binding domain and to a second molecule through a second binding domain. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated-light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., Nature 341, 544-546 (1989)).

Where bispecific antibodies are to be used, these may be conventional bispecific antibodies, which can be manufactured in a variety of ways (Holliger, P. and Winter G., Current Opinion Biotechnol. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, the antibodies of the present disclosure may take the form of a single domain (sdAb) or VHH antibody fragment (also known as a Nanobody®). The sdAb or VHH technology was originally developed following the discovery and identification that camelidae (e.g., camels and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of “Nanobodies®”. The sdAbs or VHHs are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula or Pichia (see, e.g., U.S. Pat. No. 6,838,254). The production process is scalable and multi-kilogram quantities of Nanobodies® have been produced. sdAbs or VHHs may be formulated as a ready-to-use solution having a long shelf life. The Nanoclone® method (see, e.g., WO 06/079372) is a proprietary method for generating Nanobodies® against a desired target, based on automated high-throughput selection of B-cells. sdAbs or VHHs are single-domain antigen-binding fragments of camelid-specific heavy-chain only antibodies.

Another antibody fragment contemplated is a dual-variable domain-immunoglobulin (DVD-Ig) is an engineered protein that combines the function and specificity of two monoclonal antibodies in one molecular entity. A DVD-Ig is designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. A DVD-Ig can be produced by conventional mammalian expression systems as a single species for manufacturing and purification. A DVD-Ig has the specificity of the parental antibodies, is stable in vivo, and exhibits IgG-like physicochemical and pharmacokinetic properties. DVD-Igs and methods for making them are described in Wu, C., et al., Nature Biotechnology, 25:1290-1297 (2007)).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327).

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

In some embodiments, Engineered WNT agonist comprises one or more Fab or antigen-binding fragment thereof and one or more VHH or sdAb or antigen-binding fragment thereof (or alternatively, one or more scFv or antigen-binding fragment thereof). In certain embodiments, the Fab specifically binds one or more Fzd receptor, and the VHH or sdAb (or scFv) specifically binds LRP5 and/or LRP6. In certain embodiments, the Fab specifically binds LRP5 and/or LRP6, and the VHH or sdAb (or scFv) specifically binds one or more Fzd receptor. In certain embodiments, the VHH or sdAb (or scFv) is fused to the N-terminus of the Fab, while in some embodiments, the VHH or sdAb (or scFv) is fused to the C-terminus of the Fab. In particular embodiments, the Fab is present in a full IgG format, and the VHH or sdAb (or scFv) is fused to the N-terminus and/or C-terminus of the IgG light chain. In particular embodiments, the Fab is present in a full IgG format, and the VHH or sdAb (or scFv) is fused to the N-terminus and/or C-terminus of the IgG heavy chain. In particular embodiments, two or more VHH or sdAb (or scFvs) are fused to the IgG at any combination of these locations.

Fabs may be converted into a full IgG format that includes both the Fab and Fe fragments, for example, using genetic engineering to generate a fusion polypeptide comprising the Fab fused to an Fc region, i.e., the Fab is present in a full IgG format. The Fc region for the full IgG format may be derived from any of a variety of different Fes, including but not limited to, a wild-type or modified IgG1, IgG2, IgG3, IgG4 or other isotype, e.g., wild-type or modified human IgG1, human IgG2, human IgG3, human IgG4, human IgG4Pro (comprising a mutation in core hinge region that prevents the formation of IgG4 half molecules), human IgA, human IgE, human IgM, or the modified IgG1 referred to as IgG1 LALAPG. The L235A, P329G (LALA-PG) variant has been shown to eliminate complement binding and fixation as well as Fc-γ dependent antibody-dependent cell-mediated cytotoxity (ADCC) in both murine IgG2a and human IgG1. These LALA-PG substitutions allow a more accurate translation of results generated with an “effectorless” antibody framework scaffold between mice and primates. In particular embodiments of any of the IgG disclosed herein, the IgG comprises one or more of the following amino acid substitutions: N297G, N297A, N297E, L234A, L235A, or P236G.

Non-limiting examples of bivalent and bispecific Engineered WNT agonists that are bivalent towards both the one or more Fzd receptor and the LRP5 and/or LRP6 are provided, including but not limited to those provided in Table 3. The VHH or sdAb (or scFvs) may be fused to the N-termini of both light chains, to the N-termini of both heavy chains, to the C-termini of both light chains, or to the C-termini of both heavy chains. It is further contemplated, e.g., that VHH or sdAb (or scFvs) could be fused to both the N-termini and C-termini of the heavy and/or light chains, to the N-termini of the light chains and the heavy chains, to the C-termini of the heavy and light chains, to the N-termini of the heavy chains and C-termini of the light chains, or to the C-termini of the heavy chains and the N-termini of the light chains. In other related embodiments, two or more VHH or sdAb (or scFvs) may be fused together, optionally via a linker moiety, and fused to the Fab or IgG at one or more of these locations. In a related embodiment, the Engineered WNT agonist has a Hetero-IgG format, whereas the Fab is present as a half antibody, and one or more VHH or sdAb (or scFv) is fused to one or more of the N-terminus of the Fc, the N-terminus of the Fab, the C-terminus of the Fc, or the C-terminus of the Fab. In certain embodiments, the Fab or antigen-binding fragment (or IgG) thereof is fused directly to the VHH or sdAb (or scFv) or antigen-binding fragment thereof, whereas in other embodiments, the binding regions are fused via a linker moiety.

In various embodiments, a Engineered WNT agonist comprises one or more Fab or antigen-binding fragment thereof that binds one or more FZD receptor and one or more Fab or antigen-binding fragment thereof that binds LRP5 and/or LRP6. In certain embodiments, it comprises two Fab or antigen-binding fragments thereof that bind one or more FZD receptor and/or two Fab or antigen-binding fragments thereof that bind LRP5 and/or LRP6. In particular embodiments, one or more of the Fab is present in a full IgG format, and in certain embodiments, both Fab are present in a full IgG format. In certain embodiments, the Fab in full IgG format specifically binds one or more FZD receptor, and the other Fab specifically binds LRP5 and/or LRP6. In certain embodiments, the Fab specifically binds one or more FZD receptor, and the Fab in full IgG format specifically binds LRP5 and/or LRP6. In certain embodiments, the Fab specifically binds LRP5 and/or LRP6, and the Fab in full IgG format specifically binds one or more FZD receptor. In certain embodiments, the Fab is fused to the N-terminus of the IgG, e.g., to the heavy chain or light chain N-terminus, optionally via a linker. In certain embodiments, the Fab is fused to the N-terminus of the heavy chain of the IgG and not fused to the light chain. In particular embodiments, the two heavy chains can be fused together directly or via a linker. In other related embodiments, two or more VHH or sdAb may be fused together, optionally via a linker moiety, and fused to the Fab or IgG at one or more of these locations. In a related embodiment, the Engineered WNT agonist has a Hetero-IgG format, whereas one of the Fab is present as a half antibody, and the other Fab is fused to one or more of the N-terminus of the Fc, the N-terminus of the Fab, or the C-terminus of the Fc. In certain embodiments, the Fab or antigen-binding fragment thereof is fused directly to the other Fab or IgG or antigen-binding fragment thereof, whereas in other embodiments, the binding regions are fused via a linker moiety.

In certain embodiments, the WNT agonists of the present invention can have, comprise, or consist of any of the sequences provided in in any of the Tables, Figures, or Examples herein, or functional fragments or variants thereof.

In certain embodiments, the FZD binding domain, LRP5/6 binding domain, and/or engineered WNT agonist binds with a dissociation constant (KD) of about 1 μM or less, about 100 nM or less, about 40 nM or less, about 20 nM or less, or about 10 nM or less. For example, in certain embodiments, a FZD binding domain or antibody described herein that binds to more than one FZD, binds to those FZDs with a KD of about 100 nM or less, about 20 nM or less, or about 10 nM or less. In certain embodiments, the binding domain binds to one or more its target antigen with an EC50 of about 1 μM or less, about 100 nM or less, about 40 nM or less, about 20 nM or less, about 10 nM or less, or about 1 nM 20 or less.

The engineered WNT agonists, binding domains thereof, antibodies or other agents of the present invention can be assayed for specific binding by any method known in the art. The immunoassays which can be used include, but are not limited to, competitive and non-competitive assay systems using techniques such as BIAcore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g., horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see e.g., Ausubel et al, eds, 1994, Current Protocols in Molecular Biology, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., FZD, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scatchard plot analysis. In some embodiments, BIAcore kinetic analysis is used to determine the binding on and off rates of antibodies or agents. BIAcore kinetic analysis comprises analyzing the binding and dissociation of antibodies from chips with immobilized antigens on their surface.

Engineered WNT agonists of the present invention are biologically active in binding to one or more FZD receptor and to one or more of LRP5 and LRP6, and in activation of WNT signaling. The term “WNT agonist activity” refers to the ability of an agonist to mimic the effect or activity of a WNT protein binding to a frizzled protein and/or LRP5 or LRP6. The ability of the engineered WNT agonists disclosed herein to mimic the activity of WNT can be confirmed by a number of assays. WNT agonists typically initiate a reaction or activity that is similar to or the same as that initiated by the receptor's natural ligand. In particular, the WNT agonists disclosed herein activate, enhance or increase the canonical WNT/β-catenin signaling pathway. As used herein, the term “enhances” refers to a measurable increase in the level of WNT/β-catenin signaling compared with the level in the absence of a WNT agonist, e.g., an engineered WNT agonist disclosed herein. In particular embodiments, the increase in the level of WNT/β-catenin signaling is at least 10%, at least 20%, at least 50%, at least 1.1-fold, at least 1.2-fold, at least 1.3-fold, at least 1.4-fold, at least 1.5-fold, at least two-fold, at least five-fold, at least 10-fold, at least 20-fold, at least 50-fold, or at least 100-fold as compared to the level of WNT/β-catenin signaling in the absence of the engineered WNT agonist, e.g., in the same cell type. Methods of measuring WNT/β-catenin signaling are known in the art and include those described herein.

In particular embodiments, engineered WNT agonists disclosed herein are bispecific, i.e., they specifically bind to two or more different epitopes, e.g., one or more FZD receptor, and LRP5 and/or LRP6. In certain embodiments, the engineered WNT agonists bind to FZD5 and/or FZD8, and LRP5 and/or LRP6.

In particular embodiments, engineered WNT agonists disclosed herein are multivalent, e.g., they comprise two or more regions that each specifically bind to the same epitope, e.g., two or more regions that bind to an epitope within one or more FZD receptor and/or two or more regions that bind to an epitope within LRP5 and/or LRP6. In particular embodiments, they comprise two or more regions that bind to an epitope within one or more FZD receptor and two or more regions that bind to an epitope within LRP5 and/or LRP6. In certain embodiments, engineered WNT agonists comprise a ratio of the number of regions that bind one or more FZD receptor to the number of regions that bind LRP5 and/or LRP6 of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, Engineered WNT agonists are bispecific and multivalent.

In certain aspects, the present disclosure provides novel tissue-specific WNT signal enhancing molecules capable of enhancing WNT activity in a tissue- or cell-specific manner. These may be used alone or in combination with one or more engineered WNT agonist disclosed herein. In certain embodiments, the tissue-specific WNT signal enhancing molecules are bi-functional molecules comprising a first domain that binds to one or more ZNRF3 and/or RNF43 ligases, and a second domain that binds to one or more targeted tissue or cell type in a tissue- or cell-specific manner. Each of the first domain and the second domain may be any moiety capable of binding to the ligase complex or targeted tissue or cell, respectively. For example, each of the first domain and the second domain may be, but are not limited to, a moiety selected from: a polypeptide (e.g., an antibody or antigen-binding fragment thereof or a peptide or polypeptide different from an antibody), a small molecule, and a natural ligand or a variant, fragment or derivative thereof. In certain embodiments, the natural ligand is a polypeptide, a small molecule, an ion, an amino acid, a lipid, or a sugar molecule. The first domain and the second domain may be the same type of moiety as each other, or they may be different types of moieties. In certain embodiments, the tissue-specific WNT signal enhancing molecules bind to a tissue- or cell-specific cell surface receptor. In particular embodiments, the tissue-specific WNT signal enhancing molecules increase or enhance WNT signaling by at least 50%, at least two-fold, at least three-fold, at least five-fold, at least ten-fold, at least twenty-fold, at least thirty-fold, at least forty-fold, or at least fifty-fold, e.g., as compared to a negative control.

Tissue-specific WNT signal enhancing molecules may have different formats. In particular embodiments, the tissue-specific WNT signal enhancing molecules are fusion proteins comprising a first polypeptide sequence that binds to ZNRF3/RNF43 and a second polypeptide sequence that binds to one or more targeted tissue or cell type in a tissue- or cell-specific manner. In certain embodiments, the two polypeptide sequences may be fused directly or via a linker. In certain embodiments, the tissue-specific WNT signal enhancing molecules comprise two or more polypeptides, such as dimers or multimers comprising two or more fusion proteins, each comprising the first domain and the second domain, wherein the two or more polypeptides are linked, e.g., through a linker moiety or via a bond between amino acid residues in each of the two or more polypeptides, e.g., an intermolecular disulfide bond between cysteine residues.

In particular embodiments, a tissue-specific WNT signal enhancing molecule is an antibody comprising antibody heavy and light chains (or antigen-binding fragments thereof) that constitute either the first domain or the second domain, wherein the other domain (i.e., the second domain or first domain) is linked to the antibody heavy chain or light chain, either as a fusion protein or via a linker moiety. In particular embodiments, the other domain is linked to the N-terminus of the heavy chain, the C-terminus of the heavy chain, the N-terminus of the light chain, or the C-terminus of the light chain. Such structures may be referred to herein as appended IgG scaffolds or formats. For example, a tissue-specific WNT signal enhancing molecule can be an antibody that binds ZNRF3/RNF43, wherein a binding domain that binds a tissue- or cell-specific receptor is fused or appended to either the heavy chain or light chain of the antibody that binds ZNRF3/RNF43. In another example, a tissue-specific WNT signal enhancing molecule can be an antibody that binds a tissue- or cell-specific receptor, wherein a binding domain that binds ZNRF3/RNF43 is fused or appended to either the heavy chain or light chain of the antibody that binds the tissue- or cell-specific receptor.

In particular embodiments, an intestine-specific WNT signal enhancing molecule is an antibody or antigen-binding fragment thereof that binds GPA33, CDH17, MUC-13, wherein a binding domain that binds ZNRF3/RNF43 is fused or appended to either the heavy chain or light chain of the antibody or antigen-binding fragment thereof. In particular embodiments, the binding domain that bind ZNRF3/RNF43 comprises Fu1 and Fu2 domains, wherein the Fu1 and Fu2 domains optionally comprise one or more amino acid modifications, including any of those disclosed herein, e.g., F105R and/or F109A.

In certain embodiments, the tissue-specific WNT signal enhancing molecules comprise a first domain (“action module”) that binds ZNRF3/RNF43 and a second domain (“targeting module”) that binds a tissue- or cell-specific receptor, e.g., with high affinity. In certain embodiments, each of these two domains has substantially reduced activity or is inactive in enhancing WNT signals by itself. However, when the tissue-specific WNT signal enhancing molecules engage with target tissues that express the tissue-specific receptor, E3 ligases ZNRF3/RNF43 are recruited to a ternary complex with the tissue-specific receptors, leading them to be sequestered, and/or cleared from the cell surface via receptor-mediated endocytosis. The net result is to enhance WNT signals in a tissue-specific manner.

In certain embodiments, the action module is a binder to ZNRF3/RNF43 E3 ligases, and it can be designed based on R-spondins, e.g., R-spondins-1-4, including but not limited to human R-spondins-1-4. In certain embodiments, the action module is an R-spondin, e.g., a wild-type R-spondin-1-4, optionally a human R-spondin-1-4, or a variant or fragment thereof. In particular embodiments, it is a variant of any of R-spondins-1-4 having at least 80%, at least 85%, at least 90%, at least 95%, at least 98%, or at least 99% sequence identity to the corresponding wild-type R-spondin-1-4 sequence. In certain embodiments, the action module comprises or consists of a Furin domain 1 of an R-spondin, e.g., any of R-spondins 1-4, which bind ZNRF3/RNF43. Extended versions of Furin domain 1 (including, but not limited to, those with a mutated Furin domain 2 that no longer binds to LGR4-6 or has reduced binding to LGR4-6) or engineered antibodies or any other derivatives or any engineered polypeptides different from antibodies that are able to bind specifically to ZNRF3/RNF43 can also be used. In certain embodiments, the action module comprises one or more Furin domain 1 of an R-spondin.

In certain embodiments, the action module does not comprise a Furin domain 2 of an R-spondin, or it comprises a modified or variant Furin domain 2 of an R-spondin, e.g., a Furin domain 2 with reduced activity as compared to the wild-type Furin domain 2. In certain embodiments, an action module comprises a Furin domain 1 but not a Furin domain 2 of R-spondin. In certain embodiments, an action module comprises two or more Furin domain 1 or multimers of a Furin domain 1. The action domain may comprise one or more wild-type Furin domain 1 of an R-spondin. In particular embodiments, the action module comprises a modified or variant Furin domain 1 of an R-spondin that has increased activity, e.g., binding to ZNRF3/RNF43, as compared to the wild-type Furin domain 1. Variants having increased binding to ZNRF3/RNF43 may be identified, e.g., by screening a phage or yeast display library comprising variants of an R-spondin Furin domain 1. Peptides or polypeptides unrelated to R-spondin Furin domain 1 but with increased binding to ZNRF3/RNF43 may also be identified through screening. Action modules may further comprise additional moieties or polypeptide sequences, e.g., additional amino acid residues to stabilize the structure of the action module or tissue-specific WNT signal enhancing molecule in which it is present.

In further embodiments, the action module comprises another inhibitory moiety, such as a nucleic acid molecule, which reduces or prevents ZNRF3/RNF43 activity or expression, such as, e.g., an anti-sense oligonucleotide; a small interfering RNA (siRNA); a short hairpin RNA (shRNA); a microRNA (miRNA); or a ribozyme. As used herein, “antisense” refers to a nucleic acid sequence, regardless of length, that is complementary to a nucleic acid sequence. In certain embodiments, antisense RNA refers to single-stranded RNA molecules that can be introduced to an individual cell, tissue, or subject and results in decreased expression of a target gene through mechanisms that do not necessarily rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA). In particular embodiments, the other inhibitor moiety inhibits an activity of one or both of ZNRF3/RNF43, or it inhibits the gene, mRNA or protein expression of one or both of ZNRF3/RNF43. In certain embodiments, the inhibitory moiety is a nucleic acid molecule that binds to a ZNRF3 RNF43 gene or mRNA, or a complement thereof.

In certain embodiments, the targeting module specifically binds to a cell-specific surface molecule, e.g., a cell-specific surface receptor, and can be, e.g., natural ligands, antibodies, or synthetic chemicals. In particular embodiments, the cell-specific surface molecule is preferentially expressed on a target organ, tissue or cell type, e.g., an organ, tissue or cell type in which it is desirous to enhance WNT signaling, e.g., to treat or prevent a disease or disorder. In particular embodiments, the cell-specific surface molecule has increased or enhanced expression on a target organ, tissue or cell type, e.g., an organ, tissue or cell type in which it is desirous to enhance WNT signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

Tissue-specific and cell-specific cell surface receptors are known in the art. Examples of tissue- and cell-specific surface receptors include but are not limited to GPA33, CDH17, and MUC-13. In certain embodiments, the targeting module comprises an antibody or antigen-binding fragment thereof that specifically binds these intestine specific receptors.

In certain embodiments, components of the engineered WNT agonist and WNT signal enhancing molecules may be combined to confer more tissue specificity.

The present invention is based, in part, upon the use of engineered WNT agonists to regulate gastrointestinal epithelium proliferation, in particular, in inflammatory bowel diseases.

In one embodiment, the present invention provides a method of treating a subject suffering from a gastrointestinal disorder comprising administering to the subject, an engineered WNT signaling modulator.

In certain embodiments, the engineered WNT agonist comprises one or more binding domains that bind to one or more FZD receptors (FZD1-10) and one or more binding domains that bind to one or more LRP (LRP5-6) receptors. In yet a further embodiment, the binding domains of the engineered WNT agonist comprise: one or more binding domains that bind to FZD5, FZD8, FZD1, FZD2, FZD7, FZD5,8, FZD1,2,7 or FZD1,2,7,5,8; FZD4; FZD9; or FZD10; and one or more binding domains that bind to LRP5, LRP6, or LRP5 and 6. In a further embodiment, the engineered WNT agonist comprises one or more binding domains that bind to FZD5 and/or FZD8; and one or more binding domains that bind to LRP5 and/or LRP6. In still a further embodiment the engineered WNT agonist comprises a binding domain that binds to FZD5 and FZD8, and a binding domain that binds LRP6. In further embodiments, the WNT agonist comprises a heavy chain sequence of SEQ ID NO: 1, 3, 5, 7, 9, 11, 13, 15, or 17 or a variable heavy chain region derived therefrom; and a light chain sequence of SEQ ID NO: 2, 4, 6, 8, 10, 12, 14, 16, or 18, or a variable light chain region derived therefrom.

In some embodiments, the engineered WNT agonist comprises a tissue targeting molecule. In a further embodiment, the tissue targeting molecule is an antibody or fragment thereof that binds to a tissue specific cell surface antigen. In some embodiments, the tissue targeting molecule is selected from the group consisting of Cell surface A33 antigen (GPA33; representative sequence is NCBI polypeptide reference sequence NP_005805.1), Cadherin-17 (CDH17; representative sequence is NCBI polypeptide reference sequence NP_004054.3), and Mucin 13 (cell surface associated (Muc-13; representative sequence is NCBI polypeptide reference sequence NP_149038.3), or a functional fragment or variant thereof. In certain embodiments, the WNT agonist is administered with a binding domain that specifically binds an inflammatory molecule. In further embodiments, the binding domain specifically binding the inflammatory molecule is an antagonist of the inflammatory molecule. In a further embodiment, the antagonist of the inflammatory molecule is an antagonist of TNFα, IL-12, IL-12 and IL-23, or IL-23.

In another related embodiment, the disclosure provides a combination molecule comprising: a) an engineered WNT agonist disclosed herein; and b) an engineered WNT signal enhancing molecule comprising a first domain that binds to one or more E3 ubiquitin ligases; and a second domain that binds to a tissue specific receptor.

In related embodiments, the disclosure provides a polypeptide that specifically binds Frizzed 5 (FZD5) and Frizzled 8 (FZD8), wherein the polypeptide comprises one or more sequence having at least 80%, at least 90%, at least 95%, or at least 98% homology to a sequence set forth in any of SEQ ID NOs: 1-18. In some embodiments, the polypeptide comprises an antibody or antibody binding fragment, e.g., one or more variable heavy chain or variable light chain. In some embodiments, said antibody or antibody binding fragment comprises at least 5 or all six of the CDRs present in any of the following combinations of sequence: SEQ ID NOs:1 and 2; SEQ ID NOs:3 and 4; SEQ ID NOs:5 and 6; or SEQ ID NOs:7 and 8, SEQ ID NOs:9 and 10, SEQ ID NOs: 11 and 12, SEQ ID NOs: 13 and 14, SEQ ID NOs:15 and 16, or SEQ ID NOs:17 and 18. In some embodiments, said polypeptide comprises six of the CDRs present in any of these combinations of sequences, wherein one or more of the CDRs comprises one, two, or three amino acid modifications, optionally a point mutation, an amino acid deletion, or an amino acid insertion.

The disclosure also provides a combination molecule comprising: an engineered WNT agonist disclosed herein; and an engineered WNT signal enhancing molecule comprising a first domain that binds to one or more E3 ubiquitin ligases; and a second domain that binds to a tissue specific receptor.

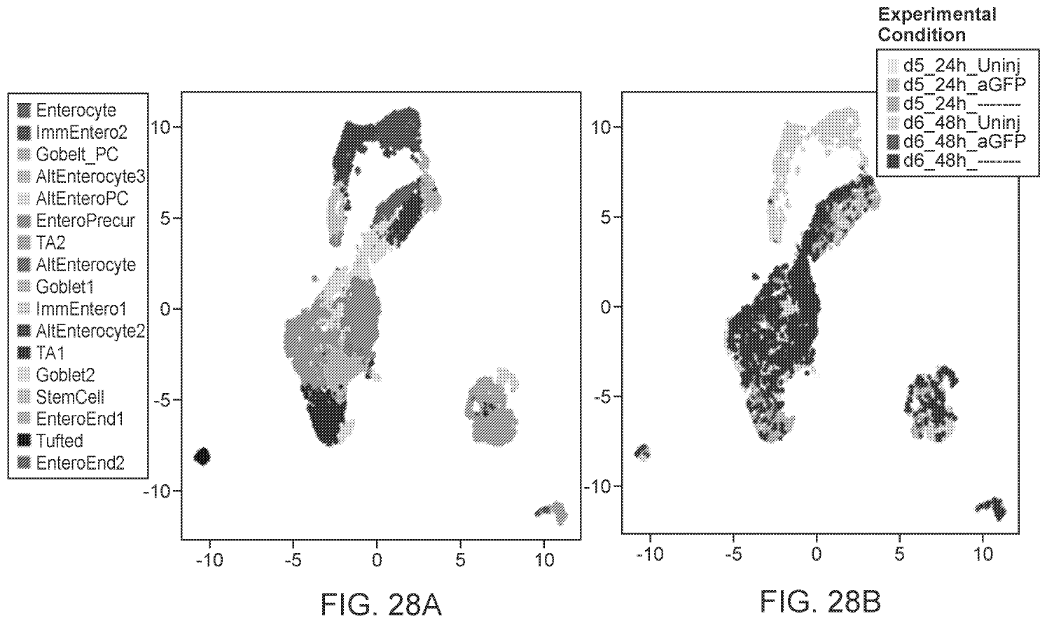

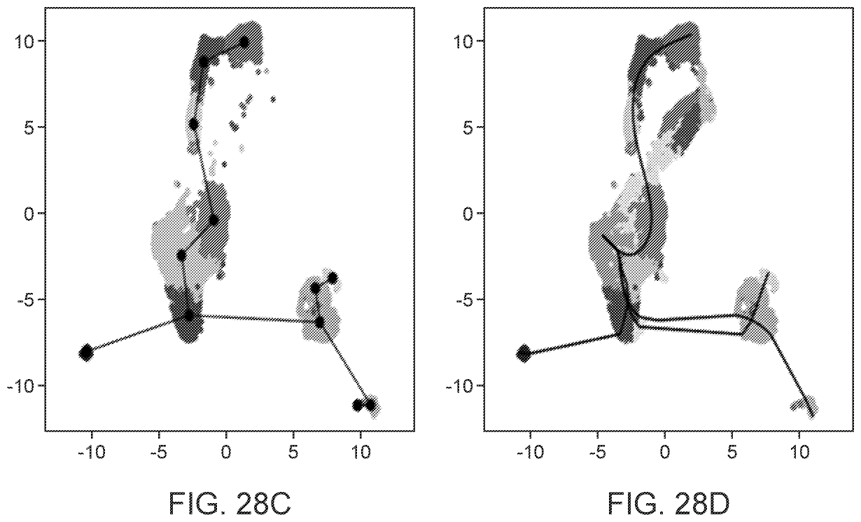

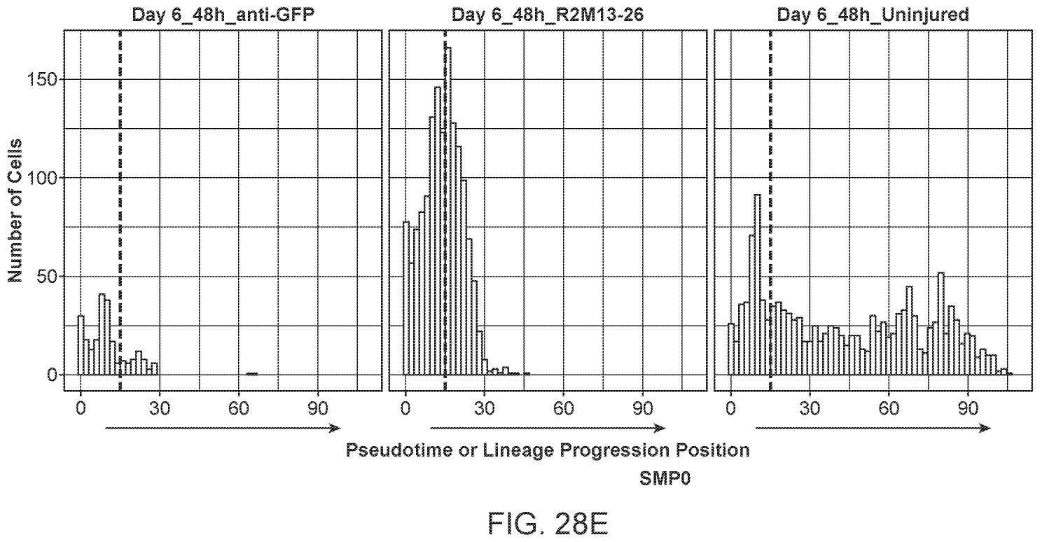

In some embodiments, an engineered WNT agonist of the disclosure promotes cell differentiation (e.g., gastrointestinal cells, stem cells, and/or epithelial cells) toward enterocytes. In some embodiments, cell differentiation is determined based on percentage of enterocyte precursors. In some embodiments, time-stamping-based methods are employed to determine cell differentiation toward enterocytes. In some embodiments, to complement time-stamp-based observations of cell differentiation, a lineage trajectory inference tool, slingshot, is employed. In some embodiments, slingshot predicts the direction of cell differentiation from an initial starting group. In an illustrative example, slingshot predicted cells would progress toward TA1, Goblet, Tufted, and enteroendocrine in one direction and toward enterocytes in the other directions. In some embodiments, predicted lineage trajectory pseudotime values show a higher percentage of engineered WNT agonist-treated samples that are further along in the enterocyte lineage trajectory relative to the control treated cells; FIG. 28E provides an illustrative example of predicted pseudotime values. In some embodiments, this prediction for the enterocyte lineage is congruent with the actual time-stamping data.

In some embodiments, a reliable standard for validating improved differentiation is the expression of mature, differentiated cell type markers looked more like that of naïve, uninjured colon in an engineered WNT agonist treatment group relative to the control groups on a given day after induced damage. In some embodiments, improved differentiation is observed 6-days after engineered WNT agonist treatment. In some embodiments, engineered WNT agonist treated samples include enterocytes, goblet cells, enteroendocrine, tuft cells, or a combination thereof.

Evidence disclosed herein indicates that Wnt mimetic molecules of the disclosure have desired properties, including the ability to restore diseased intestine tissue back to normal physiology. In some embodiments, short treatment using Wnt mimetics of the disclosure (e.g., R2M13-26 or R2M13-h26) induces rapid restoration of epithelial tissue. In an illustrative example, in a severe DSS model, a single injection of R2M13-26 at various doses restored normal histology of the damaged colon tissue. Within 6 days of treatment, R2M13-h26 completely restored the epithelial barrier, which was severely damaged in the acute DSS model.

Additional to barrier and colon tissue restoration, in some embodiments, treatment with Wnt mimetics of the disclosure reduces inflammatory cytokines and disease activity index, indicating elimination of the vicious cycle of barrier breach, microbial pathogen invasion, tissue inflammation and damage. In some embodiments, Wnt mimetics of the disclosure directly impact epithelial cells, expanding the progenitor pool and accelerating differentiation into all mature differentiated cell types. In some embodiments, Wnt mimetics of the disclosure restore Wnt signals and the stem cell niche in damaged colon tissue, without additional effects on the crypts after repair.

In some embodiments, treatment with Wnt mimetics of the disclosure alone does not have effects on normal intestine epithelium. In such embodiments, RSPO may induce hyperplasia. Taken together, the disclosure provides a Wnt activator with optimal tissue repair and physiological activities.

In an illustrative example, treatment with the Fzd5,8 specific Wnt mimetic, R2M13-26, resulted in rapid healing of the mucosa, improving tissue histology and disease activity in a few days with a concomitant reduction in inflammation and colitis symptoms. In this injury model, R2M13-26 predominately impacted the epithelium shortly after dosing. Wnt target genes such as Axin2 were increased in the epithelium at 24 hours post treatment, suggesting that utilizing FZD receptor specificity is a viable option for directing tissue-layer specific pathway activation. Coinciding with the induction of Wnt target genes, R2M13-26 caused a robust increase in cell cycle gene expression in a broad spectrum of progenitor cells, whether normal stem/progenitors responding to injury or in altered cell states consistent with de-differentiation. These transcriptome changes manifested in the transient expansion of the progenitor pool and accelerated differentiation into the proper secretory and absorptive lineages of the colonic epithelium and re-establishment of the epithelial barrier. This direct impact on epithelial regeneration and barrier restoration secondarily led to a reduction in inflammatory signals and infiltrating immune cells.

The injury/damage context may set the stage for epithelial progenitor expansion. As disclosed in the accompanying Examples, in addition to impacting developmental signaling pathways such as EGF and Notch, injury caused an inflammatory response in all tissue layers. In the epithelium, interferon gamma and NF-κB pathways were active after injury, and recent work in other stem cell niches has shown that inflammatory signaling can facilitate the initial proliferative response to injury (M. Chen, Reed, & Lane, 2017; Kyritsis et al., 2012). Activation of the NF-kB and Wnt pathways together may even promote the process of de-differentiation toward progenitors in the intestine (Schwitalla et al., 2013). In an illustrative example, in a DSS model of the disclosure, Wnt signaling was drastically reduced in the colonic epithelium possibly resulting from a reduction in expression of specific Wnts and an increase in several Wnt antagonists. In this illustrative example, R2M13-h26 was able to overcome this Wnt signaling deficiency. Thus, R2M13-h26-induced Wnt pathway activation may synergize with these inflammatory signals to enhance progenitor proliferation, albeit transiently.

Different from the effects of RSPO which impacts both the uninjured and damaged epithelium (Kim et al., 2005; Yan Kelley S. et al., 2017; Zhao et al., 2007), targeted, receptor-level Wnt signaling agonism with a Wnt mimetic of the disclosure may promote specific crypt proliferation in the damaged tissue context. In an illustrative example, extensive proliferation was observed in the small intestine and colon when the Wnt mimetic R2M13-26 was introduced together with RSPO2 in a DSS model. Although amelioration of DSS induced colitis in mice by RSPO was observed, hyperproliferation with RSPO treatment also occurred. The inventors surprisingly found that a Wnt mimetic by itself was able to induce expression of β-catenin target genes and proliferation of epithelial cells specifically in the injured colon.

In an illustrative example, Wnt pathway activation by R2M13-26 did not lead to crypt hyperproliferation or expansion. This stands in stark contrast to not only RSPO treatment but also the effects of heritable, genetic mutants. As previously reported, when the negative regulator Apc was genetically ablated or constitutively active mutants of Beta-catenin were expressed, crypts proliferated in an uncontrolled manner, failing to differentiate (Barker Nick et al., 2009; Krausova & Korinek, 2014; Mah, Yan, & Kuo, 2016). However, Wnt mimetics of the disclosure avoided these outcomes by mimicking endogenous Wnt signaling and initiating pathway activation at the receptor level, in contrast to the permanent genetic alterations that circumvent negative feedback. By impacting the pathway at the level of the receptor, R2M13-26 allowed negative feedback mechanisms to take effect. In this illustrative example, Axin2 was induced, contributing to the destruction complex; expression of the E3 ubiquitin ligase Rnf43, also a Wnt target gene, was increased, which promotes the removal of FZD receptors from the cell surface. Furthermore, in this illustrative example, R2M13-26 increased expression of some inhibitors of cyclin dependent kinases, potentially limiting proliferation.

### IV. Pharmaceutical Compositions

Pharmaceutical compositions comprising an engineered WNT agonist molecule described herein and one or more pharmaceutically acceptable diluent, carrier, or excipient are also disclosed. In another embodiment, the disclosure provides a pharmaceutical composition comprising a polypeptide, engineered WNT agonist, or combination molecule disclosed herein, and one or more pharmaceutically acceptable diluent, carrier, or excipient.

In further embodiments, pharmaceutical compositions comprising a polynucleotide comprising a nucleic acid sequence encoding a WNT agonist molecule (or a polypeptide chain thereof) described herein and one or more pharmaceutically acceptable diluent, carrier, or excipient are also disclosed. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences)).

In some embodiments the WNT agonist is an engineered recombinant polypeptide incorporating various epitope binding fragments that bind to various molecules in the WNT signaling pathway. For example, the FZD and LRP antibody fragments (e.g., Fab, scFv, sdAbs, VHH, etc.) may be joined together directly or with various size linkers, on one molecule.

Similarly, a polypeptide such as RSPO, may be engineered to contain an antibody or fragment thereof against a tissue specific cell surface antigen, e.g., MUC-13. RSPO may also be administered concurrently or sequentially with an enhancer of the E3 ligases, ZNRF3/RNF43. The E3 ligase enhancer may be an agonist antibody or fragment that binds ZNRF3/RNF43 and enhances the E3 ligase activity.

Conversely, WNT agonists can also be recombinant polypeptides incorporating epitope binding fragments that bind to various molecules in the WNT signaling pathway and enhance WNT signaling. For example, a WNT agonist can be an antibody or fragment thereof that binds to FZD receptor and/or an LRP receptor and enhances WNT signaling. The FZD and LRP antibody fragments (e.g., Fab, scFv, sdAbs or VHHs, etc) may be joined together directly or with various size linkers, on one molecule.

In further embodiments, pharmaceutical compositions comprising an expression vector, e.g., a viral vector, comprising a polynucleotide comprising a nucleic acid sequence encoding a WNT agonist molecule described herein and one or more pharmaceutically acceptable diluent, carrier, or excipient are also disclosed.

The present disclosure further contemplates a pharmaceutical composition comprising a cell comprising an expression vector comprising a polynucleotide comprising a promoter operatively linked to a nucleic acid encoding a WNT agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a WNT agonist. In particular embodiments, the cell is a heterologous cell or an autologous cell obtained from the subject to be treated.

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and may be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the engineered WNT agonist, e.g., an antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a WNT agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a WNT agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### V. Methods of Use

The present disclosure also provides methods for using the engineered WNT agonists and/or tissue-specific WNT signal enhancing molecules, e.g., to modulate a WNT signaling pathway, e.g., to increase WNT signaling, and the administration of an engineered WNT agonist and/or tissue-specific WNT signal enhancing molecule in a variety of therapeutic settings.

Also provided herein are methods of treatment using an engineered WNT agonist molecule and/or a tissue-specific WNT signal enhancing molecule.

In certain embodiments, an engineered WNT agonist may be used to increase Wnt signaling in a tissue or cell. Thus, in some aspects, the present invention provides a method for increasing Wnt signaling or enhancing Wnt signaling in a tissue or cell, comprising contacting the tissue or cell with an effective amount of an engineered WNT agonist or pharmaceutically acceptable salt thereof disclosed herein, wherein the an engineered WNT agonist is a Wnt signaling pathway agonist. In some embodiments, contacting occurs in vitro, ex vivo, or in vivo. In particular embodiments, the cell is a cultured cell, and the contacting occurs in vitro. In certain embodiments, the method comprises further contacting the tissue or cell with one or more Wnt polypeptides or Norrin polypeptides.

Engineered WNT agonists disclosed herein may be used in to treat a disease, disorder or condition, for example, by increasing Wnt signaling in a targeted cell, tissue or organ. Thus, in some aspects, the present invention provides a method for treating a disease or condition in a subject in need thereof, e.g., a disease or disorder associated with reduced Wnt signaling, or for which increased Wnt signaling would provide a therapeutic benefit, comprising contacting the subject with an effective amount of a composition of the present disclosure. In particular embodiments, the composition is a pharmaceutical composition comprising any of: an engineered WNT agonist; a polynucleotide comprising a nucleic acid sequence encoding an engineered WNT agonist, e.g., a DNA or mRNA, optionally a modified mRNA; a vector comprising a nucleic acid sequence encoding an engineered WNT agonist, e.g., an expression vector or viral vector; or a cell comprising a nucleic acid sequence encoding a an engineered WNT agonist. In particular embodiments, the disease or condition is a pathological disease or disorder, or an injury, e.g., an injury resulting from a wound. In certain embodiments, the wound may be the result of another therapeutic treatment. In certain embodiments, the disease or condition comprises impaired tissue repair, healing or regeneration, or would benefit from increased tissue repair, healing or regeneration. In some embodiments, contacting occurs in vivo, i.e., the subject composition is administered to a subject.

Wnt signaling plays key roles in the developmental process and maintenance of stem cells. Reactivation of Wnt signals is associated with regeneration and repair of most tissues after injuries and diseases. Engineered WNT agonist molecules are expected to provide benefit of healing and tissue repair in response to injuries and diseases. Causes of tissue damage and loss include but are not limited to aging, degeneration, hereditary conditions, infection and inflammation, traumatic injuries, toxins/metabolic-induced toxicities, or other pathological conditions. Wnt signals and enhancers of Wnt signals have been shown to activate adult, tissue-resident stem cells. In some embodiments, the compounds of the invention are administered for use in treating diseased or damaged tissue, for use in tissue regeneration and for use in cell growth and proliferation, and/or for use in tissue engineering.

Human diseases associated with mutations of the Wnt pathway provide strong evidence for enhancement of Wnt signals in the treatment and prevention of diseases. Preclinical in vivo and in vitro studies provide additional evidence of involvement of Wnt signals in many disease conditions and further support utilization of an engineered WNT agonist in various human diseases. For example, compositions of the present invention may be used to promote or increase bone growth or regeneration, bone grafting, healing of bone fractures, treatment of osteoporosis and osteoporotic fractures, spinal fusion, spinal cord injuries, including vertebral compression fractures, pre-operative spinal surgery optimization, osseointegration of orthopedic devices, tendon-bone integration, tooth growth and regeneration, dental implantation, periodontal diseases, maxillofacial reconstruction, and osteonecrosis of the jaw. They may also be used in the treatment of alopecia; enhancing regeneration of sensory organs, e.g. treatment of hearing loss, including regeneration of inner and outer auditory hair cells treatment of vestibular hypofunction, treatment of macular degeneration, treatment of retinopathies, including vitreoretinopathy, diabetic retinopathy, other diseases of retinal degeneration, Fuchs' dystrophy, other cornea disease, etc.; treatment of stroke, traumatic brain injury, Alzheimer's disease, multiple sclerosis, multiple dystrophy, muscle atrophy as a result of sarcopenia or cachexia, and other conditions affecting the degeneration or integrity of the blood brain barrier.

In certain embodiments, the present invention provides a method for treating a subject having a disease or disorder associated with reduced WNT signaling or for which increased Wnt signaling may be beneficial, comprising administering to the subject an effective amount of an engineered WNT agonist, or a pharmaceutical composition comprising an engineered WNT agonist. In certain embodiments, the disease or disorder is selected from the group consisting of: oral mucositis, short bowel syndrome, inflammatory bowel diseases (IBD), other gastrointestinal disorders, including, but not limited to: graft versus host disease (GVHD), alcoholic hepatitis, short bowel syndrome, celiac disease, radiation-induced gastro-intestinal mucositis and chemotherapy-induced gastro-intestinal mucositis; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; myocardial infarction, coronary artery disease, heart failure; immunodeficiencies, graft versus host diseases, acute kidney injuries, chronic kidney diseases, chronic obstructive pulmonary diseases (COPD), idiopathic pulmonary fibrosis (IPF), cirrhosis, acute liver failure, chronic liver diseases with hepatitis C or B virus infection or post-antiviral drug therapies, alcoholic liver diseases, alcoholic hepatitis, non-alcoholic liver diseases with steatosis or steatohepatitis, treatment of hearing loss, including internal and external loss of auditory hair cells, vestibular hypofunction, macular degeneration, treatment of vitreoretinopathy, diabetic retinopathy, other diseases of retinal degeneration, Fuchs' dystrophy, other corneal diseases, stroke, traumatic brain injury, Alzheimer's disease, multiple sclerosis and other conditions affecting the blood brain barrier; spinal cord injuries, bone related diseases, other spinal cord diseases, and alopecia.

The engineered WNT agonists and compositions of this invention may also be used in treatment of oral mucositis, treatment of short bowel syndrome, inflammatory bowel diseases (IBD), including Crohn's disease (CD) and ulcerative colitis (UC), in particular CD with fistula formation, other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; treatment of myocardial infarction, coronary artery disease, heart failure; enhanced growth of hematopoietic cells, e.g. enhancement of hematopoietic stem cell transplants from bone marrow, mobilized peripheral blood, treatment of immunodeficiencies, graft versus host diseases, etc.; treatment of acute kidney injuries, chronic kidney diseases; treatment of lung diseases, chronic obstructive pulmonary diseases (COPD), pulmonary fibrosis, including idiopathic pulmonary fibrosis, enhanced regeneration of lung tissues. The compositions of the present invention may also be used in enhanced regeneration of liver cells, e.g. liver regeneration, treatment of cirrhosis, enhancement of liver transplantations, treatment of acute liver failure, treatment of chronic liver diseases with hepatitis C or B virus infection or post-antiviral drug therapies, alcoholic liver diseases, including alcoholic hepatitis, non-alcoholic liver diseases with steatosis or steatohepatitis, and the like. The compositions of this invention may treat diseases and disorders including, without limitation, conditions in which regenerative cell growth is desired.

Human genetics involving loss-of-function or gain-of-function mutations in Wnt signaling components show strong evidence supporting enhancing Wnt signals for bone growth. Conditions in which enhanced bone growth is desired may include, without limitation, fractures, grafts, ingrowth around prosthetic devices, osteoporosis, osteoporotic fractures, spinal fusion, vertebral compression fractures, pre-operative optimization for spinal surgeries, osteonecrosis of the jaw, dental implantation, periodontal diseases, maxillofacial reconstruction, and the like. Engineered WNT agonists enhance and promotes Wnt signals which are critical in promoting bone regeneration. Methods for regeneration of bone tissues benefit from administration of the compounds of the invention, which can be systemic or localized. In some embodiments, bone marrow cells are exposed to molecules of the invention, such that stem cells within that marrow become activated.

In some embodiments, bone regeneration is enhanced by contacting a responsive cell population, e.g. bone marrow, bone progenitor cells, bone stem cells, etc. with an effective dose of an engineered WNT agonist disclosed herein. Methods for regeneration of bone tissues benefit from administration of the engineered WNT agonist which can be systemic or localized. In some such embodiments, the contacting is performed in vivo. In other such embodiments, the contacting is performed ex vivo. The molecule may be localized to the site of action, e.g. by loading onto a matrix, which is optionally biodegradable, and optionally provides for a sustained release of the active agent. Matrix carriers include, without limitation, absorbable collagen sponges, ceramics, hydrogels, polymeric microspheres, nanoparticles, bone cements, and the like.

In particular embodiments, compositions comprising one or more engineered WNT agonist disclosed herein (or a polynucleotide encoding an engineered WNT agonist, or a vector or cell comprising a polynucleotide encoding a Engineered WNT agonist) are used to treat or prevent a bone disease or disorder, including but not limited to any of the following, or to treat or prevent an injury associated with, but not limited to, any of the following: osteoporosis, osteoporotic fractures, bone fractures including vertebral compression fractures, non-union fractures, delayed union fractures, spinal fusion, osteonecrosis, osteonecrosis of the jaw, hip, femoral head, etc., osseointegration of implants (e.g., to accelerate recovery following partial or total knee or hip replacement), osteogenesis imperfecta, bone grafts, tendon repair, maxillofacial surgery, dental implant, all other bone disorders or defects resulting from genetic diseases, degeneration, aging, drugs, or injuries. In one embodiment, engineered WNT agonists that bind Fzd1, Fzd 2, and Fzd 7, and also LRP5 and/or LRP6, are used to treat or prevent any bone disease or disorder. In one embodiment, Engineered WNT agonists that bind Fzd1, Fzd 2, Fzd 5, Fzd 7 and Fzd 8, and also LRP5 and/or LRP6, are used to treat or prevent any bone disease or disorder. Other Fzd molecules that bind to additional Fzd receptors can also be used with LRP5 and/or LRP6 binders.

In particular embodiments, compositions and methods disclosed herein may be used to: increase bone mineral density, increase bone volume (e.g., tibia and/or femur bone volume), increase cortical thickness (e.g., in trabecular region or in femur mid-diaphysis), increase mineral apposition rate, increase the number of osteblasts and/or decrease the number of osteoclasts (e.g., in bone), increase bone stiffness, increase the ultimate load to fracture point, improve bone resistance to fracture, decrease bone resorption, decrease bone loss associated with osteoporosis, or increase biochemical strength of bone, in a subject. In one embodiment, engineered WNT agonists that bind Fzd1, Fzd 2, and Fzd 7 are used for any of these indicated uses. In one embodiment, engineered WNT agonists that bind Fzd1, Fzd 2, Fzd 5, Fzd 7 and Fzd 8 are used for any of these indicated uses.

Methods disclosed herein, including methods for treating or preventing a bone disease or disorder include methods that comprise providing to a subject in need thereof both an engineered WNT agonist and an antiresorptive agent. In certain embodiments, the methods are used for the treatment of osteoporosis, optionally post-menopausal osteoporosis.

The disclosure also provides a method for inhibiting or reducing bone resorption in a subject in need thereof, comprising providing to the subject an effective amount of an engineered WNT agonist, wherein the engineered WNT agonist is an agonist of a Wnt signaling pathway. In certain embodiments, the method further comprises providing to the subject an antiresorptive agent. In certain embodiments, the subject has been diagnosed with or is at risk for osteoporosis, optionally postmenopausal osteoporosis. A variety of antiresorptive agents are known in the art and include, but are not limited to, those disclosed herein.

When an engineered WNT agonist is provide to the subject in combination with another therapeutic agent, such as an antiresorptive agent, the two agent may be provided in the same or different pharmaceutical compositions. They may be provided to the subject at the same time, at different times, e.g., simultaneously, consecutively, or during overlapping or non-overlapping time periods. In certain embodiments, the two agents are therapeutically active in the subject during an overlapping time period.

Compositions comprising one or more engineered WNT agonist disclosed herein (or a polynucleotide encoding an engineered WNT agonist, or a vector or cell comprising a polynucleotide encoding an engineered WNT agonist) can be used for the in vivo treatment of skeletal tissue deficiencies. By “skeletal tissue deficiency”, it is meant a deficiency in bone or other skeletal connective tissue at any site where it is desired to restore the bone or connective tissue, no matter how the deficiency originated, e.g., whether as a result of surgical intervention, removal of tumor, ulceration, implant, fracture, or other traumatic or degenerative conditions. The compositions of the present invention can be used as part of a regimen for restoring cartilage function to a connective tissue, for the repair of defects or lesions in cartilage tissue such as degenerative wear and arthritis, trauma to the tissue, displacement of torn meniscus, meniscectomy, a luxation of a joint by a torn ligament, malalignment of joints, bone fracture, or by hereditary disease.

An engineered WNT agonist may also be used for treatment of periodontal diseases. Periodontal diseases are a leading cause of tooth loss and are linked to multiple systemic conditions. In some embodiments, tooth or underlying bone regeneration is enhanced by contacting a responsive cell population. In some such embodiments, the contacting is performed in vivo. In other such embodiments, the contacting is performed ex vivo, with subsequent implantation of the activated stem or progenitor cells. The molecule may be localized to the site of action, e.g., by loading onto a matrix, which is optionally biodegradable, and optionally provides for a sustained release of the active agent. Matrix carriers include, without limitation, absorbable collagen sponges, ceramics, hydrogels, bone cements, polymeric microspheres, nanoparticles, and the like.

Studies have shown that biology of Wnt signaling and R-spondins are capable of promoting sensory hair cell regeneration in the inner ear following injuries, aging, or degeneration. Loss of sensory hair cells in the inner ear involved in hearing loss or vestibular hypofunction may also benefit from the compositions of the invention. In the inner ear, the auditory organ houses mechanosensitive hair cells required for translating sound vibration to electric impulses. The vestibular organs, comprised of the semicircular canals (SSCs), the utricle, and the saccule, also contain sensory hair cells in order to detect head position and motion. Compositions of the present invention can be used, for example, in an infusion; in a matrix or other depot system; or other topical application to the ear for enhancement of auditory regeneration.

An engineered WNT agonist may also be used in regeneration of retinal tissue. In the adult mammalian retina, Muller glia cells are capable of regenerating retinal cells, including photoreceptors, for example after neurotoxic injury in vivo. Wnt signaling and enhancers of Wnt signals can promote proliferation of Muller glia-derived retinal progenitors after damage or during degeneration. The compositions of the invention may also be used in the regeneration of tissues and other cell types in the eye. For examples age-related macular degeneration (AMD), other retina degenerative diseases, cornea diseases, Fuchs' dystrophy, vitreoretinopathy, hereditary diseases, etc. can benefit from the compositions of the present inventions. AMD is characterized by progressively decreased central vision and visual acuity. Fuchs' dystrophy is characterized by progressive loss of cornea endothelial cells. Wnt signal and enhancing of Wnt signal can promote regeneration of cornea endothelium, retina epithelium, etc. in the eye tissue. In other embodiments, compositions of the present invention can be used, for example, in an infusion; in a matrix or other depot system; or other topical application to the eye for retinal regeneration and treatment of macular degeneration.

Specific populations of proliferating cells for homeostatic renewal of hepatocytes have been identified through lineage tracing studies, for example Axin2-positive cells in peri-central region. Lineage tracing studies also identified additional potential liver progenitor cells, including but not limited to Lgr-positive cells. The self-renewing liver cells and other populations of potential progenitor cells, including Lgr5-positive and Axin2-positive cells, are identified to be capable of regeneration responding to Wnt signals and/or R-spondins following injuries. Numerous preclinical models of acute liver injury and failure and chronic liver diseases showed recovery and regeneration of hepatocytes benefit from enhancing Wnt signals.

In certain embodiments, compositions comprising an engineered WNT agonist disclosed herein (or a polynucleotide encoding an engineered WNT agonist, or a vector or cell comprising a polynucleotide encoding an engineered WNT agonist) are used to promote liver regeneration, reduce fibrosis, and/or improve liver function. In certain embodiments, compositions and methods disclosed herein are used to: increase liver weight, increase the liver to body weight ratio, increase the number of PCNA and pH3 positive nuclei in liver, increase expression of Ki67 and/or Cyclin D1 in liver, increase liver cell proliferation and/or mitosis, decrease fibrosis following chronic liver injury, or increase hepatocyte function.

In particular embodiments, the compositions of this invention may be used in treatment of acute liver failure, acute alcoholic liver injuries, treatment of chronic liver diseases with hepatitis C or B virus infection or post-antiviral drug therapies, chronic alcoholic liver diseases, alcoholic hepatitis, non-alcoholic fatty liver diseases and non-alcoholic steatohepatitis (NASH), treatment of cirrhosis and severe chronic liver diseases of all causes, and enhanced regeneration of liver cells. Methods for regeneration of liver tissue benefit from administration of the compounds of the invention, which can be systemic or localized. These include, but are not limited to, methods of systemic administration and methods of localized administration e.g. by injection into the liver tissue, by injection into veins or blood vessels leading into the liver, by implantation of a sustained release formulation, and the like.

In particular embodiments, compositions comprising an engineered WNT agonist disclosed herein (or a polynucleotide encoding an engineered WNT agonist, or a vector or cell comprising a polynucleotide encoding an engineered WNT agonist) are used to treat or prevent a liver disease or disorder, including but not limited to, or to treat or prevent a liver injury or disorder resulting from any of the following: acute liver failure (all causes), chronic liver failure (all causes), cirrhosis, liver fibrosis (all causes), portal hypertension, alcoholic liver diseases including alcoholic hepatitis, nonalcoholic steatohepatisis (NASH), nonalcoholic fatty liver disease (NAFLD) (fatty liver), alcoholic hepatitis, hepatitis C virus-induced liver diseases (HCV), hepatitis B virus-induced liver diseases (HBV), other viral hepatitis (e.g., hepatitis A virus-induced liver diseases (HAV) and hepatitis D virus-induced liver diseases (HDV)), primary biliary cirrhosis, autoimmune hepatitis, livery surgery, liver injury, liver transplantation, “small for size” syndrome in liver surgery and transplantation, congenital liver disease and disorders, any other liver disorder or detect resulting from genetic diseases, degeneration, aging, drugs, or injuries.

Wnt signals play an important role in regeneration of various epithelial tissues. Various epidermal conditions benefit from treatment with the compounds of the present invention. Mucositis occurs when there is a breakdown of the rapidly divided epithelial cells lining the gastro-intestinal tract, leaving the mucosal tissue open to ulceration and infection. The part of the epithelial lining that covers the mouth, called the oral mucosa, is one of the most sensitive parts of the body and is particularly vulnerable to chemotherapy and radiation. Oral mucositis is probably the most common, debilitating complication of cancer treatments, particularly chemotherapy and radiation. In addition, the compositions of the invention may also benefit treatment of short bowel syndrome, inflammatory bowel diseases (JBD), or other gastrointestinal disorders. Other epidermal conditions include epidermal wound healing, diabetic foot ulcers, syndromes involving tooth, nail, or dermal hypoplasia, and the like. Molecules of the present invention may be used in all these conditions, where regenerative cells are contacted with compounds of the invention. Methods for regeneration of epithelial tissues benefit from administration of the compounds of the invention, which can be systemic or localized. Contacting can be, for example, topical, including intradermal, subdermal, in a gel, lotion, cream etc. applied at targeted site, etc.

In addition to skin and gastrointestinal tract, Wnt signals and enhancement and promotion of Wnt signals also play an important role in repair and regeneration of tissues including pancreas, kidney, and lung in preclinical models. An engineered WNT agonist may benefit various disease conditions involving exocrine and endocrine pancreas, kidney, or lung. The engineered WNT agonists may be used in treatment of metabolic syndrome; treatment of diabetes, treatment of acute or chronic pancreatitis, exocrine pancreatic insufficiency, treatment of acute kidney injuries, chronic kidney diseases, treatment of lung diseases, including but not limited to chronic obstructive pulmonary diseases (COPD), pulmonary fibrosis, in particular idiopathic pulmonary fibrosis (IPF), and other conditions that cause loss of lung epithelial tissues. Methods for regeneration of these tissues benefit from administration of the compounds of the invention, which can be systemic or localized.

Epidermal Wnt signaling, in coordination with signaling via other development factors, is critical for adult hair follicle regeneration. Hair loss is a common problem, and androgenetic alopecia, often called male pattern baldness, is the most common form of hair loss in men. In some embodiments, hair follicle regeneration is enhanced by contacting a responsive cell population with a molecule of the present invention. In some such embodiments, the contacting is performed in vivo. In other such embodiments, the contacting is performed ex vivo. The molecule may be localized to the site of action, e.g. topical lotions, gels, creams and the like.

Stroke, traumatic brain injury, Alzheimer's disease, multiple sclerosis and other conditions affecting the blood brain barrier (BBB) may be treated with an engineered WNT agonist. Angiogenesis is critical to ensure the supply of oxygen and nutrients to many tissues throughout the body, and is especially important for the CNS as the neural tissue is extremely sensitive to hypoxia and ischemia. CNS endothelial cells which form the BBB differ from endothelial cells in non-neural tissue, in that they are highly polarized cells held together by tight junctions and express specific transporters. Wnt signaling regulates CNS vessel formation and/or function. Conditions in which the BBB is compromised can benefit from administration of the compounds of the invention, which can be systemic or localized e.g. by direct injection, intrathecal administration, implantation of sustained release formulations, and the like. In addition, Wnt signal is actively involved in neurogenesis and plays a role of neuroprotection following injury. The compositions of the present invention may also be used in treatment of spinal cord injuries, other spinal cord diseases, stroke, traumatic brain injuries, etc.

Wnt signals also play a role in angiogenesis. An engineered WNT agonist may benefit conditions where angiogenesis is beneficial, treatment of myocardial infarction, coronary artery disease, heart failure, diabetic retinopathy, etc., and conditions from hereditary diseases. Methods for regeneration of these tissues benefit from administration of the compounds of the invention, which can be systemic or localized.

In certain embodiments, methods of the present invention promote tissue regeneration, e.g., in a tissue subjected to damage or tissue or cell reduction or loss. The loss or damage can be anything which causes the cell number to diminish, including diseases or injuries. For example, an accident, an autoimmune disorder, a therapeutic side-effect or a disease state could constitute trauma. Tissue regeneration increases the cell number within the tissue and preferably enables connections between cells of the tissue to be re-established, and more preferably the functionality of the tissue to be regained.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

In particular embodiments, a pharmaceutical composition is administered parenterally, e.g., intravenously, orally, rectally, or by injection. In some embodiments, it is administered locally, e.g., topically or intramuscularly. In some embodiments, a composition is administered to target tissues, e.g., to bone, joints, ear tissue, eye tissue, gastrointestinal tract, skin, a wound site or spinal cord. Methods of the invention may be practiced in vivo or ex vivo. In some embodiments, the contacting of a target cell or tissue with an engineered WNT agonist is performed ex vivo, with subsequent implantation of the cells or tissues, e.g., activated stem or progenitor cells, into the subject. The skilled artisan can determine an appropriate site of and route of administration based on the disease or disorder being treated.

The dose and dosage regimen may depend upon a variety of factors readily determined by a physician, such as the nature of the disease or disorder, the characteristics of the subject, and the subject's history. In particular embodiments, the amount of an engineered WNT agonist administered or provided to the subject is in the range of about 0.01 mg/kg to about 50 mg/kg, about 0.1 mg/kg to about 500 mg/kg, or about 0.1 mg/kg to about 50 mg/kg of the subject's body weight. In certain embodiments of any of the methods disclosed herein, the WNT agonist is administered to a subject, e.g., a mammal, intravenously, e.g., as a bolus injection, or subcutaneously. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 25 mg, about 75 mg, about 250 mg, about 750 mg, about 1500 mg or about 2250 mg of the WNT agonist. In particular embodiments, the subject is administered about 3 to about 30 mg/kg body weight intravenously or subcutaneously at least once per week of R2M13-h26, wherein R2M13-h26 comprises two polypeptides of SEQ ID NO:9 and two polypeptides of SEQ ID NO:10 bound by disulfide bonds.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent (e.g., a Engineered WNT agonist) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

In certain embodiments, following administration to a subject pf an engineered WNT agonist disclosed herein, the methods disclosed herein result in one or more of the following PK/PD parameters: Clearance (mL/day/kg) of 10-50 or about 25; terminal t1/2 of 2-5 days or about 4 days; Cmax (ug/mL) of 50 to 300 or 100 to 200, or about 140, MRT of about 3 to 4 (days), or about 4, or AUC (day*μg/mL) of about 100 to 1000 or about 100 to about 500, or about 190.

Other embodiments relate, in part, to the use of the engineered WNT agonists disclosed herein to promote or enhance the growth or proliferation of cells, tissues and organoids, for example, by contacting cells or tissue with one or more engineered WNT agonist, optionally in combination with a Norrin or Rspondin polypeptide. In certain embodiments, the cells or tissue are contacted ex vivo, in vitro, or in vivo. Such methods may be used to generate cells, tissue or organoids for therapeutic use, e.g., to be transplanted or grafted into a subject. They may also be used to generate cells, tissue or organoids for research use. The engineered WXNT agonists have widespread applications in non-therapeutic methods, for example in vitro research methods.

In certain embodiments, the engineered WNT agonists, including those disclosed herein, may be used to preserve cells, tissues, organs or organoids, e.g., tissue or organs for transplantation. For example, a cell, tissue, organ, or organoid may be contacted with an engineered WNT agonist in vivo or ex vivo. In the context of preserving cells, tissue, or organs for transplantation, the cell, tissue, organ, or organoid may be contacted with an engineered WNT agonist while still in the donor (i.e., before removal from the donor) and/or after removal from the donor. The methods may maintain or enhance viability of the cell, tissue, or organ, for example, during storage or prior to transplantation into a recipient. In particular embodiments, the cells, tissue, or organ is perfused in a composition or solution comprising the engineered WNT agonist. In certain embodiments, certain organ tissue is contacted with a WNT super agonist molecule to maintain viability of that tissue. In particular embodiments, the organ tissue is donor organ tissue to be transplanted to a recipient in need thereof. In certain embodiments, donor organ tissue is perfused in vivo with a solution comprising an engineered WNT agonist disclosed here, e.g., before the organ tissue is removed from the donor. In certain embodiments, donor organ tissue is perfused ex vivo with a solution comprising an engineered WNT agonist disclosed here, e.g., during storage or during transport from a donor to a recipient. In particular embodiment, the organ tissue contacted with an engineered WNT agonist remains viable for transplantation for at least 10%, at least 20%, at least 50%, or at least 100% longer than if it was not contacted with the engineered WNT agonist. In certain embodiments the organ tissue is liver tissue.

In certain embodiments, the engineered WNT agonists, including those disclosed herein, may be used for the expansion and/or maintenance of ex vivo tissue, e.g., skin tissue. In particular embodiments, the tissue is isolated from a donor or a patient. The tissue may be contacted with (e.g., maintained or cultured in the presence of) an engineered WNT agonist in vivo or ex vivo. In certain embodiments, the tissue is contacted ex vivo, e.g., by perfusion with a composition comprising an engineered WNT agonist.

In another embodiments, the engineered WNT agonists, including those disclosed herein, may be used to generate or maintain an organoid or organoid culture. For example, an organoid culture may be contacted with an engineered WNT agonist, for example, by culturing the organoid in a medium comprising an engineered WNT agonist. In certain embodiments, an organoid culture is generated, grown, or maintained by contacting it with one or more engineered WNT agonist disclosed herein. In particular embodiments, the engineered WNT agonist is present in the culture media used to grow or maintain the organoid tissue.

The invention provides a method for tissue regeneration of damaged tissue, such as the tissues discussed above, comprising administering an engineered WNT agonist to cells. The engineered WNT agonist may be administered directly to the cells in vivo, administered to a subject orally, intravenously, or by other methods known in the art, or administered to ex vivo cells. In some embodiments where the engineered WNT agonist is administered to ex vivo cells, these cells may be transplanted into a subject before, after or during administration of the engineered WNT agonist.

Wnt signaling is a key component of stem cell culture. For example, the stem cell culture media as described in WO2010/090513, WO2012/014076, Sato et al., 2011 (GASTROENTEROLOGY 2011; 141: 1762-1772) and Sato et at, 2009 (Nature 459, 262-5). The engineered WNT agonists disclosed herein are suitable alternatives to Rspondin for use in these stem cell culture media, or may be combined with Rspondin.

Accordingly, in one embodiment, the disclosure provides a method for enhancing the proliferation of stem cells comprising contacting stein cells with one or more Engineered WNT agonists disclosed herein. In one embodiment, the disclosure provides a cell culture medium comprising one or more engineered WNT agonists disclosed herein. In some embodiments, the cell culture medium may be any cell culture medium already known in the art that normally comprises Wnt or Rspondin, but wherein the Wnt or Rspondin is replaced (wholly or partially) or supplemented by engineered WNT agonist(s) disclosed herein. For example, the culture medium may be as described in as described in WO2010/090513, WO2012/014076, Sato et al., 2011 (GASTROENTEROLOGY 20J 1; 141: 1762-1772) and Sato et al, 2009 (Nature 459, 262-5), which are hereby incorporated by reference in their entirety.

Stem cell culture media often comprise additional growth factors. This method may thus additionally comprise supplying the stem cells with a growth factor. Growth factors commonly used in cell culture medium include epidermal growth factor (EGF, (Peprotech), Transforming Growth Factor-alpha (TGF-alpha, Peprotech), basic Fibroblast Growth Factor (bFGF, Peprotech), brain-derived neurotrophic factor (BDNF, R&D Systems), Hepatocyte Growth Factor (HGF) and Keratinocyte Growth Factor (KGF, Peprotech, also known as FGF7). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells. EGF or other mitogenic growth factors may thus be supplied to the stem cells. During culturing of stem cells, the mitogenic growth factor may be added to the culture medium every second day, while the culture medium is refreshed preferably every fourth day. In general, a mitogenic factor is selected from the groups consisting of: i) EGF, TGF-alpha, and KGF, ii) EGF, TGF-alpha, and FGF7; iii) EGF TGF-alpha, and FGF; iv) EGF and KGF: v) EGF and FGF7, vi) EGF and a FGF; vii) TGF-alpha and KGF; viii) TGF-alpha, and FGF7; ix) or from TG-alpha and a FGF. In certain embodiments, the disclosure includes a stem cell culture media comprising a Engineered WNT agonist disclosed herein, e.g., optionally in combination with one or more of the growth factors or combinations thereof described herein.

These methods of enhancing proliferation of stem cells can be used to grow new organoids and tissues from stein cells, as for example described in WO2010/090513 WO2012/014076, Sato et al., 2011 (GASTROENTEROLOGY 2011; 141: 1762-1772) and Sato et al., 2009 (Nature 459, 262-5).

In some embodiments, the engineered WNT agonists are used to enhance stem cell regeneration. Illustrative stem cells of interest include but are not limited to: muscle satellite cells; hematopoietic stem cells and progenitor cells derived therefrom (U.S. Pat. No. 5,061,620); neural stem cells (see Morrison et al (1999) Cell 96: 737-749); embryonic stem cells; mesenchymal stem cells; mesodermal stem cells; liver stem cells; adipose-tissue derived stem cells, etc.

The present invention is based, in part, upon the use of engineered WNT agonists to regulate gastrointestinal epithelium proliferation, in particular, in inflammatory bowel diseases.

In one embodiment, the present invention provides a method of treating a subject suffering from a gastrointestinal disorder comprising administering to the subject, an engineered WNT agonist disclosed herein. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC). In certain embodiments, the engineered WNT agonist reduces inflammatory cytokine expression in the intestine or colon and/or repairs intestinal epithelium.

In certain aspects, the present invention also provides a method of treating a subject suffering from a gastrointestinal disorder comprising administering to the subject a tissue-specific WNT signal enhancing molecule. In certain embodiments, the WNT signal enhancing molecule comprises: a) a first domain that binds to one or more E3 ubiquitin ligases; and b) a second domain that binds to a tissue specific receptor. In a further embodiment, the E3 ubiquitin ligases are selected from the group consisting of Zinc and Ring Finger Protein 3 (ZNRF3) and Ring Finger Protein 43 (RNF43). In another embodiment, the first domain comprises an R-spondin (RSPO) polypeptide. In a further embodiment, the RSPO polypeptide is selected from the group consisting of RSPO-1, RSPO-2, RSPO-3, and RSPO-4. In certain embodiments, the RSPO polypeptide comprises a first furin domain and a second furin domain. In certain embodiments, the second furin domain is wild-type or is mutated to have lower binding to Leucine-rich repeat-containing G protein coupled receptors 4-6 (LGR4-6). In certain embodiments, the engineered agonist or Wnt signal enhancing molecule incorporates a tissue targeting molecule. In further embodiments, the tissue targeting molecule is an antibody or fragment thereof that binds to a tissue specific cell surface antigen. In certain embodiments, the tissue targeting molecule is selected from the group consisting of GPA33, CDH17, and MUC-13, or a functional fragment or variant thereof. In some embodiments, the WNT agonist is administered with a binding domain that specifically binds an inflammatory molecule. In certain embodiments, the binding domain specific for the inflammatory molecule is an antagonist of the inflammatory molecule. In further embodiments, the antagonist of the inflammatory molecule is an antagonist of TNFα, IL-12, IL-12 and IL-23, or IL-23. In some embodiments, the gastrointestinal disease is inflammatory bowel disease. In further embodiments, the inflammatory bowel disease is selected from the group consisting of. Crohn's disease (CD), CD with fistula formation, and ulcerative colitis (UC).

In another embodiment, the present invention provides for a method of treating a subject suffering from a gastrointestinal disorder comprising administering to the subject an engineered WNT agonist and an engineered tissue specific WNT signal enhancing molecule. The engineered WNT agonist and the engineered tissue specific WNT signal enhancing molecule may be administered at the same time or at different times. In some embodiments, the subject comprises an effective amount of both during an overlapping time period. In certain embodiments, the engineered WNT agonist comprises one or more binding domains that bind to FZD5, FZD8, FZD1, FZD2, FZD7, FZD 5 and 8, or FZD1, 2, and 7, and one or more binding domains that bind to LRP5, LRP6, or LRP5. In some embodiments, the engineered WNT agonist comprises a tissue targeting molecule. In certain embodiments, the tissue targeting molecule is an antibody or fragment thereof that binds to a tissue specific cell surface antigen. In further embodiments, the tissue targeting molecule is selected from the group consisting of GPA33, CDH17, and MUC-13, or a functional fragment or variant thereof. In certain embodiments, the engineered WNT signal enhancing molecule comprises a first domain that binds to one or more E3 ubiquitin ligases, and a second domain that binds to a tissue specific receptor. In further embodiments, the E3 ubiquitin ligases are selected from the group consisting of Zinc and Ring Finger Protein 3 (ZNRF3) and Ring Finger Protein 43 (RNF43). In some embodiments, the first domain comprises an R-spondin (RSPO) polypeptide. In other embodiments, the RSPO polypeptide is selected from the group consisting of RSPO-1, RSPO-2, RSPO-3, and RSPO-4. In a further embodiment, the RSPO polypeptide comprises a first furin domain and a second furin domain. In yet a further embodiment, the second furin domain is wild-type or is mutated to have lower binding to Leucine-rich repeat-containing G protein coupled receptors 4-6 (LGR4-6). In further embodiments, the engineered WNT agonist is disclosed in Table 3. In some embodiments, the engineered WNT agonist and the engineered tissue specific WNT signal enhancing molecule are administered with a binding domain that specifically binds an inflammatory molecule. In further embodiments, the binding domain specific for the inflammatory molecule is an antagonist of the inflammatory molecule. In yet further embodiments, the antagonist of the inflammatory molecule is an antagonist of TNFα, IL-12, IL-12 and IL-23, or IL-23. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

In another embodiment, the present invention provides for a method of treating a subject suffering from a gastrointestinal disorder comprising administering to the subject, an engineered WNT agonist and an engineered tissue specific WNT signal enhancing combination molecule. In certain embodiments, the combination molecule comprises: a) the engineered WNT agonist comprising one or more binding domains that bind to FZD5, FZD8, FZD1, FZD2, FZD7, FZD 5 and 8, or FZD1, 2, and 7, and one or more binding domains that bind to LRP5, LRP6, or LRP5 and b) the engineered WNT signal enhancing molecule comprising a first domain that binds to one or more E3 ubiquitin ligases, and a second domain that binds to a tissue specific receptor. In further embodiments, the E3 ubiquitin ligases are selected from the group consisting of Zinc and Ring Finger Protein 3 (ZNRF3) and Ring Finger Protein 43 (RNF43). In some embodiments, the first domain comprises an R-spondin (RSPO) polypeptide. In other embodiments, the RSPO polypeptide is selected from the group consisting of RSPO-1, RSPO-2, RSPO-3, and RSPO-4. In a further embodiment, the RSPO polypeptide comprises a first furin domain and a second furin domain. In yet a further embodiment, the second furin domain is wild-type or is mutated to have lower binding to Leucine-rich repeat-containing G protein coupled receptors 4-6 (LGR4-6). In some embodiments, combination molecule incorporates a tissue targeting molecule. In certain embodiments, the tissue targeting molecule is an antibody or fragment thereof that binds to a tissue specific cell surface antigen. In further embodiments, the tissue targeting molecule is selected from the group consisting of GPA33, CDH17, and MUC-13, or a functional fragment or variant thereof. In some embodiments, the combination molecule is administered with a binding domain that specifically binds an inflammatory molecule. In further embodiments, the binding domain specific for the inflammatory molecule is an antagonist of the inflammatory molecule. In yet further embodiments, the antagonist of the inflammatory molecule is an antagonist of TNFα, IL-12, IL-12 and IL-23, or IL-23. In certain embodiments, the gastrointestinal disease is inflammatory bowel disease. In further embodiments, the inflammatory bowel disease is selected from the group consisting of: Crohn's disease (CD), CD with fistula formation, and ulcerative colitis (UC).

In particular embodiments of any of the methods disclosed herein, the engineered WNT agonist is selected from those disclosed in any of the following: PCT Application Publication No. WO 2016/040895; US Application Publication No. US 2017-0306029; US Application Publication No. US 2017-0349659; PCT Application Publication No. WO 2019/126398; or PCT Application Publication No. WO 2020/01030. In particular embodiments of any of the methods disclosed herein, the tissue-specific WNT signal enhancing molecule is selected from those disclosed in any of the following: PCT Application Publication No. WO 2018/140821; US Application Publication No. US 2020-0048324; or PCT Application Publication No. WO 2020/14271, all of which are herein incorporated by reference in their entireties.

In a related embodiment, the disclosure provides a method of treating a subject suffering from a gastrointestinal disorder comprising administering to the subject an engineered WNT agonist, an engineered WNT signal enhancing molecule, and/or a combination molecule disclosed herein, or a pharmaceutical composition comprising an engineered WNT agonist or combination molecule disclosed herein. In some embodiments, the gastrointestinal disorder is an inflammatory bowel disease, optionally selected from the group consisting of: Crohn's disease (CD), CD with fistula formation, and ulcerative colitis (UC). Any of the methods disclosed herein may be practiced using any of the engineered WNT agonists, engineered WNT signal enhancing molecules, and/or combination molecules disclosed herein.

In certain embodiments of any of the methods disclosed herein, the WNT agonist is administered to a subject, e.g., a mammal, intravenously, e.g., as a bolus injection. In particular embodiments, the WNT agonist is administered at least once per week. In particular embodiments, the subject is administered about 0.5 to about 100 mg/kg body weight of the WNT agonist, or about 2 to about 50 mg/kg body weight of the WNT agonist, e.g., about 2 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In particular embodiments, the subject is administered about 3 to about 30 mg/kg body weight intravenously at least once per week of R2M13-h26, wherein R2M13-h26 comprises two polypeptides of SEQ ID NO:9 and two polypeptides of SEQ ID NO:10 bound by disulfide bonds. In particular embodiments, the method is used to treat IBD, e.g., moderate to severe IBD with a WNT agonist disclosed herein, e.g., R2M13-h26. In certain embodiments, the IBD Crohn's disease, Crohn's disease with fistula formation, or ulcerative colitis.

Any of the methods disclosed herein may also be practiced using a combination of a WNT agonist molecule and a tissue-specific WNT signal enhancing molecule or a combination molecule comprising both a WNT agonist molecule and a tissue-specific WNT signal enhancing (combination molecule), e.g., as described herein. In one embodiment, a WNT agonist molecule and/or a tissue-specific WNT signal enhancing molecule, or combination molecule, is provided to a subject having a disease involving inappropriate or deregulated WNT signaling. In certain embodiments, methods disclosed herein comprise providing to a subject in need thereof a WNT agonist molecule and/or a tissue-specific WNT signal enhancing molecule, alone or in combination, or a combination molecule. In certain embodiments, a WNT agonist molecule and a tissue-specific WNT signal enhancing molecule are provided to the subject in the same or different pharmaceutical compositions. In some embodiments, the WNT agonist molecule and the tissue-specific WNT signal enhancing molecule are provided to the subject at the same time or at different times, e.g., either one before or after the other. In some embodiments, the methods comprise providing to the subject an effective amount of a WNT agonist molecule and/or tissue-specific WNT signal enhancing molecule. In some embodiments, an effective amount of the WNT agonist molecule and the tissue-specific WNT signal enhancing molecule are present in the subject during an overlapping time period, e.g., one day, two days, or one week. In other embodiments, methods disclosed herein comprise providing to a subject in need thereof a combination molecule comprising a WNT agonist molecule and a tissue-specific WNT signal enhancing molecule (combination molecule).

In certain embodiments, any of the methods disclosed herein may be practiced to reduce inflammation (e.g., inflammation associated with IBD or in a tissue affected by IBD, such as gastrointestinal tract tissue, e.g., small intestine, large intestine, or colon), increase WNT signaling, reduce any of the histological symptoms of IBD (e.g., those disclosed herein), reduce cytokine levels in inflamed tissue (e.g., gastrointestinal tract tissue), or reduce disease activity index as disclosed herein.

In certain embodiments, a WNT agonist molecule or tissue-specific WNT signal enhancing molecule or combination molecule may be used to enhance a WNT signaling pathway in a tissue or a cell. Agonizing the WNT signaling pathway may include, for example, increasing WNT signaling or enhancing WNT signaling in a tissue or cell. Thus, in some aspects, the present disclosure provides a method for agonizing a WNT signaling pathway in a cell, comprising contacting the tissue or cell with an effective amount of a WNT agonist molecule and/or a tissue-specific WNT signal enhancing molecule, or a combination molecule, or pharmaceutically acceptable salt thereof, disclosed herein, wherein the WNT agonist molecule and/or tissue-specific WNT signal enhancing molecule, or combination molecule is a WNT signaling pathway agonist. In certain embodiments, the disclosure provides a method of increasing WNT signaling in a cell, comprising contacting the cell with an engineered WNT agonist disclosed herein. In particular embodiments, the WNT agonist is R2M13-h26. In some embodiments, contacting occurs in vitro, ex vivo, or in vivo. In particular embodiments, the cell is a cultured cell, and the contacting occurs in vitro.

The WNT agonist and/or tissue-specific WNT signal enhancing molecule, or combination molecule may be used for the treatment of gastrointestinal disorders, including but not limited to, inflammatory bowel disease, including but not limited to, Crohn's disease, Crohn's disease with fistula formation, and ulcerative colitis. In particular embodiments, the WNT agonist may be used for the treatment of gastrointestinal disorders, including but not limited to, inflammatory bowel disease, including but not limited to, Crohn's disease with or without fistula formation, including but not limited to ulcerative colitis, including but not limited to acute intestinal GVHD (Graft versus host disease), including but not limited to Short Bowel Syndrome and any other gastro-intestinal disease where the epithelial barrier is impaired or the intestine is shortened. In particular the present invention provides a WNT/β-catenin signaling WNT/β-catenin agonist to enhance regeneration of the intestinal epithelium as a result of injury from these disorders. In particular embodiments, the WNT agonist is R2M13-h26.

The engineered WNT agonists may also be used to modulate a variety of tissue and/or cellular process, and to modulate gene expression within tissues and/or cell. In certain embodiments, the disclosure provides methods of modulating gene expression, comprising contact a subject, organ, tissue, or cells with an engineered WNT agonist disclosed herein, e.g., in Table 3. The subject may be administered the engineered WNT agonist, and the organ, tissue, or cells may be contacted with the engineered WNT agonist in vivo, ex vivo, or in vitro. In particular embodiments, the method results in upregulation or downregulation of one or more genes in the WNT signaling pathway, including but not limited to any of the genes disclosed in Tables 4-8. Upregulation or downregulation of gene expression may be measured at the RNA or protein level, and may result in an increase of at least two-fold, at least five-fold, at least 10-fold, or at least 20-fold or a decrease of at least 10%, 20%, 30%, 40%, 50%, 60%, 70%, 80%, or 90% in one or more tissues and/or cells of the subject following administration. The increase or decrease may be determined based on comparison to a pre-determined control level or the level determined for corresponding cells or tissue not contacted with the engineered WNT agonist, in certain embodiments.

In some embodiments, the disclosure provides a method of modulating expression of a WNT pathway molecule in one or more tissues and/or cells in a subject having a gastrointestinal disorder, comprising administering to the subject an engineered WNT agonist or the pharmaceutical composition disclosed herein. In certain embodiments, the WNT pathway molecule is a gene or protein listed in any one of Tables 4-7. In particular embodiments, the WNT pathway molecule is selected from the group consisting of: RNAse4, Angiongenin, Gsta3, Rnf43, Axin2, Ccnb1, or any of the genes or proteins listed in Table 7. In certain embodiments, expression of the WNT pathway molecule (gene or protein) is increased by at least 1.1-fold, at least 1.2-fold, at least 1.3-fold, at least 1.4-fold, at least 1.5-fold, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In certain embodiments, the tissue is epithelial tissue. In certain embodiments, the cells are gastrointestinal epithelial cells, optionally: stem cells, TA1, TA2, basal goblet, injury-induced alternative progenitors (AltEnteroPC), injury-induced alternative enterocytes (Alt Entero), enterocyte precursors (EnteroPrecur), goblet cells 1, goblet cells 2, enteroendocrine or tuft cells. In particular embodiments, the WNT agonist is R2M13-h26.

In another embodiment, the disclosure provides a method of stimulating tissue repair in a subject having a gastrointestinal disorder, comprising administering to the subject an engineered WNT agonist or the pharmaceutical composition disclosed herein. In particular embodiments, the tissue repair is stimulated by (or the method results in) modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4-8. In certain embodiments, the genes associated with the cell cycle are selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2. In certain embodiments, the genes associated with stem and progenitor cell renewal and differentiation are selected from those provided in Table 8, and Axin2, Id1, Hmga2, Nhp2, Foxq1, and Adh1. In certain embodiments, the genes associated with epithelial cell repair and barrier restoration are selected from those provided in Table 6, or Apex1, Agr2, B3gnt7, Fcgbp, Muc2, Muc3, Tff3, Zgl6, and Sprr2a3. In particular embodiments, expression of the gene is increased by at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In particular embodiments, the WNT agonist is R2M13-h26.

In another embodiment, the disclosure provides a method of reducing inflammation in a subject having a gastrointestinal disorder (or a tissue or cells thereof), comprising administering to the subject an engineered WNT agonist or the pharmaceutical composition disclosed herein. In certain embodiments, the inflammation is reduced by (or the method results in) modulation of at least one WNT pathway molecule selected from the group consisting of: genes provided in Table 5, or Adamdec1, Atf3, Gpx2, Gsta3, Gstm1, Gdf15, 1118, Nox1, Reg4, Sycn, Selenbp1, Tgfbr2, and Timp3. In particular embodiments, the inflammation is reduced in gastrointestinal tissue, optionally epithelial tissue. In certain embodiments, the inflammation is reduced in gastrointestinal epithelial cells, epithelial stem cells, TA1, TA2, basal goblet cells, injury-induced alternative progenitors (AltEnteroPC), injury-induced alternative enterocytes (AltEnteros), enterocyte precursors (EnteroPrecur), goblet cells 1, goblet cells 2, or enteroendocrine cells. In particular embodiments, expression of the WNT pathway molecule is increased by at least 1.1-fold, at least 1.2-fold, at least 1.3-fold, at least 1.4-fold, at least 1.5-fold, two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist. In particular embodiments, the WNT agonist is R2M13-h26.

In certain embodiments of any of the methods disclosed herein, the WNT agonist molecule may also incorporate a tissue targeting moiety, e.g., an antibody or fragment thereof that recognizes a pulmonary tissue specific receptor or cell surface molecule.

The present invention also provides for combination treatment with known and new treatments for gastrointestinal disorders, in particular inflammatory bowel diseases (IBD). For example, the WNT agonist can be combined with several known therapies for IBD, including, but not limited to, 5-Aminosalicylates (5-ASAs); immunosuppressants such as corticosteroids, azathioprine or 6-mercaptopurine, methotrexate, and ciclosporin-A or tacrolimus; TNFα inhibitors such as infliximab, adalimumab, and golimumab; anti-integrins such as vedolizumab; inflammatory cytokine antagonists such as ustekinumab; janus kinase (JAK) inhibitors such as tofacitinib; SMAD 7 inhibitors such as mongersen; and SIP modulators, such as ozanimod and etrasimod; and any new agents that may come on the market for the mentioned disorders. The above therapeutic drugs can be administered sequentially or concurrently with the molecules of the present invention.

The therapeutic agent (e.g., an engineered WNT agonist and/or tissue-specific WNT signal enhancing molecule or combination molecule) may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. In some embodiments, the subject method results in a therapeutic benefit, e.g., preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The scope of the invention is best understood with reference to the following examples, which are not intended to limit the inventions to the specific embodiments.

### EXAMPLES

### Example 1

### General Methods

Standard methods in molecular biology were utilized and are described, e.g., in the following: Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described, e.g., in Coligan et al. (2000) Current Protocols in Protein Science, Vol. 1, John Wiley and Sons, Inc., New York. Chemical analysis, chemical modification, post-translational modification, production of fusion proteins, glycosylation of proteins are described; see, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described, e.g., in Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available; see, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

Exemplary methods and materials used in the disclosure are provided below.

### RNA In Situ Hybridization:

Expression of mRNA was detected by RNAscope in situ hybridization (ACD Bio). RNAscope probes used are listed below. For colorimetric visualization, standard RNAscope® 2.5 HD Assay-Red protocol (www.acdbio.com) was followed, and images were acquired on a Leica DMi8 microscope equipped with a DFC7000T color camera. For fluorescent RNAscope in situ hybridization, standard RNAscope Multiplex Fluorescent Reagent Kit v2 Assay protocol was followed (ACD Bio Document #323100-USM) and coupled with the TSA Plus Cyanine 3 and 5 Systems. Fluorescent images were acquired with a Leica Thunder imaging system.

### RNA Isolation and RT-qPCR:

MagMAX™ mirVana (Thermofisher, A27828) Total RNA Isolation Kit was used for RNA isolation on a KingFisher (Thermofisher) sample purification system. Reverse Transcription was done with the Applied Biosystems High-Capacity cDNA Reverse Transcription Kit (Thermofisher, 4368814), and the Applied Biosystems TaqMan Fast Advanced Master Mix (Thermofisher, 4444557) was used for qPCR.

### Affinity Measurements:

Binding kinetics of R2M13, the Fzd binding portion of R2M13-26, Fab to each CRD of Fzd5,8 was determined by bio-layer interferometry (BLI) using Octet Red 96 (PALL ForteBio, Fremont, CA) instrument at 30° C., 1000 rpm with Streptavidin (SA) biosensors. Biotinylated CRDs of Fzds diluted to 25 nM in the running buffer (PBS, 0.05% Tween-20, 0.5% BSA, pH 7.2) were captured to the SA biosensor followed by dipping into wells containing the R2M13 Fab protein at different concentrations in running buffer or into a well with only running buffer as a reference channel. KD for each binder was calculated by Octet System software, based on fitting to a 1:1 binding model. Binding specificities of R2M13 IgG to 10 Fzds were also examined by the BLI assay. Biotinylated Fzd CRDs (H. Chen, Lu, Lee, & Li, 2020) diluted to 50 nM in the running buffer were captured to the SA biosensor followed by dipping into wells containing R2M13 IgG at 200 nM in running buffer.

### Super TopFlash (STF) Assay:

Signaling activity of the Wnt mimetics was measured using the Huh7 human liver cells containing a luciferase gene controlled by a WNT-responsive promoter (Super TopFlash reporter assay, STF) following an established protocol (H. Chen, Lu, Lee, & Li, 2020).

### Organoid Culture and Proliferation Assay:

Mouse small intestinal organoids were maintained in mouse IntestiCult™ Organoid Growth Medium (STEMCELL technologies) and passaged once a week until the date of assay for Wnt mimetic activity (H. Chen, Lu, Lee, & Li, 2020). To assay for organoid proliferation, organoids were dissociated with Gentle Cell Dissociation Reagent (STEMCELL technologies) for 10 min with shaking, washed 2× in cold PBS (Gibco) and resuspended 1:1 in Matrigel (Corning) on ice. 25 ul of cell resuspension in Matrigel was seeded to the center of each well on a prewarmed 48-well tissue culture plate and let solidify for 5 min at 37 degrees. 300 ul of Basal Media (Table 10), Basal Media+IWP2+anti-βGal or Basal Media+IWP2+Wnt mimetic was applied to the wells. Each condition included 5-6 repeats. Media and treatments were changed once on Day 4 after plating. Images of the 3D cultured organoids were acquired on Day 7.

### Animal Husbandry:

Seven-week-old C57Bl/6J female mice were obtained from Jackson Laboratories (Bar Harbor, ME, USA) and were housed 4-5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee. Mice were acclimatized a minimum of two days prior to initiating experiments. Mice were kept 12/12-hour light/dark cycle in a 30% to 70% humidity environment and room temperature ranging from 20° C. to 26° C.

### DSS Induced Acute Colitis:

7- to 8-week-old female C57BL6/J mice were fed with 4% (wt/vol) Dextran Sulfate Sodium (DSS, MP Biomedicals, molecular weight 36-50 kDa, Ref #160110) in drinking water from day 1 to day 7 to induce colitis and were switched to 1% DSS from day 8. Protein treatments were dosed either once on day 7 or twice on day 4 and 7. Animals were terminated on day 10, allowing a 6-day course of protein treatment, and the colon was harvested for histology and RT-qPCR. In one of the studies, DSS induced mouse body weight loss for animals treated with anti-GFP was nearly 25% on day 9 so animals were switched to drinking water with no DSS for compliance with IACUC rules. The disease activity index (DAI) was calculated based on the average score of weight loss, stool consistency and the degree of intestinal bleeding (Wirtz Stefan et al., 2017). Scoring system by grading was on a scale of 0 to 4 using the following parameters: loss of weight (0, 0-1%; 1, 1-6%; 2, 6-12%; 3, 12-18%; 4, >18%), stool consistency (0, normal; 1, soft but still formed; 2, soft; 3, very soft, wet; 4, watery diarrhea) and intestine bleeding (0-1, negative hemoccult; 2, positive hemoccult; 3, blood traces in stool visible; 4, gross rectal bleeding).

### Tissue Histology:

Small intestine and colon were extracted and, after removing fecal content, weighted and length measured. The desired small intestine segments (duodenum, jejunum, ileum) and colon segments (ascending, transverse and descending colon) were cut out and fixed directly in 10% neutral buffered formalin (NBF) overnight. Tissues were then transferred to 70% ethanol before paraffin embedding. Paraffin tissue blocks were then sectioned to 5 μM thickness and stained with hematoxylin and eosin (H&E) for histology analysis. Pathology reading was performed by an independent pathologist.

### Immunohistochemistry and Indirect Immunofluorescence:

In brief, five-micron thick formalin fixed paraffin embedded tissue sections on slides were deparaffinized followed by citrate buffer (pH 6) antigen retrieval in a steamer. Slides were then washed thoroughly in tap water followed by 1× wash in PBST. Subsequently, tissue sections were blocked with serum free protein block (Agilent, X090930-2) for one hour at room temperature followed by incubation in primary antibodies. After primary antibody incubation, tissue sections were washed in 0.1% TX-100 in PBS (PBST) at least three times followed by incubation in secondary antibody. Afterwards, tissue sections were washed with PBST, and coverslips were mounted with Vectashield Vibrance antifade mounting medium with DAPI (Vector Laboratories, H-1800).

### Fluorescence Activated Cell Sorting (FACS)

Mouse colon was dissociated as described below and resuspended in FACS buffer (HBSS, 2% FBS, 10 mM HEPES, 1 mM sodium pyruvate, and 1% Pen-strep or antibiotic/antimycotic solution). Prior to FACS, cells were passed through a 40-micron filter, and DAPI was added to distinguish live/dead cells. Prior to target antibody incubation, FcR blocking reagent (Miltenyi Biotec, 130-092-575) was added to the samples and incubated for 10 minutes.

Single Cell RNA-Sequencing (scRNA-Seq): Tissue Dissociation, Cell Isolation, Library Preparation, Sequencing

For the acute DSS model, mice were treated with 4% DSS in their drinking water throughout the duration of the experiment. DSS-treated animals were dosed with 10 mpk R2M13-26 or an anti-GFP antibody on day 4 of the DSS treatment. Cells from two uninjured, naïve mice (no DSS) at day 5 and day 6 and from three replicates each for anti-GFP and R2M13-26-treated DSS animals were collected for each timepoint. Each animal was considered a replicate.

Transverse colon was isolated from each animal and feces were removed. After a brief wash in cold PBS, the colon was cut longitudinally to open the tube into a flat sheet, and the tissue was cut into 3-4 mm length fragments. Tissue fragments were incubated in pre-warmed (37° C.) PBS with 5 mM EDTA in a shaker at 37° C. at 150 rpm for 15 minutes. After 15 minutes, the tubes containing the samples were vigorously shaken for 10 seconds to release more epithelial cells. The epithelial cells floating in suspension were removed to a new tube and centrifuged at 200 rcf for two minutes.

The residual tissue containing the remaining epithelia and stroma/lamina propria was then incubated in 8-12.5 mL of lamina propria dissociation buffer (AdvDMEM/F12 with 10 mM HEPES, 0.2% FBS, DNAsel (80 U/mL), Liberase™ (0.2 mg/mL), and 1% antibiotic/antimycotic) at 37° C. for 30 minutes with horizontal shaking at 150 rpm. After pelleting, the epithelial cells were resuspended in 1 mL of TrypLE with DNase1, and they were incubated at 37° C. for five minutes and triturated with a P1000 pipette for 30 seconds. After trituration, 10 mL of PBS plus 50 U/mL DNAsel were added to the epithelial cells, and they were centrifuged at 500 rcf, 4° C., and the supernatant was removed. Epithelial cells were then washed one time in FACS buffer (HBSS, 2% FBS, 10 mM HEPES, 1 mM sodium pyruvate, and 1% Pen-strep or antibiotic/antimycotic solution) before another round of centrifugation and final resuspension in 0.5 mL of FACS buffer. Following 30 minutes of dissociation in LP dissociation buffer, the remaining tissue fragments and suspension were centrifuged at 500 rcf for five minutes. Supernatant was removed down to 1 mL, and the sample was triturated with a P1000 until the solution was homogeneous and all tissue fragments had dissociated. After trituration, the sample was centrifuged at 500 ref for 5 minutes at 4° C. and washed in FACS buffer prior to resuspension in 1 mL of FACS buffer in preparation for FACS.

All cells were passed through a 40-micron filter prior to FACS. DAPI was used to assess viability by FACS, and only viable (DAPI-negative) cells were collected. A negative control without DAPI was used to ensure proper DAPI gating. Cells were collected from the epithelial fraction and then from the epithelial/lamina propria fraction and combined (1:5 ratio) and counted on a hemocytometer prior to cell capture. Standard 10× Genomics Chromium 3′ v3 scRNA-seq reagents (PN1000075) were used. Approximately 4000-4500 cells were loaded per channel. Cells from one, individual animal replicate were captured per channel. Standard 10× Genomics Chromium 3′ v3 scRNA-seq RT, cDNA amplification, and sequencing library preparation protocols were followed. Multiplexed sequencing libraries were sequenced on Illumina Nova Seq 6000 S1 lanes, averaging about 50,000 reads per cell.

scRNA-seq Analysis:

Illumina read data was processed using the 10× Genomics Cellranger (version 3.0.2) pipeline, which runs the STAR aligner, on the mm10-3.0.0 version of the mouse transcriptome. Demultiplexed UMI count data was then assessed, and following exploratory data analysis, low-quality cells and low expression genes were removed in part by using the R package scone (version 1.14.0) and data set specific filtering cut-offs: only cells with >1000 UMIs and with >=500 and <=6500 genes and less than or equal to 60000 UMIs were retained to remove presumably empty droplets and limit doublets. Cells with a mitochondrial gene percentage more than one standard deviation above the mean were filtered. Only those genes expressed in the upper quartile of at least three cells were obtained, yielding 16039 genes. UMI count data was normalized using deconvolution scaling from the R package scran (version 1.18.5; (Lun, Bach, & Marioni, 2016). After normalization, batch specific cell groups when assessed in reduced dimension space were not observed, nor were strong correlations between QC metrics and gene expression principal components observed.

To the complete and filtered data set, a shared nearest neighbor (SNN) graph-based clustering method (Xu & Su, 2015) was applied by using the wrapper function (buildSNNGraph) from the R package scran (version 1.18.5) with k equal to 40 coupled with the cluster_louvain function from the R package igraph (1.2.6) to the first 10 principal components derived from the top 2000 most variable genes across the data set. This allowed broad grouping of the cells and identification of cell types within the three tissue layers/lineages (immune, stromal, epithelial). Based on this initial clustering, the data were subsetted into these three smaller data sets, and the cells within each layer/lineage were clustered using the SNN graph-based method and the walktrap algorithm implemented with the cluster_walktrap function from the igraph package, applied to the first 15 principal components derived from the top 2000 most variable genes within that subsetted layer/lineage (immune, stromal, epithelial). Cell type/subtype identities were determined using established marker genes and published literature.

Differential gene expression analysis between experimental conditions was performed with the R package edgeR (version 3.32.1) (Y. Chen, Lun, & Smyth, 2016; Robinson, McCarthy, & Smyth, 2010) on pseudobulk samples following aggregation of single cells within biological replicate samples. This type of DE analysis was implemented at the lineage level and at the cell type/cluster level. Differential expression comparisons were performed between experimental conditions (DSS-injury versus uninjured and within the DSS-injury samples for R2M13-26-treated versus anti-GFP treated) within each of the three layers/lineages (epithelial, stromal, immune) and within individual cluster/cell types within each lineage for each timepoint (24-hours or 48-hours). Gene set enrichment analysis (GSEA), also called pathway analysis, was applied by implementing the fry function from the R package edgeR (version 3.32.1) (Y. Chen, Lun, & Smyth, 2016). Gene sets were obtained from the Broad Institute's Molecular Signature Database (MSigDB) and included Hallmark and curated (C2) gene sets of the KEGG, Biocarta, PID, Reactome, ST, SIG, an SA types. The kegga function of the edgeR package was also implemented, which only uses KEGG pathways, and similar results were observed (data not shown). To identify pathways that were differentially enriched in one experimental condition relative to another, GSEA was applied in both pairwise and more specific contrasts to the pseudobulk samples aggregated by replicate.

Lineage trajectory inference was performed using the R package slingshot (version 1.8.0; (Street et al., 2018).

To ascertain the ability of R2M13-26 to impact Wnt target gene expression, additional genes with support from the literature were added to a Wnt signaling target gene list (Gougelet et al., 2014), and differentially expressed genes by tissue layer and is presented in Table 7. Table 7 shows the Wnt target genes that were differentially expressed within the epithelial lineage when R2M13-26 treatment was compared to the anti-GFP treatment at either 24-hours or 48-hours. Differential expression was filtered on adjusted p-value (false discovery rate (FDR)) of <0.05.

### Example 2

### Engineered Wnt Agonists

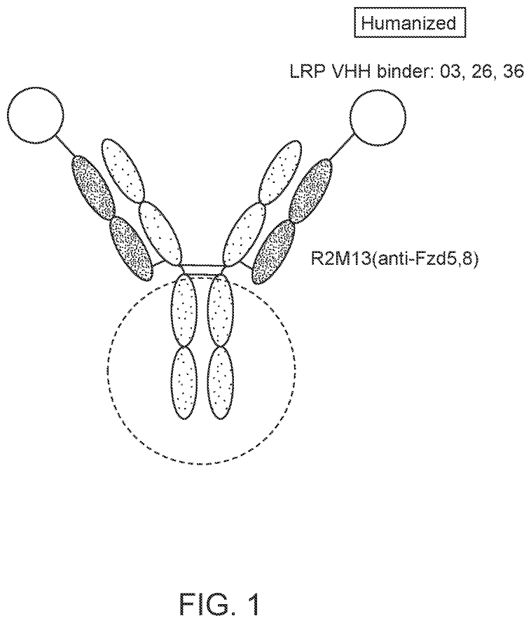

Engineered Wnt agonists in the IgG1 format were synthesized, including Wnt agonists having humanized Lrp5/6 binding domains fused to the N-terminus of each light chain of a Fzd binding antibody. An illustrative structure is shown in FIG. 1. The Lrp5/6 binding domain was derived from various camelid single chain antibody (VHH) binding domains selected from: VHH03, VHH26, or VHH36. The VHH03 domain binds Lrp5; the VHH26 domain binds Lrp6; and the VHH36 domain binds Lrp5 and Lrp6. The camelid single chain antibody was humanized by retaining the CDR sequences but replacing other sequences with a human antibody backbone. The resulting LRP5/6 binding domain was modified to remove potential liabilities.

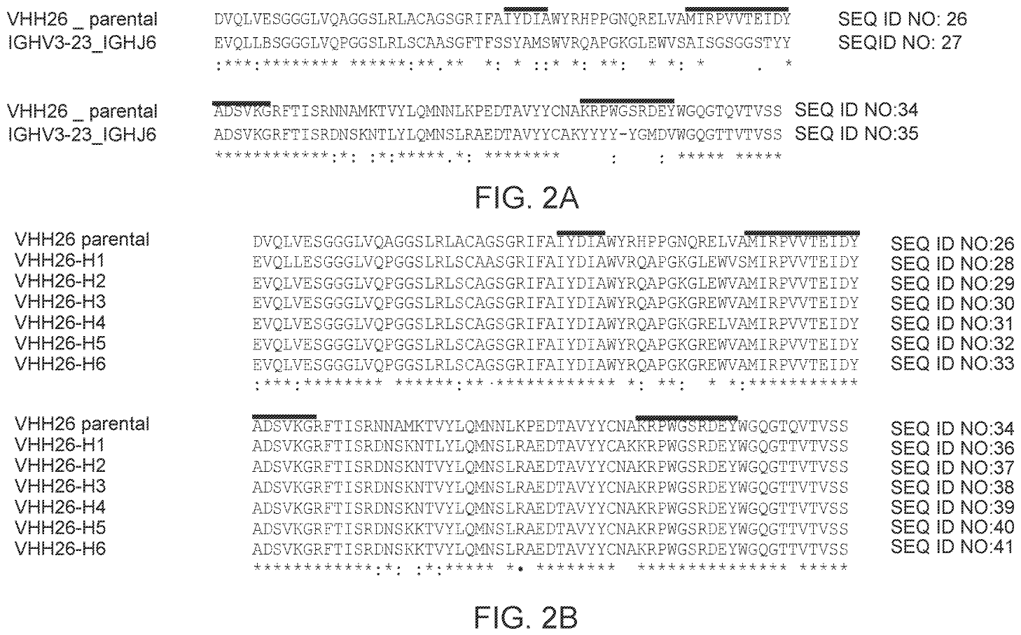

Humanization of VHH26 was performed as described below. Humanization of camelid VHH domains is considered to be challenging as they are derived from single-chain, homodimeric antibodies lacking VL:CL or VH:CH interactions present in hetrotetrameric human IgG1 antibodies. Surface properties of camelid VHHs (Muyldermans (2013) Annu. Rev. Biochem. 82:775-797; Vincke et al (2009) J. Biol. Chem. 284: 3273-3284) are evolutionarily reshaped to optimize the stability of homodimeric nature of single-chain antibodies. Humanization of camelid VHH26 was performed initially by CDR-grafting (for review: Safdari et al., (2013) Biotechnol. Genet. Eng. Rev. 27: 175-186) into a human germline sequence with highest sequence identity. In subsequent steps, several different humanized VHH26 constructs with back-mutations to camelid sequence were made to identify an engineered VHH with optimal expression, homogeneity and biophysical property such as binding affinity to the Lrp6 receptor. An alignment of VHH26 (Table 1) and its closest human germline sequence IGHV3-23*01 is shown in FIG. 2A. Sequences of six different humanized VHH26 (H1-H6) are listed in Table 1 and their alignment to the parental VHH26 is shown in FIG. 2B.

These six humanized VHH26 variants H1 to H6 and the parental VHH26 were transiently expressed in Expi293 cells (at 80 mL scale) with a C-terminal hexa-histidine tag. Proteins were purified using His-Complete resin (Roche, USA) following standard procedures. Expression levels and homogeneity of VHH26 and its humanized variants were analyzed by SDS-PAGE and SEC (size-exclusion chromatography). For Lrp6:VHH26 affinity determination, binding kinetics of VHH26-H1, VHH26-H2, VHH26-H3, VHH26-H4, VHH26-H5, VHH26-H6, and VHH26_His to biotinylated LRP6E3E4 (Chen et al., (2020) Cell Chemical Biol. 27, 1-12) were determined by bio-layer interferometry (BLI) using Octet Red 96 (PALL ForteBio, Fremont, CA) instrument at 30° C., 1000 rpm Streptavidin (SA) biosensors. Biotinylated LRP6E3E4 diluted to 50 nM in the running buffer (PBS, 0.05% Tween-20, 0.5% BSA, pH 7.2) were captured to the SA biosensor followed by dipping into wells containing the indicated VHH26 proteins at different concentrations in running buffer or into a well with only running buffer as a reference channel. KD for each binder was calculated by Octet System software, based on fitting to a 1:1 binding model. Kinetics values (Kon, Koff, KD) from each experiment was calculated from seven technical replicates of different concentrations of the molecule tested with Octet Data Analysis 9.0 (PALL ForteBio, Fremont, CA).

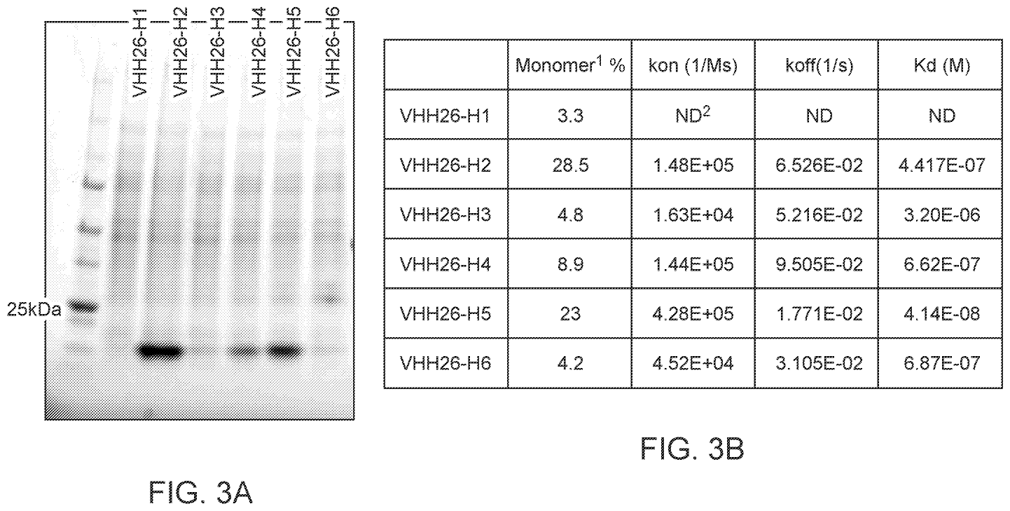

SDS-PAGE, SEC, and Octet-BLI profiles for VHH26 and its humanized variants are shown in FIGS. 3A-3B. Analyses of SDS-PAGE on Ni-pull-down samples reveal that among the six VHH26 human variants VHH26-H2, VHH26-H4, and VHH26-H5 showed higher level of expression compared to VHH26-H1, VHH26-H3, and VHH26-H6 (FIG. 3A). SEC analyses of all six humanized VHH26 constructs revealed two peaks and results are summarized in FIG. 3B. Central fractions of each of these peaks were examined by Octect-BLI for their ability interact with Lrp6. Kinetics parameters such as kon, koff, and KD for interaction between VHH26 constructs and Lrp6E3E4 domain are listed in Table 2. Analyses of these parameters reveal that the binding affinity towards Lrp6 is least affected in the case of the VHH26-H5 humanized variant compared to the parent VHH26 (Table 2; FIG. 3B). For comparison, alignment of parental VHH26 and VHH26-H5 is shown in FIG. 2B.

Based on the above, VHH26-H5 was used in the further experiments as a humanized LRP binding domain fused to a Fzd binding domain, e.g., tetravalent, bispecific WNT agonists. The Fzd binding domain was derived from the R2M13 antibody, which binds Fzd5 and Fzd8, and included an effector-less Fc region that retained FcRn binding, e.g., LALAPG or N297G (Wang X et al., Protein Cell 2018, 9:63-73). N297G is an aglycosylated form of IgG1 antibody, in which Asn is substituted by Gly. In the case of R2M13-26 humanized N297, the N297 corresponds to amino acid N302, so the N297G mutation may alternatively be referred to as N302G. LALAPG represents three mutations in the Fc domain of IgG1. Using a canonical IgG1 sequence numbering, Leu234 and Leu235 were mutated to Ala; similarly, Pro329 was mutated to Gly. Hence this triple-mutant in the Fc domain is referred to as “LALAPG”. In the case of R2M13-h26, these mutations are in sequence positions, 239, 240, and 334, respectively. VHH26-H5 was fused to the N-terminus of the light chains of the R2M13 antibody via a five amino acid linker, thus producing an IgG-like molecule comprising the R2M13 antibody with the VHH at the N-terminus of both antibody light chains.

Sequences of the R2M13 heavy chain IgG and the R2M13 light chain fused to the various LRP5/6 VHH binding domains by an amino acid linker present in the various Wnt agonists are provided in Table 3. The sequences of the heavy and light chains present in the parental R2M13-03, R2M13-26, R2M13-36 Wnt agonists without LALPG or N297G modifications are provided as SEQ ID NOs: 136-138 (light chains, respectively) and SEQ ID NO: 153 (heavy chains) in PCT Application Publication No. WO2019/126398, which is incorporated herein by reference in its entirety. The indicated Wnt agonist includes two of the heavy chains and two of the light chains in an antibody-type format, where the chains are connected via disulfide bonds.

TABLE 1
Sequences of parental and six humanized variants of VHH26

| Name | Sequence |
|---|---|
| VHH26_H1 | EVQLLESGGGLVQPGGSLRLSCAASGRIFAIYDIAWVRQAPGKGLEWV |
| (SEQ ID | SMIRPVVTEIDYADSVKGRFTISRDNSKNTLYLQMNSLRAEDTAVYYC |
| NO: 19) | AKKRPWGSRDEYWGQGTTVTVSS |
| VHH26_H2 | EVQLVESGGGLVQPGGSLRLSCAGSGRIFAIYDIAWYRQAPGKGLEWV |
| (SEQ ID | AMIRPVVTEIDYADSVKGRFTISRDNSKNTVYLQMNSLRAEDTAVYYC |
| NO: 20) | NAKRPWGSRDEYWGQGTTVTVSS |
| VHH26_H3 | EVQLVESGGGLVQPGGSLRLSCAGSGRIFAIYDIAWYRQAPGKGREWV |
| (SEQ ID | AMIRPVVTEIDYADSVKGRFTISRDNSKNTVYLQMNSLRAEDTAVYYC |
| NO: 21) | NAKRPWGSRDEYWGQGTTVTVSS |
| VHH26_H4 | EVQLVESGGGLVQPGGSLRLSCAGSGRIFAIYDIAWYRQAPGKGRELV |
| (SEQ ID | AMIRPVVTEIDYADSVKGRFTISRDNSKNTVYLQMNSLRAEDTAVYYC |
| NO: 22) | NAKRPWGSRDEYWGQGTTVTVSS |
| VHH26_H5 | EVQLVESGGGLVQPGGSLRLSCAGSGRIFAIYDIAWYRQAPGKGREWV |
| (SEQ ID | AMIRPVVTEIDYADSVKGRFTISRDNSKKTVYLQMNSLRAEDTAVYYC |
| NO: 23) | NAKRPWGSRDEYWGQGTTVTVSS |
| VHH26_H6 | EVQLVESGGGLVQPGGSLRLSCAGSGRIFAIYDIAWYRQAPGKGRELV |
| (SEQ ID | AMIRPVVTEIDYADSVKGRFTISRDNSKKTVYLQMNSLRAEDTAVYYC |
| NO: 24) | NAKRPWGSRDEYWGQGTTVTVSS |
| VHH26 | DVQLVESGGGLVQAGGSLRLACAGSGRIFAIYDIAWYRHPPGNQRELV |
| (parental) | AMIRPVVTEIDYADSVKGRFTISRNNAMKTVYLQMNNLKPEDTAVYYC |
| (SEQ ID | NAKRPWGSRDEYWGQGTQVTVSS |
| NO: 25) | |

TABLE 2
Kinetics parameters for interaction between
VHH26 constructs and Lrp6E3E4 domain

| Construct | SEC | | | KD |
|---|---|---|---|---|
| Name | Fraction | kon | koff | nM |
| VHH26-H1 | C3 | 1.42E+04 | 3.98E−03 | 281 |
| VHH26-H2 | G3 | 2.44E+05 | 6.84E−02 | 280 |
| VHH26-H3 | C3 | 1.84E+04 | 7.75E−03 | 422 |
| VHH26-H4 | G3 | 2.90E+04 | 5.56E−03 | 192 |
| VHH26-H5 | C3 | 1.35E+05 | 1.47E−02 | 109 |
| VHH26-H6 | G3 | 1.72E+04 | 8.36E−03 | 486 |
| VHH26 | D3 | 1.05E+06 | 1.82E−02 | 17 |
| (parental) | | | | |
| VHH26-H1 | C1 | ND | ND | ND |
| VHH26-H2 | G1 | 1.48E+05 | 6.53E−02 | 441 |
| VHH26-H3 | C1 | 1.63E+04 | 5.22E−02 | 3203 |
| VHH26-H4 | G1 | 1.44E+05 | 9.51E−02 | 662 |
| VHH26-H5 | C1 | 4.28E+05 | 1.77E−02 | 41 |
| VHH26-H6 | G1 | 4.52E+04 | 3.11E−02 | 687 |
| VHH26 | D3 | 8.10E+05 | 1.77E−02 | 22 |
| (parental) | | | | |

TABLE 3
Sequences of Wnt agonist heavy and light chains.

| Wnt | Heavy-chain (HC) and Light-chain (LC) sequences (LC of R2M13 antibody |
|---|---|
| agonist | underlined; linker in bold; VHH domain not bold or underlined)) |
| R2M13-03 | HC (SEQ ID NO: 1) |
| parental | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 2) |
| | DVQLVESGGGLVQPGGSLRLSCTSSANINSIETLGWYRQAPGKQRELIANMRG |
| | GGYMKYAGSLKGRFTMSTESAKNTMYLQMNSLKPEDTAVYYCYVKLRDDD |
| | YVYRGQGTQVTVSSGGSGSGSGDIQMTQSPSSLSASVGDRVTITCRASQSISS |
| | YLNWYQQKPGKAPKLLIYAASSLQSGVPSRFSGSGSGTDFTLTISSLQPEDFAT |
| | YYCQQSYSTPLTFGGGTKVEIKRTVAAPSVFIFPPSDEQLKSGTASVVCLLNNF |
| | YPREAKVQWKVDNALQSGNSQESVTEQDSKDSTYSLSSTLTLSKADYEKHKV |
| | YACEVTHQGLSSPVTKSFNRGEC |
| R2M13-03 | HC (SEQ ID NO: 3) |
| humanized | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 4) |
| | EVQLVESGGGLVQPGGSLRLSCASSANIQSIETLGWYRQAPGKQRELIANMRG |
| | GGYMKYADSLKGRFTMSTDNSKNTMYLQMNSLRAEDTAVYYCYVKLRDED |
| | YVYRGQGTQVTVSSGGGGSDIQMTQSPSSLSASVGDRVTITCRASQSISSYLN |
| | WYQQKPGKAPKLLIYAASSLQSGVPSRESGSGSGTDFTLTISSLQPEDFATYYC |
| | QQSYSTPLTFGGGTKVEIKRTVAAPSVFIFPPSDEQLKSGTASVVCLLNNFYPR |
| | EAKVQWKVDNALQSGNSQESVTEQDSKDSTYSLSSTLTLSKADYEKHKVYA |
| | CEVTHQGLSSPVTKSFNRGEC |
| R2M13-03 | HC (SEQ ID NO: 5) |
| humanized | EVQLLQSGAEVKKPGSSVKVSCKASGGTFTYRYLHWVRQAPGQGLEWMGGI |
| N297G | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 6) |
| | EVQLVESGGGLVQPGGSLRLSCASSANIQSIETLGWYRQAPGKQRELIANMRG |
| | GGYMKYADSLKGRFTMSTDNSKNTMYLQMNSLRAEDTAVYYCYVKLRDED |
| | YVYRGQGTQVTVSSGGGGSDIQMTQSPSSLSASVGDRVTITCRASQSISSYLN |
| | WYQQKPGKAPKLLIYAASSLQSGVPSRFSGSGSGTDFTLTISSLQPEDFATYYC |
| | QQSYSTPLTFGGGTKVEIKRTVAAPSVFIFPPSDEQLKSGTASVVCLLNNFYPR |
| | EAKVQWKVDNALQSGNSQESVTEQDSKDSTYSLSSTLTLSKADYEKHKVYA |
| | CEVTHQGLSSPVTKSFNRGEC |
| R2M13-26 | HC (SEQ ID NO: 7) |
| parental | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| (R2M13-26) | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 8) |
| | DVQLVESGGGLVQAGGSLRLACAGSGRIFAIYDIAWYRHPPGNQRELVAMIR |
| | PVVTEIDYADSVKGRFTISRNNAMKTVYLQMNNLKPEDTAVYYCNAKRPWG |
| | SRDEYWGQGTQVTVSSGSGSGDIQMTQSPSSLSASVGDRVTITCRASQSISSY |
| | LNWYQQKPGKAPKLLIYAASSLQSGVPSRFSGSGSGTDFTLTISSLQPEDFATY |
| | YCQQSYSTPLTFGGGTKVEIKRTVAAPSVFIFPPSDEQLKSGTASVVCLLNNFY |
| | PREAKVQWKVDNALQSGNSQESVTEQDSKDSTYSLSSTLTLSKADYEKHKVY |
| | ACEVTHQGLSSPVTKSFNRGEC |
| R2M13-26 | HC (SEQ ID NO: 9) |
| humanized | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| (R2M13-h26) | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 10) |
| | EVQLVESGGGLVQPGGSLRLSCAGSGRIFAIYDIAWYRQAPGKGREWVAMIR |
| | PVVTEIDYADSVKGRFTISRDNSKKTVYLQMNSLRAEDTAVYYCNAKRPWGS |
| | RDEYWGQGTTVTVSSGGGGSDIQMTQSPSSLSASVGDRVTITCRASQSISSYL |
| | NWYQQKPGKAPKLLIYAASSLQSGVPSRFSGSGSGTDFTLTISSLQPEDFATYY |
| | CQQSYSTPLTFGGGTKVEIKRTVAAPSVFIFPPSDEQLKSGTASVVCLLNNFYP |
| | REAKVQWKVDNALQSGNSQESVTEQDSKDSTYSLSSTLTLSKADYEKHKVY |
| | ACEVTHQGLSSPVTKSFNRGEC |
| R2M13-26 | HC (SEQ ID NO: 11) |
| humanized | EVQLLQSGAEVKKPGSSVKVSCKASGGTFTYRYLHWVRQAPGQGLEWMGGI |
| N297G | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 12) |
| | EVQLVESGGGLVQPGGSLRLSCAGSGRIFAIYDIAWYRQAPGKGREWVAMIR |
| | PVVTEIDYADSVKGRFTISRDNSKKTVYLQMNSLRAEDTAVYYCNAKRPWGS |
| | RDEYWGQGTTVTVSSGGGGSDIQMTQSPSSLSASVGDRVTITCRASQSISSYL |
| | NWYQQKPGKAPKLLIYAASSLQSGVPSRFSGSGSGTDFTLTISSLQPEDFATYY |
| | CQQSYSTPLTFGGGTKVEIKRTVAAPSVFIFPPSDEQLKSGTASVVCLLNNFYP |
| | REAKVQWKVDNALQSGNSQESVTEQDSKDSTYSLSSTLTLSKADYEKHKVY |
| | ACEVTHQGLSSPVTKSFNRGEC |
| R2M13-36 | HC (SEQ ID NO: 13) |
| parental | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 14) |
| | QVKLEESGGGLVQAGGSLRLSCAASGRIFSIYDMGWFRQAPGKEREFVSGIR |
| | WSGGTSYADSVKGRFTISKDNAKNTIYLQMNNLKAEDTAVYYCGSRGYWGQ |
| | GTLVTVSSGSGSGDIQMTQSPSSLSASVGDRVTITCRASQSISSYLNWYQQKP |
| | GKAPKLLIYAASSLQSGVPSRFSGSGSGTDFTLTISSLQPEDFATYYCQQSYSTP |
| | LTFGGGTKVEIKRTVAAPSVFIFPPSDEQLKSGTASVVCLLNNFYPREAKVQW |
| | KVDNALQSGNSQESVTEQDSKDSTYSLSSTLTLSKADYEKHKVYACEVTHQG |
| | LSSPVTKSFNRGEC |
| R2M13-36 | HC (SEQ ID NO: 15) |
| humanized | EVQLLQSGAEVKKPGSSVKVSCKASGGTFTYRYLHWVRQAPGQGLEWMGGI |
| LALAPG | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVL |
| | HQDWLNGKEYKCKVSNKALGAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 16) |
| | EVQLVESGGGLVQPGGSLRLSCAASGRIFSIYDMGWFRQAPGKEREFVSGIRW |
| | SGGTSYADSVKGRFTISKDNSKNTIYLQMNSLRAEDTAVYYCGSRGYWGQGT |
| | LVTVSSGGGGSDIQMTQSPSSLSASVGDRVTITCRASQSISSYLNWYQQKPGK |
| | APKLLIYAASSLQSGVPSRFSGSGSGTDFTLTISSLQPEDFATYYCQQSYSTPLT |
| | FGGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGLS |
| | SPVTKSFNRGEC |
| R2M13-36 | HC (SEQ ID NO: 17) |
| humanized | EVQLLQSGAEVKKPGSSVKVSCKASGGTFTYRYLHWVRQAPGQGLEWMGGI |
| N297G | IPIFGTGNYAQKFQGRVTITADESTSTAYMELSSLRSEDTAVYYCASSMVRVP |
| | YYYGMDVWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYGSTYRVVSVLTVL |
| | HQDWLNGKEYKCKVSNKALPAPIEKTISKAKGQPREPQVYTLPPSREEMTKN |
| | QVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | LC (SEQ ID NO: 18) |
| | EVQLVESGGGLVQPGGSLRLSCAASGRIFSIYDMGWFRQAPGKEREFVSGIRW |
| | SGGTSYADSVKGRFTISKDNSKNTIYLQMNSLRAEDTAVYYCGSRGYWGQGT |
| | LVTVSSGGGGSDIQMTQSPSSLSASVGDRVTITCRASQSISSYLNWYQQKPGK |
| | APKLLIYAASSLQSGVPSRFSGSGSGTDFTLTISSLQPEDFATYYCQQSYSTPLT |
| | FGGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGLS |
| | SPVTKSFNRGEC |

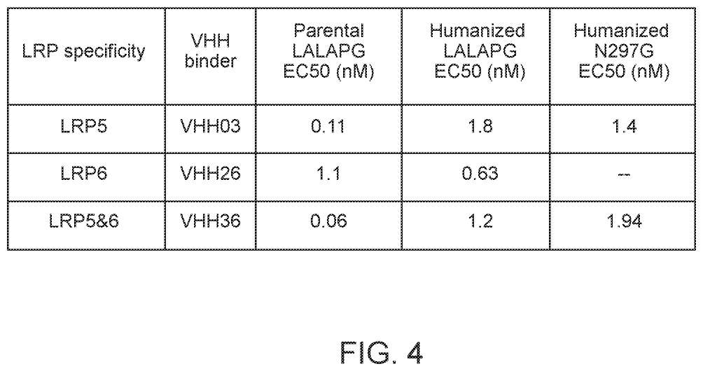

The activity of the Wnt agonists, with the Fzd binder R2M13 paired with various humanized Lrp binding domains in the context of the full engineered Wnt agonist format, was determined using the Super TOPFlash luciferase reporter (STF) assay, which measures activation of the canonical Wnt signaling in a Wnt responding Huh-7 reporter cell line (Huh-7STF). Results are shown in FIG. 4. The R2M13-humanized_26-LALAPG construct (“R2M13-26 humanized LALAPG”; also referred to herein as R2M13-h26, R2M13-h26-LALAPG, or humanized LALPG) showed the highest activity of the humanized Lrp binding domains. The R2M13-humanized_26-N297G construct (R2M13-26 humanized N297G; humanized N297G) was not stable. Humanization of VHH03 and VHH36, when paired with R2M13, reduced in vitro potency significantly, although their absolute EC50 values were comparable to VHH26 paired with R2M13. The sequences of the heavy and light chains of the R2M13-humanized_26-LALAPG construct (R2M13-h26) are shown in FIG. 6. The construct comprised two heavy chains and two light chains bound by disulfide bonds. The LALAPG mutations in the Fc domain removed effector function (see, e.g., Wang, et al. (2018) Protein Cell. 9:63-73). The various domains of the R2M13-h26 construct are shown, and the domains of the other constructs can be readily determined based on these.

### Example 3

### Dose Response of Engineered Wnt Agonists in an Animal Model of DSS Acute Colitis

The goal of this study was to examine efficacy of a Fzd5,8 specific Wnt mimetic, R2M13-26, disclosed in US Patent Application Publication No. 2020-0308287, and its dose response in the acute DSS colitis mouse model, characterize the in vivo activity of R2M13-26 with different dose and frequency, in the acute DSS colitis mouse model, and assess the impact of R2M13-26 on: 1) body weight, fecal score and occult blood, 2) epithelium/barrier repair by histology, and 3) inflammatory cytokine in serum and colon.

Six-eight-week old C57Bl/6J female mice (total of 86) were obtained from Jackson Laboratories (Bar Harbor, ME, USA) and were housed 5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

To induce acute colitis, 7- to 8-week-old female mice were given drinking water containing 4.0% (w/v) Dextran Sulfate Sodium (DSS, MP Biomedicals, MFCD00081551) ad libitum for 7 days followed by drinking water containing 100% (w/v) DSS for 3 days. Groups of mice were either untreated, treated with an isotype control antibody (anti-GFP), or treated with the indicated engineered Wnt agonist once on day 4 or twice on day 4 and 7.

R2M13-26 treatment once weekly at 1, 3, 10, 30 mg/kg, and twice weekly at 0.3, 1, 3, 10 mg/kg decreased disease activity index (DAI) in acute DSS mouse model. Single dose or twice weekly dose starting from 1 mg/kg of R2M13-26 was able to repair damaged colon epithelium, improving histology scores. Single dose or twice weekly dose starting from 1 mg/kg of R2M13-26 was able to decrease serum inflammatory cytokines, and colon cytokine levels.

This study confirmed that the Fzd5,8 specific Wnt mimetic (R2M13-26) alone was able to improve disease activity index, repair damaged colon epithelium, and decrease inflammatory cytokine levels in colon and serum in acute DSS mouse model. Overall, R2M13-26, with a wide dose range of treatment, improved fecal score and body weight, repaired damaged colon epithelium, and decreased inflammatory cytokine levels in the colon and in serum in the acute mouse IBD model (acute DSS).

### Example 4

### Engineered Wnt Agonists Repair Damaged Colon Epithelium in an Animal Model of DSS Acute Colitis

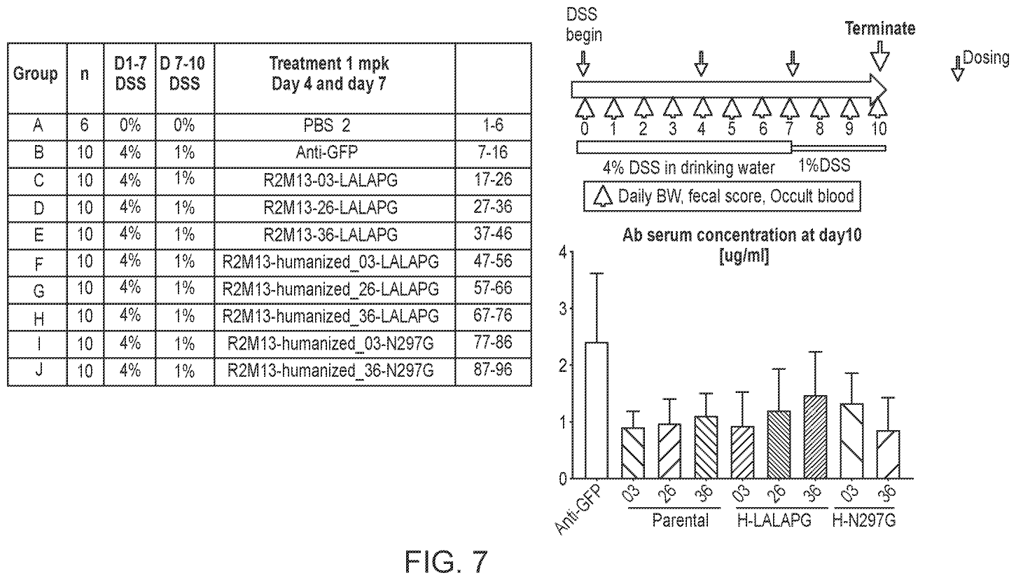

Various engineered humanized Wnt agonists were tested in the DSS model of acute colitis as outlined in FIG. 7. The constructs tested included non-humanized and humanized versions, including: R2M13-03-LALAPG, R2M13-26-LALAPG, R2M13-36-LALAPG, R2M13-humanized-03-LALAPG, R2M13-humanized-26-LALAPG, R2M13-humanized-36-LALAPG, R2M13-humanized-03-N297G, and R2M13-humanized-36-N297G.

Six-week old C57Bl/6J female mice (total of 96) were obtained from Jackson Laboratories (Bar Harbor, ME, USA) and were housed 5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee. Mice were acclimatized a minimum of two days prior to initiating experiments. Mice were kept 12/12-hour light/dark cycle in a 30% to 70% humidity environment and room temperature ranging from 20° C. to 26° C.

To induce acute colitis, 7- to 8-week-old female mice were given drinking water containing 4.0% (w/v) Dextran Sulfate Sodium (DSS, MP Biomedicals, MFCD00081551) ad libitum for 7 days followed by drinking water containing 1.0% (w/v) DSS for additional 3 days (FIG. 7). Groups of mice were either untreated, treated with an isotype control antibody (anti-GFP), or treated with 1 mg per kg of the indicated engineered Wnt agonist on day 4 and day 7. All protein treatments showed comparable serum antibody exposure at termination (FIG. 7).

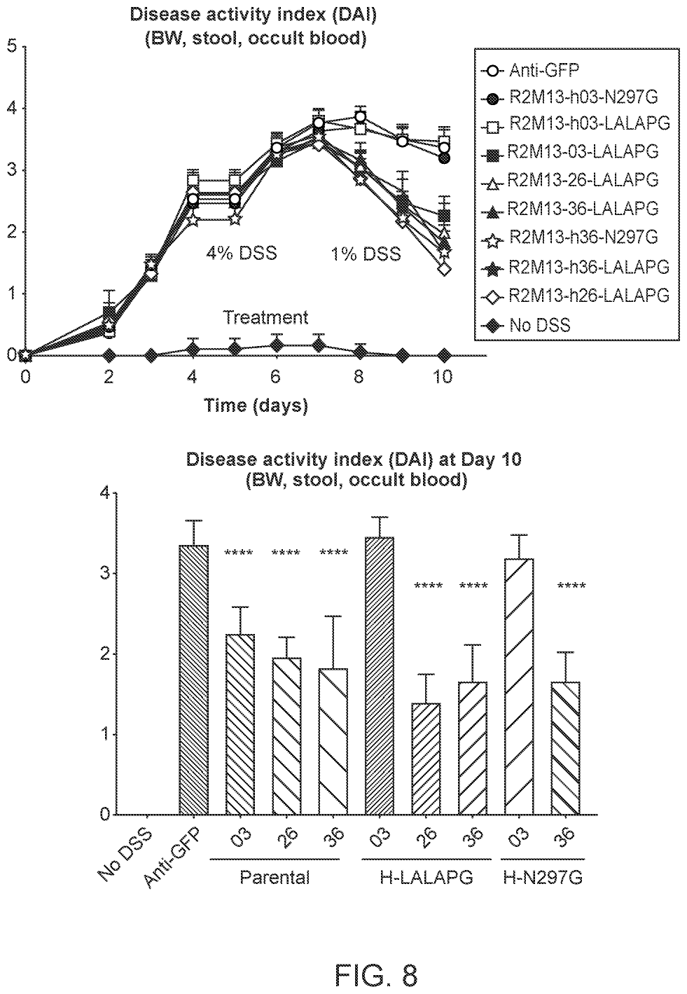

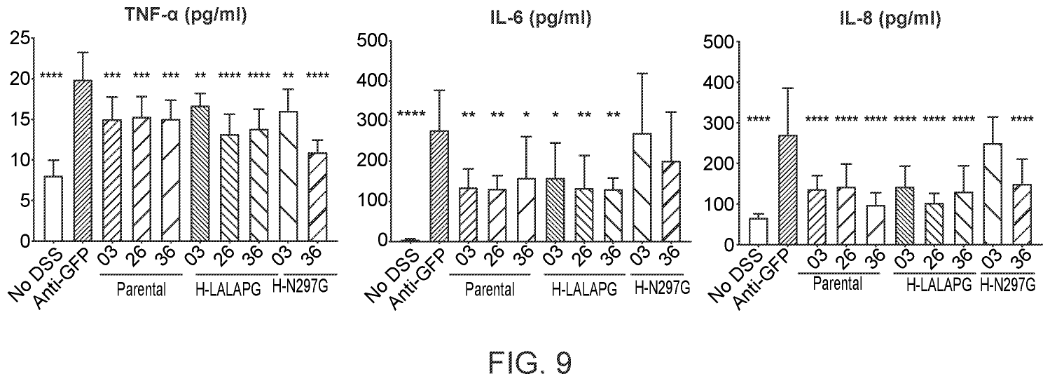

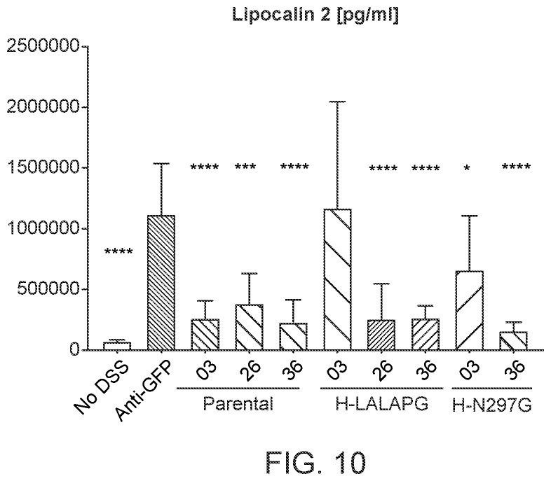

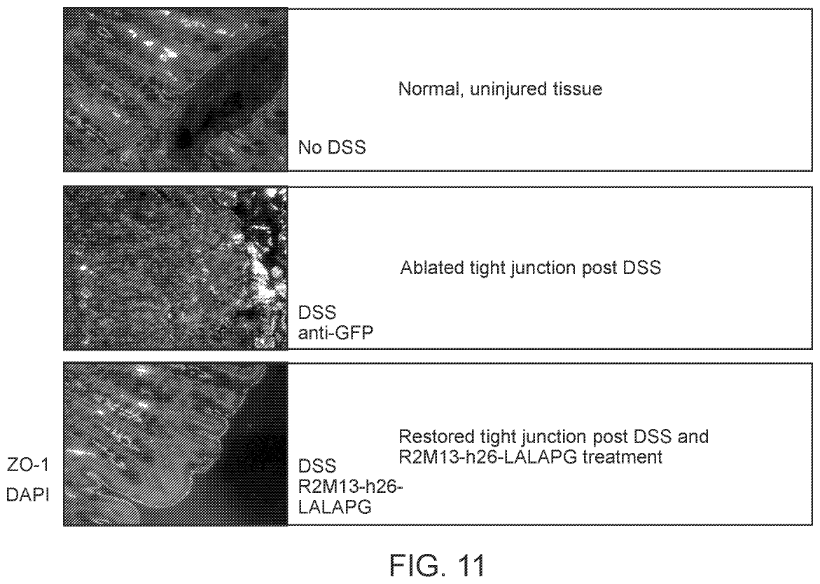

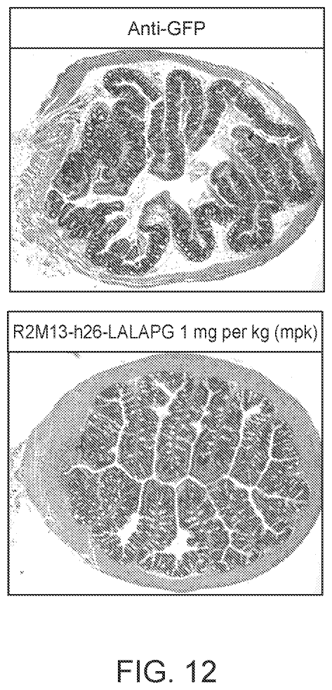

Control-treated animals subjected to DSS developed severe colitis characterized by profound and sustained weight loss and bloody diarrhea, resulting in the increase of disease activity index as represented by fecal score. Treatment with humanized R2M13-26 and humanized R2M13-36, either in the LALAPG or in the N297G form, significantly improved body weight in DSS mice. There was a significant improvement in body weight with the humanized R2M13-36-LALAPG as compared to the parental construct. These constructs also significantly decreased Disease Activity Index (DAI) in the DSS mice (FIG. 8); decreased fecal score in DSS mice; increased colon length and feces in DSS mice; and increased colon length and weight in DSS mice. Furthermore, humanized R2M13-26 (H-LALAPG 26) and humanized R2M13-36 (H-LALAPG 36) decreased serum level of inflammatory cytokines, tumor necrosis factor alpha (TNF-α), interleukin-6 (IL-6) and interleukin-8 (IL-8) (FIG. 9) and lipocalin-2, which were elevated in DSS-treated groups (FIG. 10). There was a significant improvement in body weight with the humanized R2M13-36-LALAPG as compared to the parental construct. Furthermore, humanized R2M13-26-LALAPG (R2M13-h26-LALAPG) was demonstrated to restore epithelial tight junction marker, ZO-1, in vivo (FIG. 11), repair damaged colon epithelium (FIG. 12), and restore the epithelial cell lineage including colonocytes, goblet cells, and tuft cells (FIG. 13). Thus, both humanized R2M13-26 and humanized R2M13-36 showed good efficacy in DSS mice.

### Example 5

### Pharmacokinetics (PK) of Engineered Wnt Agonists

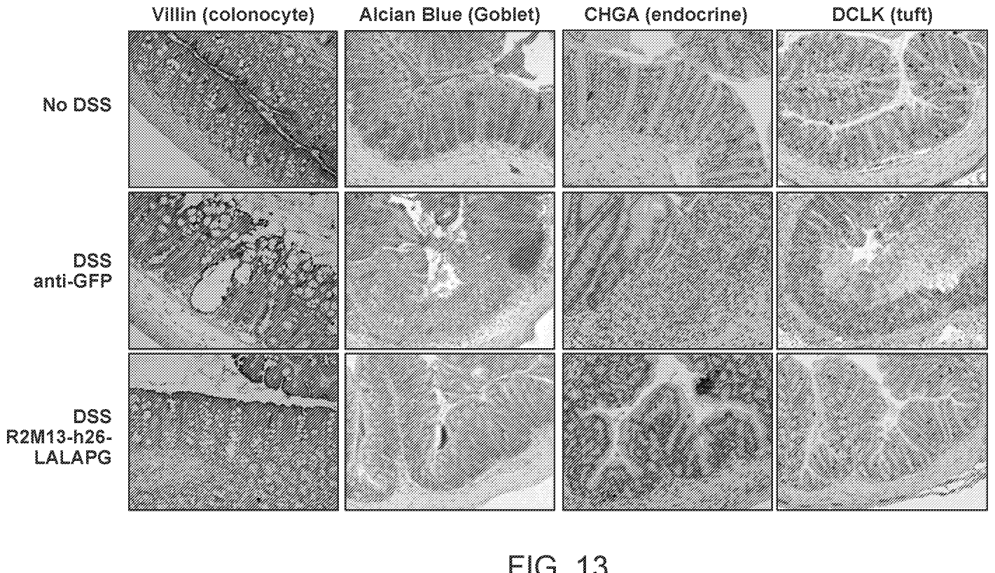

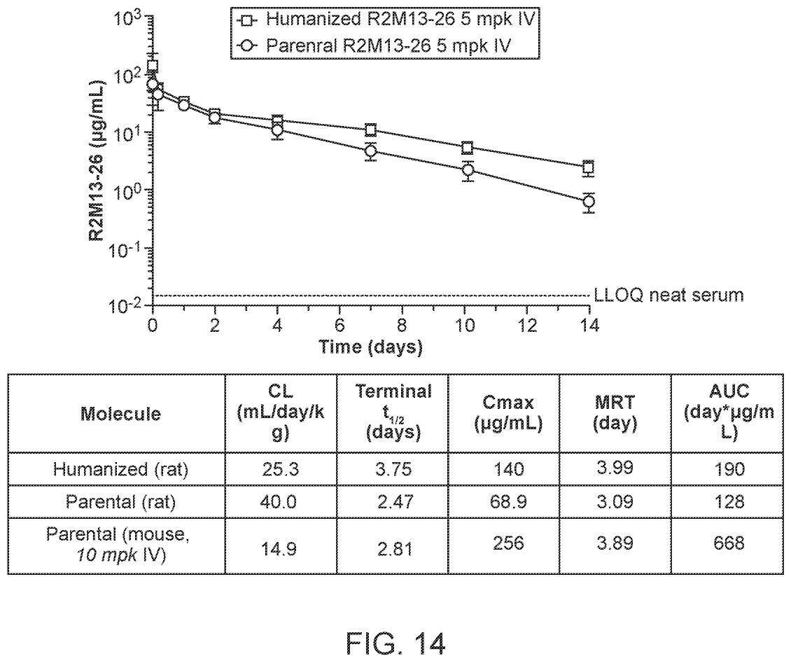

Pharmacokinetics (PK) of the parental R2M13-26 (R2M13-26-LALAPG) and humanized R2M13-26 (R2M13-h26-LALAPG) following intravenous injection was determined by measuring the amount of antibody in serum at various times following administration to rats and compared to data obtained from mice (FIG. 14). Cmax for humanized R2M13-26 (R2M13-h26) was higher than for parental R2M13-26 (R2M13-26), so differences carry over time; however, the fold difference increased over time. Humanized R2M13-26 had lower clearance (25.3 mL/day/kg) than parental R2M13-26 (40.0 mL/day/kg), and humanized R2M13-26 had a longer half-life (3.75 days) than parental R2M13-26 (2.47 days).

### Example 6

### Evaluation of Engineered Wnt Agonists in DSS Chronic Colitis Model

Since R2M13-26 treatment ameliorated acute colitis in DSS model (Example 3), the engineered Wnt agonist, R2M13-26, was tested in a DSS model of chronic colitis at different time points of repeated cycles of DSS-washout, to demonstrate the efficacy of engineered Wnt agonists in the chronic colitis model.

Six-eight-week old C57Bl/6J female mice were obtained from Jackson Laboratories (Bar Harbor, ME, USA) and were housed 4-5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

To induce chronic colitis, female mice were given three cycles of drinking water containing 3.0% (w/v) Dextran Sulfate Sodium (DSS, MP Biomedicals, MFCD00081551) ad libitum for 5 days followed by plain drinking water for 7 days. Groups of mice were either treated with an isotype control antibody (anti-GFP), or treated with 4 doses of R2M13-26-LALAPG (R2M13-26) at 10 mg/kg on days 16, 19, 28 and 31. Animals were terminated on day 33.

R2M13-26 treatment improved body weight and disease activity index in the chronic DSS model. R2M13-26 also improved colon histology. In addition, R2M13-26 reduced the serum inflammatory mediators IL-6 and lipocalin-2 on day 33 at termination of the study (data not shown).

### Example 7

### Effect of Engineered Wnt Agonists on DSS Acute Colitis Model

Examples 3 and 4 demonstrated that the Fzd5,8 specific R2M13-26 and R2M13-h26 Wnt agonists were effective in treating acute mouse colitis (acute DSS) model. The goal of this study was to develop a more comprehensive understanding of the mechanism of action by which R2M13-26 affects cells in the colon throughout the repair process using a similar model system.

Six-seven-week old C57Bl/6J female mice were obtained from Jackson Laboratories (Bar Harbor, ME, USA) and were housed 4-5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

To induce acute colitis, the female mice were given drinking water containing 4.0% (w/v) Dextran Sulfate Sodium (DSS, MP Biomedicals, MFCD00081551) ad libitum for 7 days and drinking water containing 100% (w/v) DSS for 3 days. Groups of mice were either untreated, treated with a control antibody (anti-GFP), or treated with a single i.p. injection of R2M13-26-LALAPG (R2M13-26) on day 4. The 123 mice total were grouped: (Day3=13, day4=13, day5=26, day6=24, day7=26, day10=21), with 91 used for histology endpoints, and 21 for scRNA-seq (group eliminated because of machinery). Daily food intake, BW, fecal score, and occult blood were measured. At termination, the mice were treated as follows: Groups A-E: collect transverse colon for qPCR and histology (terminate groups A on day 3, 4; terminate groups C on day 3, 4, 5, 6, 7; and terminate groups B, D and E on day 5, 6, 7, and 10). Assays/Endpoints included RT-qPCR, histology, scRNA-seq, Fecal score of stool consistency and occult blood, Disease activity index (DAI)=(BW loss+Stool consistency+Blood)/3, Serum inflammatory cytokine (TNF-α, IL-6, lipocalin 2), and Anatomic Pathology: ascending, transverse and descending colon, H&E. Histopathologic scoring criteria included: Inflammation severity, Inflammation extend, Mucosa erosion, crypt proliferation, and Goblet cell loss.

No difference was observed in between PBS and anti-GFP treatment with DSS at day 3 to day 7 (data not shown). However, treatment with R2M13-26 showed healthier colon tissue at 5 days to 10 days, with a noticeable histological improvement by day 7 in R2M13-26 treated animals (data not shown). R2M13-26 improved Fecal Score and BW Loss in DSS Mice (data not shown), thus ameliorating experimental colitis in mice.

RT-qPCR analysis was performed on bulk colon samples to evaluate changes in gene expression. Examination of Wnt induction showed a significant decrease in Axin2 with DSS. R2M13-26 induced expression of Axin2 under no DSS condition. Examination of proliferation markers showed a significant decrease in Ki67 with DSS on Day 4 & rescue by R2M13-26. R2M13-26 rescued Cdk1 downregulation in the presence of DSS. Analysis of stem cell markers showed Lrig1 significantly decreased with DSS on Day 4 and was rescued by R2M13-h26. With respect to clinical markers for IBD, significant upregulation of Gpx2 was seen on Days 5 & 6.

For the scRNA-seq experiments examining gene expression in the DSS model, mice were treated with 4% DSS in their drinking water throughout the duration of the experiment. DSS-treated animals were dosed with 10 mpk R2M13-26 or an anti-GFP antibody on day 4 of the DSS treatment. On day 5 and day 6, three each of Wnt agonist and anti-GFP dosed animals were collected at what was 24-hours and 48-hours post dosing, respectively. Two naïve, uninjured animal samples were also collected at the day 5 and 6 timepoints. Colon, small intestine, spleen and liver tissues were collected at termination and examined or frozen for mRNA analysis. Single cell RNA sequencing (scRNA-seq) was performed on fresh transverse colon samples for single cell isolation, and RT-qPCR was performed on fresh transverse colon for isolation of epithelium only.

Transverse colon was isolated from each animal and feces were removed. After a brief wash in cold PBS, the colon was cut longitudinally to open the tube into a flat sheet, and the tissue was cut into 3-4 mm length fragments. Tissue fragments were incubated in pre-warmed (37° C.) PBS with 5 mM EDTA in a shaker at 37° C. at 150 rpm for 15 minutes. After 15 minutes, the tubes containing the samples were vigorously shaken for 10 seconds to release more epithelial cells. The epithelial cells floating in suspension were removed to a new tube and centrifuged at 200 ref for two minutes to pellet the epithelial cells that dissociated from the tissue. The residual tissue containing the remaining epithelia and lamina propria was then incubated in 8-12.5 mL of lamina propria dissociation buffer at 37° C. for 30 minutes with horizontal shaking at 150 rpm. After pelleting, the epithelial cells were resuspended in 1 mL of TrypLE with DNase1, and the epithelial cells were incubated at 37° C. for about eight minutes and triturated with a P1000 pipette about 25 times. After trituration, the epithelial cells were centrifuged at 500 ref, 4° C., and the supernatant was removed. Epithelial cells were then washed one time in FACS buffer before another round of centrifugation and final resuspension in 0.5 mL of FACS buffer. Following 30 minutes of dissociation in LP dissociation buffer, the remaining tissue fragments and suspension were centrifuged at 500 ref for five minutes. Supernatant was removed down to 1 mL, and the sample was triturated with a P1000 until the solution was homogeneous and all tissue fragments had dissociated. After trituration, the sample was centrifuged at 500 ref for 5 minutes at 4° C. and washed in FACS buffer prior to being resuspended in 1 mL of FACS buffer in preparation for FACS.

All cells were passed through a 40 micron filter prior to FACS. DAPI was used to assess viability by FACS, and only viable (DAPI-negative) cells were collected. Cells were collected from the epithelial fraction and then from the epithelial/lamina propria fraction and combined and counted on a hemocytometer prior to cell capture. Standard 10× Genomics 3′ v3 scRNA-seq protocol was followed, and approximately 4500-5000 cells were loaded per channel. Samples from individual animals were captured per channel. Standard 10× Genomics 3′ v3 scRNA-seq RT, cDNA amplification and sequencing library preparations were followed. Multiplexed sequencing libraries were sequenced on Illumina Nova Seq 6000 S1 lanes.

Illumina read data was processed using the 10× Genomics Cellranger pipeline. Demultiplexed UMI count data was then assessed and low-quality cells and low expression genes were removed. UMI count data was normalized using deconvolution scaling from the R package scran, and cells were clustered using a SNN graph-based clustering approach using the R package scran. Cell type identities were determined using established cell type markers. Differential gene expression was performed at the single cell level for each cluster at using one versus all and pair-wise comparisons within each lineage by using the R package clusterExperiment to run EdgeR. Differential gene expression analysis between experimental conditions was performed with the R package edgeR on pseudobulk samples following aggregation of biological replicate samples based at the lineage level or at the cell type/cluster level. Differential expression comparisons were performed between experimental conditions (DSS-injured verus uninjured and then within the DSS-injury samples for R2M13-26-treated versus anti-GFP treated) along the epithelial lineage and within individual clusters representing cell types within the epithelial lineage for each timepoint (24-hours or 48-hours).

R2M13-26 exerted its effect predominately by directly impacting the epithelial cells of the colon due to the high expression of FZD5 on intestinal epithelial cells and its enrichment in the stem and progenitor cell populations. The following Wnt target genes were increased when one compares the expression of the entire epithelial lineage and all of the cell types that it contains between R2M13-26 and control treatment (Table 7). Molecules were selected if they showed at least a two-fold increase between treatment and control across the epithelial lineage and they had been shown to be direct Wnt targets in the literature. The majority of Wnt target genes were taken from the genetic manipulation and chromatin immunoprecipitation experiments published in Gougelet et al. (2014). Additional scRNA-seq data is shown in Tables 4-6, and 8.

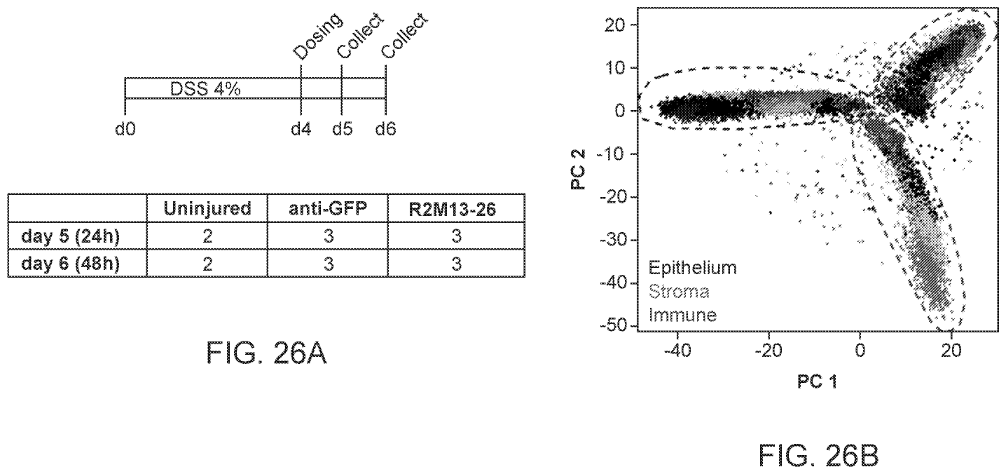

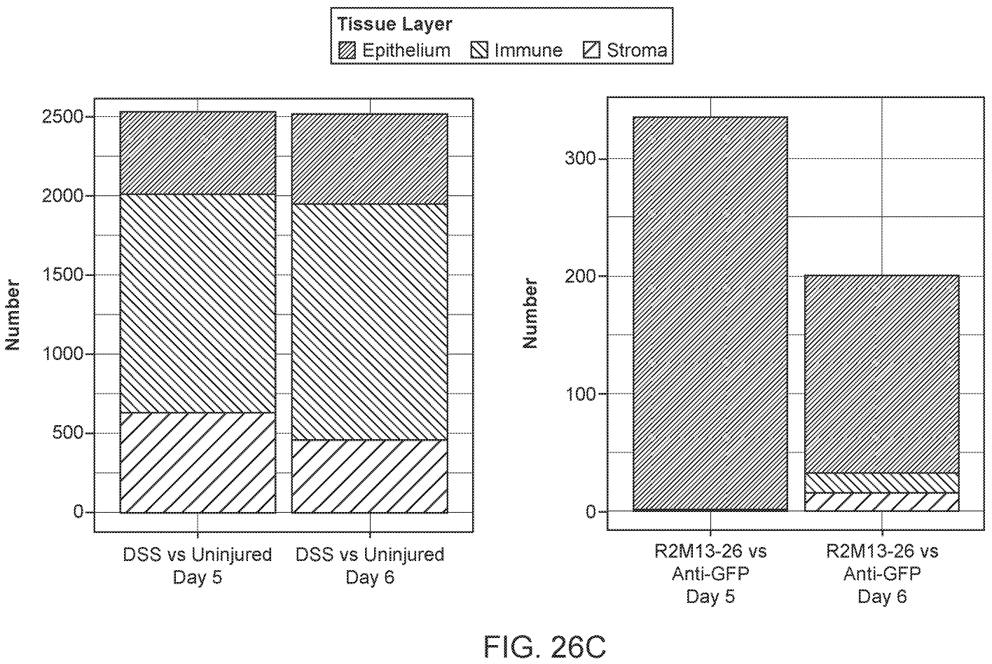

In addition to investigating molecules that demonstrate a significant change across the entire epithelial lineage, the scRNA-seq data was used to examine specific cell types and compare gene expression between R2M13-26 treated cells and control treated cells to identify the Wnt target genes that are increased or decreased in each relevant cell type within the epithelial lineage. This type of differential expression analysis was performed for the following relevant epithelial cell types: stem cells, TA1, TA2, basal goblet cells, injury-induced alternative progenitors (AltEnteroPC), injury-induced alternative enterocytes (AltEntero), enterocyte precursors (EnteroPrecur), goblet cells 1, goblet cells 2, enteroendocrine, and tuft cells. The combined list of Wnt target genes that are modulated in the epithelial lineage as a whole and/or in specific epithelial subtypes with example log 2 fold change is shown in Table 7. Heatmap of epithelial cells detected in the scRNA-seq experiment in shown in FIG. 26B.

A number of molecules were identified as significantly increased or decreased when compared to the expression of the aggregated epithelial lineage and/or any of the cell types that it contains between R2M13-26 and control treatment. Molecules were selected if they showed at least a two-fold change between treatment and control across the epithelial lineage or within at least one epithelial cell type in the acute DSS mouse model of IBD. These molecules are shown in Tables 4-8.

Genes that were increased upon treatment with R2M13-26 were intersected with a list of established cell cycle genes (Giotti et al., 2019) to identify genes involved in cell cycle progression and regulation that were increased by treatment with R2M13-26. The genes identified are listed in Table 4. One of the established roles of Wnt signaling is in the maintenance of stem and progenitor cells, and regulating the cell cycle is an important aspect of that function (Davidson, 2010; Hirata 2013). R2M13-26 promoted the expansion of the stem and progenitor cells in the injured colonic epithelium, which is essential for their ability to regenerate the epithelium. These data indicate that several of these genes are also direct Wnt targets (Table 8).

In addition to promoting expansion of the stem and progenitor cells to facilitate regeneration of the epithelium, Wnt signaling is critical to maintaining and renewing the stem and progenitor cell pool and regulating their differentiation (Pinto et al., 2003; Ma et al., 2016). R2M13-26 promoted repair and regeneration of the epithelium by maintaining the stem and progenitor cells, which was evidenced by increased expression of several key genes involved in this process (Table 8), including Id1 (Hollnagel 1999; Meteoglu 2008; Ruzinova 2003), Nhp2 (Fong 2014; McCann 2020) and Hmga2 (Nishino 2008; Parisi 2020), Foxq1 (Tu 2018; Zhang 2018), and Aldh1 (Tomita 2016). Furthermore, there was also an impact on expression of Areg, a ligand for EGFR signaling, which is important for intestinal stem cell niche maintenance (Fujii 2008; Mahtouk 2005; Suzuki 2010; Takahashi 2020). Yet another interesting molecule that was induced and showed significant increased expression in several stem and progenitor cells following R2M13-26 treatment was glucagon (Gcg). Glucagon can be processed into multiple small peptides, among them are GLP-1 and GLP-2, which play a role in reducing inflammation in IBD. GLP-2 also acts as a growth factor to promote stem and progenitor cell proliferation and regeneration of the epithelial crypts (Drucker 1999; Markovic 2019; Zatorski 2019). These data show that Wnt signaling activation increases expression of glucagon, which would lead to increased levels of GLP-2 and contribute to the expansion of the stem and progenitor cells.

One of the key aspects to tissue repair and epithelial regeneration in addition to regulation of stem and progenitor cell self-renewal and differentiation is the repair of intra and extracellular damage and the re-establishment of the epithelial barrier. To this end, several of the genes induced and/or increased upon treatment of R2M13-26 are associated with these processes (Table 6). For example, Apex1 is critical for DNA repair (Park 2014). Dysfunctions in mucus production and the mucus barrier are key aspects of IBD (Antoni 2014; Dorofeyev 2013; Kim, Ho 2010). Several of the genes increased by treatment with R2M13-26 promote the secretion of mucus and the establishment of the mucus barrier (B3gnt7, Agr2, Muc2, Muc3, Tff3, Fcgbp, and Zgl6). These genes play important roles in mucus production, processing, and secretion of mucus (Agr2: Bergstrom 2014; Park 2009; B3gnt7: Arike 2017; Fcgbp: van der Post 2019; Muc2, Muc3: Arike 2017; Svensson 2018; Kim 2010; Ho 2006; Tff3: Aihara 2017; Zgl6: Bergstrom 2016. Additionally, Sprr2a3, a member of the small proline rich repeat proteins that are involved in epithelial barrier formation (Gibbs 1993) was enriched.

Importantly, reduction or loss of expression of many of these genes are associated with increased severity of colitis in mouse models and/or in development and progression of IBD (Dorofeyev 2013; van der Post 2019). For example, there is a decrease in expression of MUC2, MUC3, and TFF3 in severe CD and UC (Dorofeyev 2012). In mouse colitis models, a reduction in MUC2 makes mice more subsceptible to DSS-induced colitis (Kim, Ho 2010). Furthermore, GWAS studies have identified risk alleles of Agr2 that appear to reduce its expression as promoting IBD (Zheng 2006).

In addition to impacting the epithelial repair and regeneration by regulating stem and progenitor cell proliferation and differentiation, cell repair, and barrier formation, R2M13-26 promoted expression of many genes and pathways associated with reducing the inflammatory response in injury and IBD (Table 5). These molecules have an anti-inflammatory effect and/or their reduction is associated with an increase in inflammation or worsening of IBD.

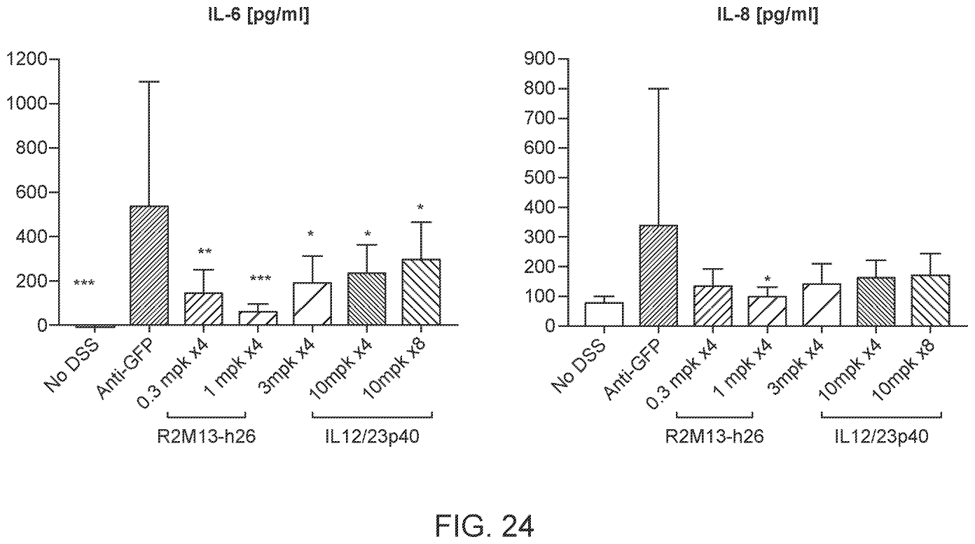

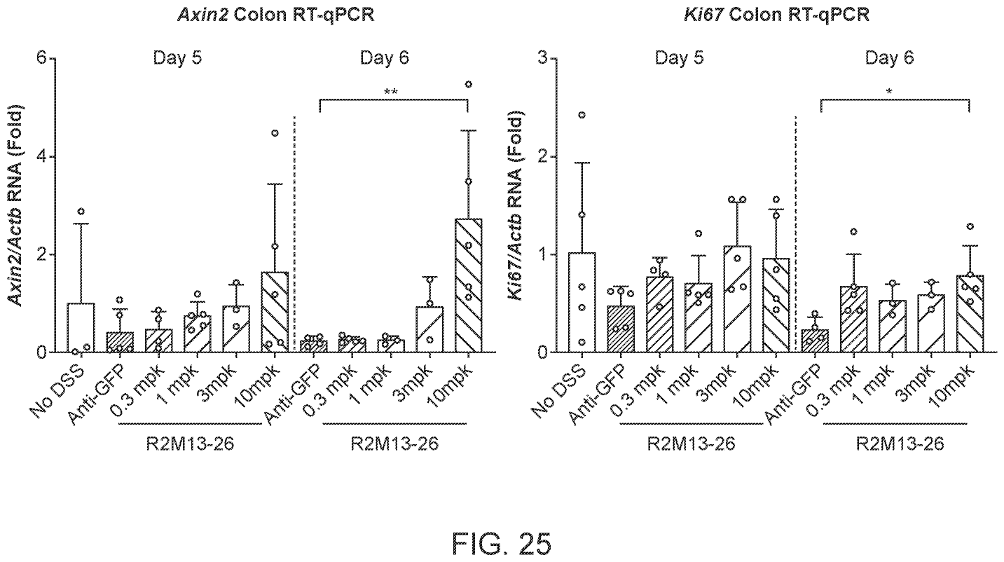

R2M13-26 treated groups showed dose response on serum antibody concentration at 24- and 48-hours post injection, and R2M13-26 exhibited linearity at 1, 3, and 10 mpk dosing. R2M13-26 increased Axin2 and Ki67 expression two days post-single I.P. injection (FIG. 25), and R2M13-26 increased LGR5 expression two days post-injection. R2M13-26 treatment increased Occludin expression at 2 days post-injection.

### Example 8

### Evaluation of Engineered Wnt Agonists in Comparison to Other Agents in DSS Colitis Models

Examples 3 and 4 demonstrated that the Fzd5,8 specific R2M13-26 and R2M13-h26 were effective in treating acute mouse colitis (acute DSS) model, and Example 6 demonstrated that R2M13-26 was effective in treating chronic mouse colitis (chronic DSS) model. The goal of this study was to compare the effectiveness of R2M13-h26 in treating the colitis models to the effectiveness of other agents, including Cyclosporin A, anti-TNF antibodies, and anti-IL-12/23 antibodies. Six to seven-week old C57Bl/6J female mice were obtained from Jackson Laboratories (Bar Harbor, ME, USA) and were housed 4-5 per cage. All animal experimentation was in accordance with the criteria of the “Guide for the Care and Use of Laboratory Animals” prepared by the National Academy of Sciences. Protocols for animal experimentation were approved by the Surrozen Institutional Animal Care and Use Committee.

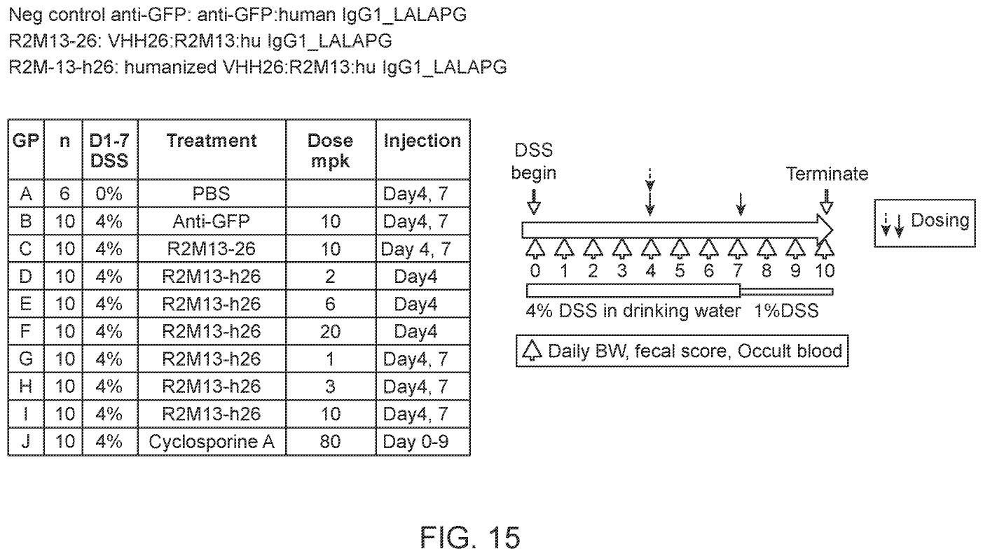

The study with Cyclosporin A is outlined in FIG. 15. To induce acute colitis, the female mice were given drinking water containing 4.0% (w/v) Dextran Sulfate Sodium (DSS, MP Biomedicals, MFCD00081551) ad libitum for 7 days and drinking water containing 1.0% (w/v) DSS for 3 days. Groups of mice were either untreated, treated with an isotype control antibody (anti-GFP), or treated with one I.P. injection of R2M13-h26 at the indicated dose on day 4 or two injections on day 4 and 7, or Cyclosporin A as diagramed.

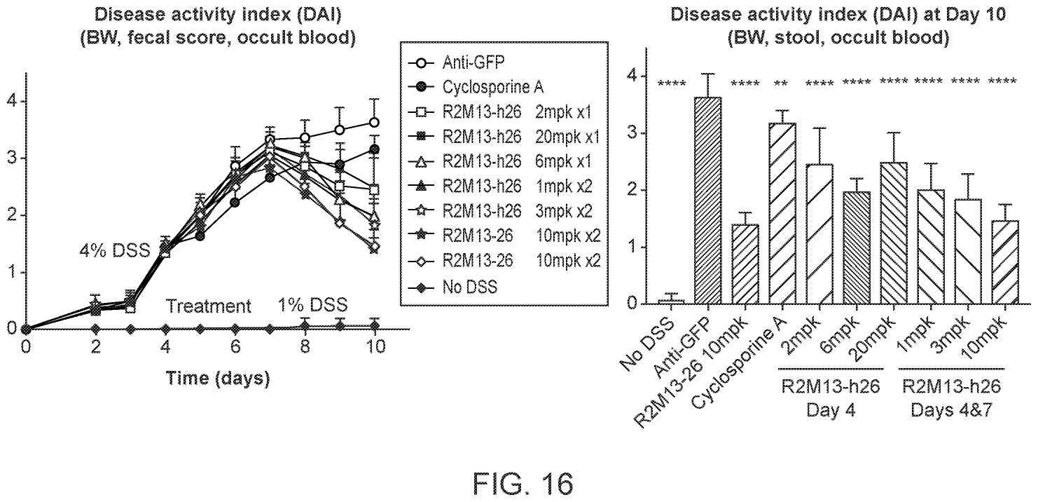

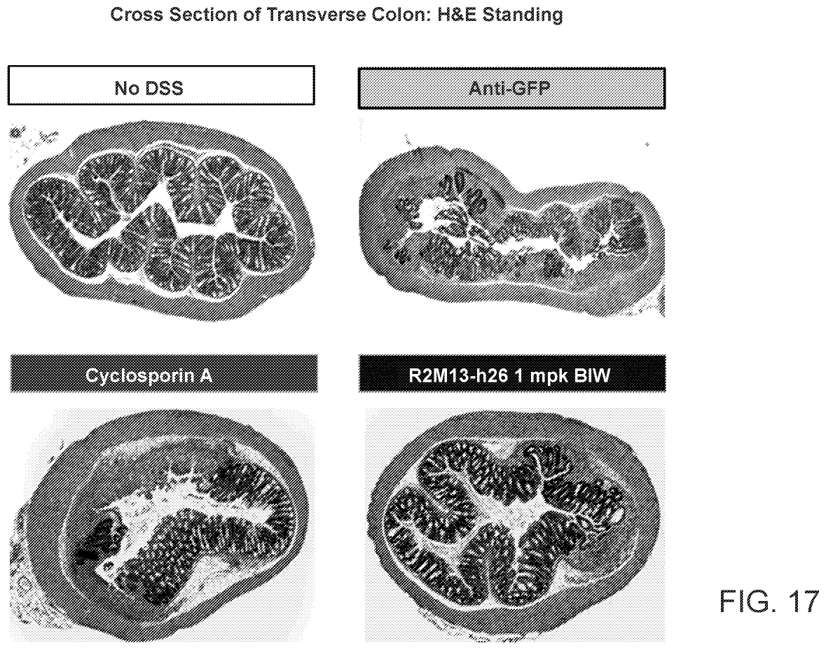

R2M13-h26 treatments improved body weight, decreased fecal score, and decreased Disease Activity Index (DAI) more than Cyclosporine A did (FIG. 16). In addition, R2M13-h26 repaired colon epithelium in vivo more effectively than Cyclosporine A (FIG. 17), improved colon histology score (data not shown), and decreased serum levels of inflammatory cytokines more than Cyclosporin A (data not shown). Overall, R2M13-h26 showed efficacy on repair of colon epithelium, improvement of histology and disease activity index (DAI), and reduction of inflammatory cytokines, at doses as low as 1 mg/kg twice a week or 2 mg/kg single dose. Cyclosporin A showed a mild effect on reducing DAI and lipocalin-2, and was in general much less effective than R2M13-h26.

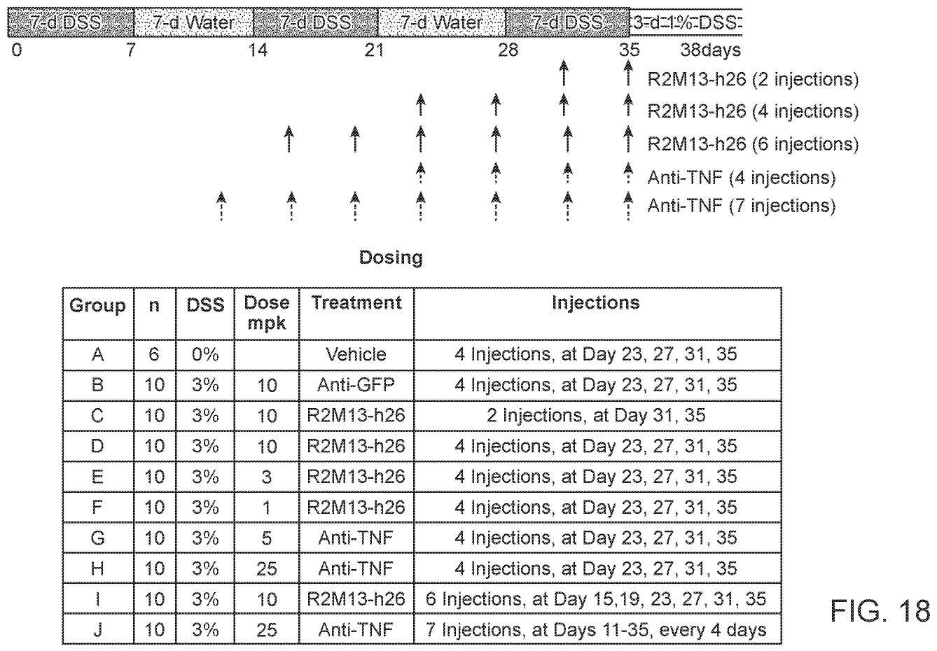

The study comparing R2M13-h26 with anti-TNF in chronic DSS model is outlined in FIG. 18. Mice were administered 3% DSS for three 7-day cycles separated by 7 days off, then a 3-day 1% DSS wash-out period, resulting in chronic intestinal epithelial injury. R2M13-h26 treatment was administered at 1, 3, or 10 mpk for 2, 4, or 6 injections. Anti-TNF was administered at 5 or 25 mpk for 4 or 7 injections. Readout was on day 38.

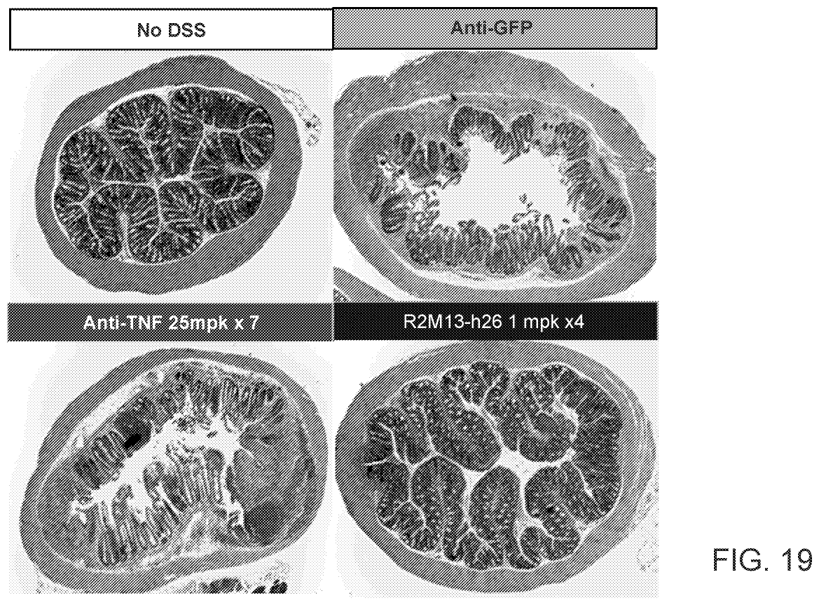

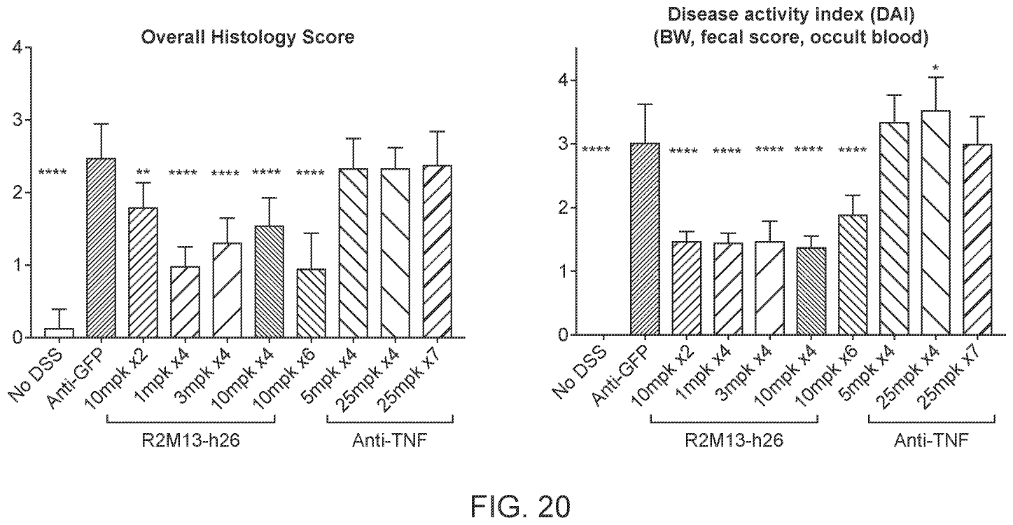

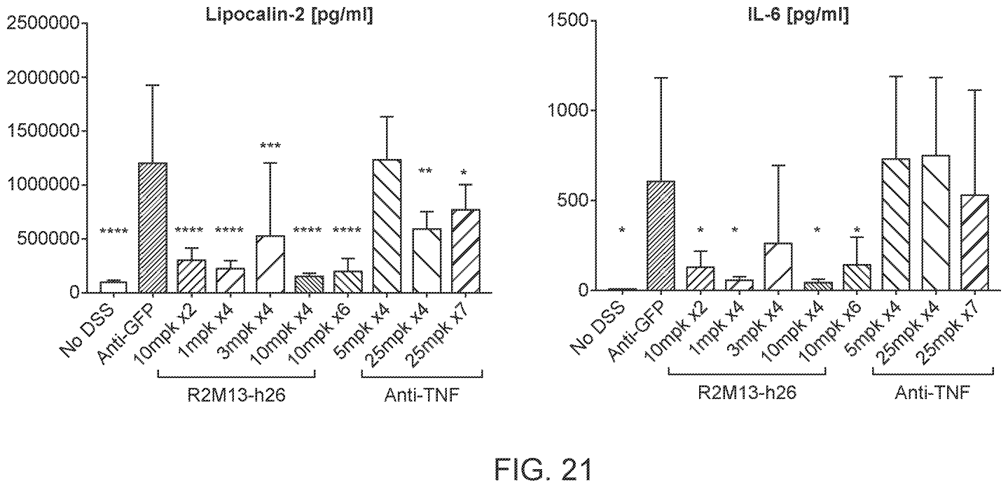

R2M13-h26 repaired colon epithelium more effectively than anti-TNF in the chronic DSS colitis model (FIG. 19). R2M13-h26 decreased colon histology scores, improved body weight, decreased fecal score, and decreased DAI, whereas anti-TNF had no effect on these disease parameters (FIG. 20 and data not shown). R2M13-h26 also reduced serum inflammatory cytokine levels, lipocalin-2 and IL-6, more than anti-TNF in chronic in vivo model (FIG. 21). In a chronic mouse IBD model (chronic DSS with 3 repeated cycles of DSS injuries over 38 days), Fzd5,8 specific R2M13-h26 at various dosing regimen (from 1 mg/kg 4 doses to 10 mg/kg at 2, 4, or 6 doses) was able to reach significant effects on repair of colon epithelium, improvement of histology and disease activity index, and reduction of inflammatory cytokines. In contrast, anti-TNF Ab failed to ameliorate epithelial damage or DAI in the chronic DSS mice.

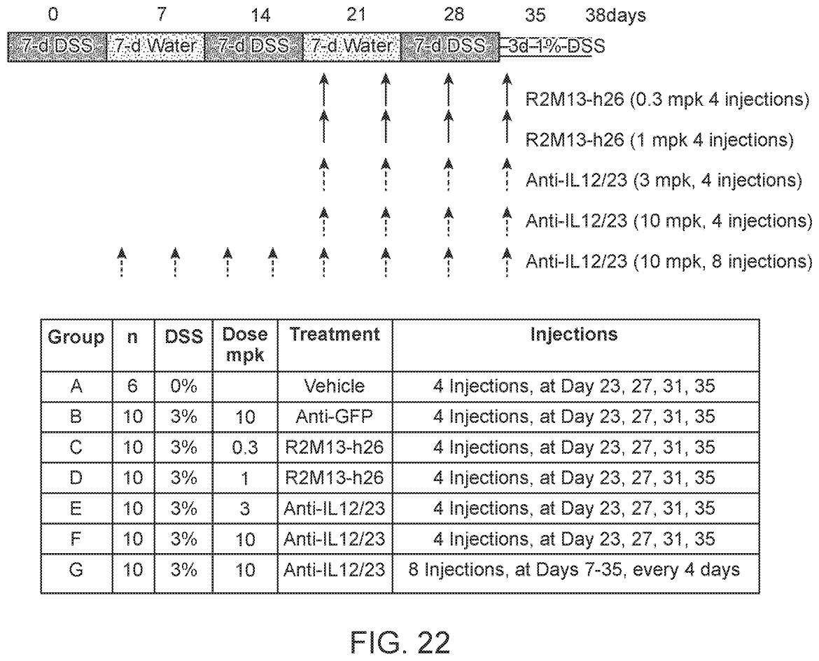

The efficacy of anti-IL12/23p40 relative to R2M13-h26 was also examined in the chronic DSS colitis mouse model with respect to: 1) body weight, fecal score and occult blood, 2) epithelium/barrier repair by histology, and 3) serum inflammatory cytokines. C57BL/6 mice, Female, 6-8 weeks were treated with three cycles of 3.0% Dextran Sulfate Sodium (DSS) to induce chronic colon colitis as outlined in FIG. 22. The first two cycles consisted of seven days with DSS and 7 days on DSS-free water, and the third cycle comprised seven days on 3% DSS and 3 days on 1% DSS. R2M13-h26 treatment was administered at 0.1 and 1 mpk for 4 injections. Anti-IL 12/23 administered at 3 or 10 mpk for 4 or 8 injections. Anti-IL12/23p40 was clone C17.8, from Invivoplus Bioxcell. Readout was conducted on day 38.

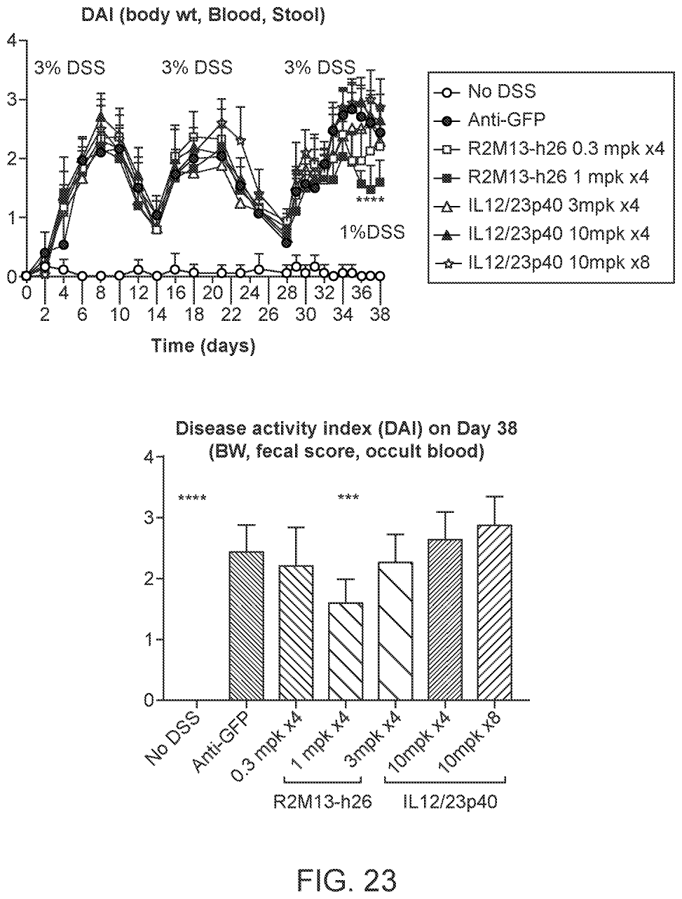

R2M13-h26 treatments decreased Disease Activity Index (DAI) in the chronic DSS mouse model, while anti-IL12/23p40 treatments did not (FIG. 23). In addition, R2M13-h26 treatments decreased serum cytokine levels more effectively than anti-IL12/23 (FIG. 24). This study confirmed that R2M13-h26 was able to repair damaged colon epithelium and decrease serum inflammatory cytokine levels in a chronic DSS mouse model, while the BioXcell's anti-IL12/23p40 monoclonal antibody was not.

### Example 9

### Effect of Engineered Wnt Agonists on Wnt Pathway Activation and Inflammation Reduction

### Selective Wnt Pathway Activation

Fzd5 was shown to be highly expressed in the colon of a mouse model of colitis induced by dextran sodium sulfate, or DSS. In this model, DSS exposure led to disruption of the intestinal barrier resulting in an inflammatory response similar to that seen in IBD patients. R2M13-h26 was observed to bind to DSS-injured intestinal cells, stimulating Wnt signaling as measured by the expression of Axin2, a downstream target gene in the Wnt pathway, restoring tissue architecture, epithelial cell type composition and epithelial barrier function. Mice exposed to DSS for seven days led to the breakdown of the intestinal barrier, which can be readily visualized in stained cross sections of the colon. In the absence of DSS, there was an intact intestinal wall, and the crypts were tightly packed to form a continuous structure. Exposure to DSS followed by treatment with a negative control antibody, anti-GFP, resulted in several effects: a breakdown of the intestinal wall; shrinkage of the colon crypts; and the creation of multiple discontinuous segments by day ten. However, DSS-exposed mice treated with R2M13-h26, administered on days four and seven, led to a dose-dependent repair of this damage, with a dose of 1 mg/kg or higher restoring most of the damage visible by histology. Similar results were observed in a chronic model of DSS. The degree of epithelial repair as measured by histology with R2M13-h26 was greater than what was obtained in additional experiments with Cyclosporine A, an anti-TNF antibody or an anti-IL12/23 antibody.

Histologic staining showed that treatment with R2M13-26 and R2M13-h26 administration led to the restoration of markers of tight junction, the cell-to-cell structures that contribute the intestinal barrier which prevents the free material exchange between the intestinal tract and the abdominal cavity. In healthy intestinal tissue, the zonula occludens 1 protein, or ZO-1, a component of tight junctions, was found as a continuous layer along the intestinal brush boarder. In DSS-damaged intestinal tissue, the continuous expression pattern of ZO-1 was disrupted. Treatment with R2M13-h26-LALAPG restored ZO-1 localization as a continuous layer along the intestinal brush boarder (FIG. 11).

### Inflammation Reduction

In the mouse DSS model, treatment with R2M13-h26 administration led to a significant dose-dependent reduction of a number of inflammatory cytokines such as TNFα, interleukin-6, or IL-6, and interleukin-8, or IL-8. Reductions in cytokine levels were observed both in colon tissue and in serum (data not shown). These results suggest that R2M13-h26 not only has the potential of directly repairing the epithelium but also, as a result, of reducing inflammation.

### Example 10

### Treatment with Wnt Mimetics Rapidly Repaired DSS-Damaged Colon Epithelium

Various Dextran Sodium Sulfite (DSS)-induced colon colitis models were used extensively as preclinical models to study the efficacy of therapeutic compounds and biologics intended to treat ulcerative colitis. An acute severe DSS mouse model was established to study the impact of Wnt signal activation on epithelial repair (see WO 2020/185960A1, incorporated herein by reference in its entirety). In this model, a high percentage of DSS (4%) was used in the first seven days to trigger damage to the colon epithelium. Animals were then maintained on 1% DSS until takedown at day 10 to maintain the established damage and to minimize spontaneous repair of the epithelium. Consistent with previously reported DSS studies (Cooper, H. S., Murthy, S. N., Shah, R. S., & Sedergran, D. J. (1993). Clinicopathologic study of dextran sulfate sodium experimental murine colitis. Laboratory Investigation, 69(2), 238-249), damage to the colon epithelium was visible by hematoxylin and eosin (H&E) stain at day 4 and continued to progress to day 7 (see WO 2020/185960A1). RNAscope in situ hybridization analyses showed a reduction of mRNA expression of Wnt target genes Axin2, Lgr5, Rnf43 as well as Wnt ligands Wnt2b and Wnt5a in the colon epithelium and the surrounding mesenchymal cell layers, respectively. The mRNA expression of the predominant mouse intestinal R-spondin, Rspo3, in the mesenchymal cells underneath the colon crypts was not affected by DSS (see WO 2020/185960A1).

In the established DSS model, mice were injected with two doses of R2M3-26 or a Wnt mimetic targeting FZD1,2,5,7,8 and LRP6 referred to as FA-L6 in Fowler et al. (Fowler, T. W et al., (2021). Development of selective bispecific Wnt mimetics for bone loss and repair. Nature Communications, 12(1). https://doi.org/10.1038/s41467-021-23374-8), starting at day 4 when DSS damage to the epithelium was already visible followed by another dose at day 7, and its effect on epithelial repair was evaluated at day 10, a 6-day treatment. The R2M3-26 treated colon, resembling the colon without DSS treatment, restored crypt architecture with less tissue inflammation as compared to PBS or anti-GFP control treatments (see WO 2020/185960A1). The colon tissues were examined by a pathologist blinded to the treatment and scored for common colitis pathological features (see methods described in Example 1). The histology score consistently showed R2M3-26 effectively repaired the DSS damaged colon tissue, reducing the colitis score from 4.75 to 2.0 (see WO 2020/185960A1).

Since RSPO was previously reported to ameliorate DSS-induced colitis in mice (Zhao et al., 2007), the effect of RSPO2 in the DSS mouse model described herein was also examined. RSPO2 was injected IP either twice weekly or daily starting on day 4 of DSS treatment. While repair to the damaged colon epithelium was observed with RSPO2 treatments, the effect was less significant as compared to the effect of R2M3-26. Similarly, the combined treatment of R2M3-26 and RSPO2, whether twice weekly and daily, restored colon crypt architecture and improved the colon histology but to a lesser extent than R2M3-26 alone (see WO 2020/185960A1).

RSPO alone or combined treatment of RSPO and the Wnt mimetic 18R5-DKK1c was previously shown to stimulate over proliferation of the small intestine stem cells and transient amplifying (TA) cells, leading to growth of small intestine crypt and villi length in normal mice (Yan Kelley S. et al., 2017). In the DSS model described herein, at day 10, an expansion of Ki67 expression by RSPO2 treatments or by a combination treatment of R2M3-26 and RSPO2 in the duodenum and the colon (data not shown) was also observed. However, R2M3-26 alone did not lead to an expansion of Ki67, either in the duodenum or in the colon epithelium at day 10 of the DSS model, consistent with a previous study expressing Wnt agonists in uninjured animals (Yan Kelley S. et al., 2017). The results indicate Wnt agonist treatment alone was able to repair the DSS damaged colon epithelium without causing over proliferation in normal colon or the small intestine.

### Example 11

### R2M13-26, a Fzd5,8-Targeted Wnt Mimetic Stimulated Growth of Mouse Intestinal Organoids

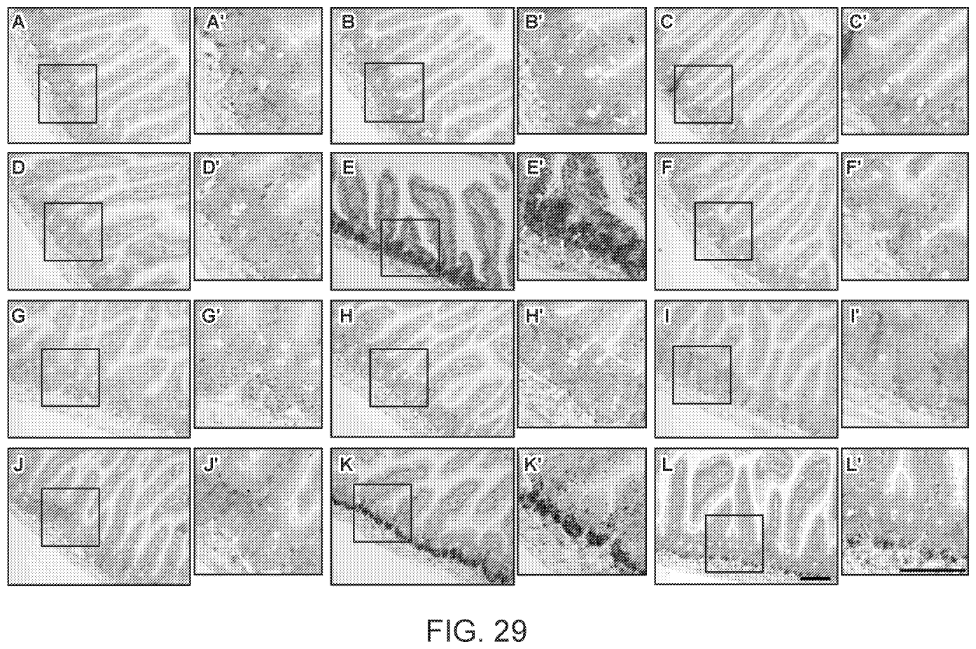

RNAscope in situ hybridization analyses showed that, in the mouse small intestinal epithelium, Fzd5 was expressed at the highest level (FIG. 29E), followed by Fzd1 (FIG. 29A) and Fzd7 (FIG. 29G). Fzd1 and Fzd7 were expressed mostly near the crypt bottom where Lgr5 positive stem cells reside (FIG. 29L). Expression of Fzd5 was concentrated near the crypt-villi border and in the crypt bottom columnar stem cells in the duodenum overlapping with the strong Axin2 positive domain, which was also positive for the stem cell marker Lgr5 (FIG. 29K).

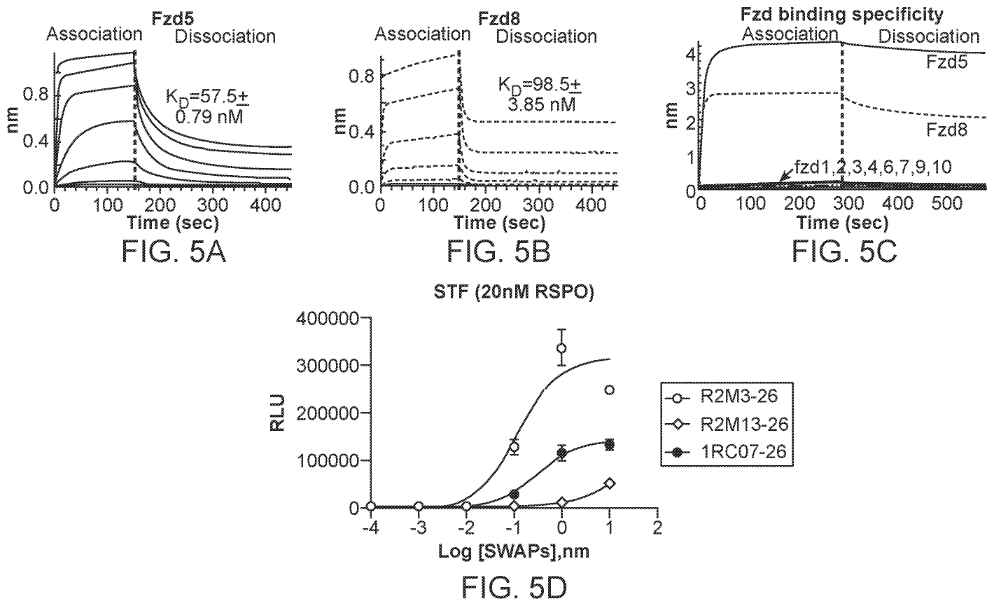

It was then tested whether stimulating Wnt signaling with a Wnt agonist that is specific either to the Fzd5 and Fzd8 subfamily (R2M13-26) or to the Fzd1, 2 and 7 subfamily (1RC07-26) was sufficient to stimulate epithelial cell proliferation in a mouse small intestinal organoid culture. The subfamily specific Wnt mimetics were active in vitro in the Super TopFlash (STF) assay (FIG. 5). Mouse small intestinal organoids were treated with the Porcupine inhibitor IWP2 to inhibit endogenous Wnt ligand secretion in the cultured organoid. When these organoids were subject to no protein treatment or were treated with a control anti-β-gal IgG, the organoids were not maintained and quickly degenerated. In contrast, treatment with R2M3-26, the Fzd1,2,5,7,8 pan specific Wnt mimetic, at a wide dose range was able to stimulate cell proliferation, producing growing transparent sphere-shaped organoids. Both a Fzd5,8-specific Wnt mimetic (“R2M13-26”) and a Fzd1,2,7-specific Wnt mimetic (“1RC07-26”, also referred to as FB-L6 in (Fowler et al., 2021)) were able to stimulate organoid proliferation and growth (see WO 2020/185960A1). The effects of the subfamily specific Wnt mimetics were comparable to the effect of the pan specific agonist.

### Example 12

### The Fzd5,8 Specific Wnt Mimetic, R2M13-26, was Efficacious in Repairing the DSS Damaged Colon Epithelium

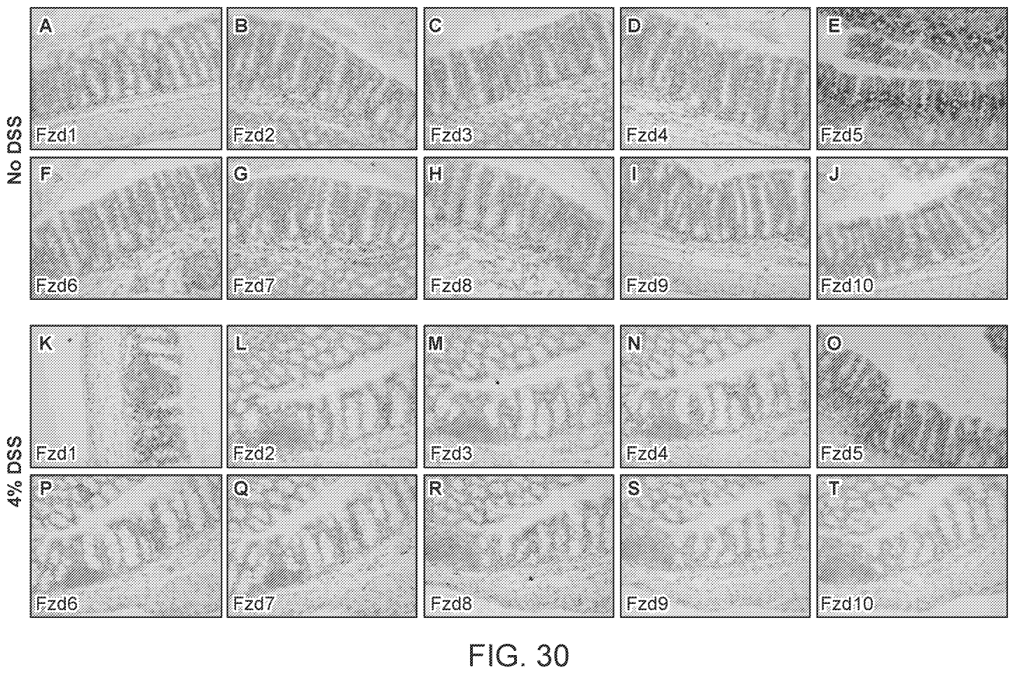

In situ analysis demonstrated that the colon epithelium showed a Fzd expression pattern similar to the small intestine (FIG. 30) and that Fzd5 was also expressed at the highest level among all Fzds in the colon epithelium. This differential expression of Fzds was maintained in the DSS condition albeit that the expression of all Fzds was reduced by DSS (FIG. 30K-30T).

It was next examined if the Fzd subfamily specific mimetics were able to repair the DSS-damaged colon epithelium. In the DSS model, two doses of control anti-GFP IgG treatment or protein treatment were injected via I.P. on day4 and day7, and the animals were sacrificed on day10 for histology and serum analyses. In contrast to the severe tissue damage and inflammation observed in the no protein treatment or the anti-GFP treated colon, both R2M13-26 (Fzd5,8) and 1RC07-26 (Fzd1,2,7) treatment resulted in repair of the colon epithelium. The effect on colon histology from the two Fzd subfamily specific Wnt Mimetics (R2M13-26 and 1RC07-26) was comparable to the Fzd1,2,5,7,8 pan-specific mimetic R2M3-26) (see WO 2020/185960A1).

Similar to R2M3-26 treatment, fecal score and disease activity index (DAI) also improved with R2M13-26 and 1RC07-26 treatment (see WO 2020/185960A1). Improvement in fecal score and DAI was more pronounced with R2M13-26 as compared to R2M3-26 or 1RC07-26. To further understand the extent of tissue repair by the different Wnt mimetics, the colon tissue was again analyzed by a pathologist who was blinded to the treatment groups (see WO 2020/185960A1). Consistent with the DAI, the overall histology score of R2M13-26 treated DSS colon was significantly improved and was better than the 1RC07-26 treated colon, suggesting colitis reduction and epithelial repair from the Fzd5,8-specific Wnt mimetic R2M13-26 was more efficacious than the Fzd1,2,7-specific Wnt mimetic 1RC07-26.

It was then determined whether the colitis reduction observed with the Wnt mimetics would be accompanied by reduced serum cytokine levels. Treatment with each of the three Wnt mimetics reduced the DSS-induced serum levels of the pro-inflammatory cytokines, TNF-α, IL6 and IL-8 (see WO 2020/185960A1).

Efficacy of R2M13-26 in the DSS model was further tested with a dose ranging study where R2M13-26 was injected IP either once on day 4 at 1, 3, 10 and 30 mpk or twice on day 4 and day 7 at 0.3, 1, 3 and 10 mpk. Significant improvement of tissue histology, DAI and histology scores were observed for all dose groups (data not shown). All dose groups also showed significant reduction of serum and tissue levels of pro-inflammatory cytokines TNF-α, IL-6 and IL-8 (see WO 2020/185960A1).

### Example 13

### DSS Injury Caused a Robust Inflammatory Response in all Tissue Layers, but the Predominant, Direct Effect of R2M13-26 was on the Epithelial Cells

scRNA-seq was used to determine what cells first responded to treatment by R2M13-26, how R2M13-26 impacted differentiation of epithelial cells, and whether the effect on reducing inflammatory cytokines occurred directly on immune cells or indirectly through restoration of the epithelium. To study these questions, scRNA-seq was applied to investigate the early transcriptome response of the R2M13-26 treated colon in the acute DSS mouse model. As in the Examples above, 4% (w/v) DSS was administered in the water, and mice were injected IP on day 4 with either 10 mg/kg of the anti-GFP control protein or with 10 mg/kg of R2M13-26 with endpoints at day 5 and day 6, 24- and 48-hours post injection, respectively (FIG. 26A). After filtering, the data set contained 22,717 total cells. Normalization and cluster analysis were applied to the complete data set to identify each lineage/group, subsequently subdivided each lineage/group; dimensionality reduction and cluster analysis were applied on the subset of cells in each (FIG. 26B). There were three major cell groups, immune (4835 cells), mesenchyme/stroma (7509), and the epithelium (10373) (FIG. 26B).

DSS injury had a strong impact on all three lineages at each timepoint, resulting in differential gene expression of between 500 and over 1400 genes in each tissue layer, with the immune lineage displaying the largest number of changes (FIG. 26C).

To understand the impact of R2M13-26 in the DSS model, the effect of DSS injury was first assessed by comparing the DSS, anti-GFP condition to the uninjured condition. DSS induced distinct cell types in each tissue layer or lineage, and this was responsible for a large portion of the lineage level differential gene expression. In the immune lineage, no cell types disappeared upon injury. Rather, by day 5 of DSS treatment, several cell types appeared in the damaged colon samples including activated neutrophils (ActNeutropil), two populations of pro-inflammatory monocytes (InjuryMono1,2), stimulated dendritic cells (ActDendritic), and two groups of B-cells (Beell1_IgM, Bcell2_IgM) enriched for IgM heavy chain gene expression and Ighd. In the stromal cells, DSS injury resulted in the appearance of new populations of fibroblasts expressing inflammatory cytokines and chemokines, consistent with recent reports of pro-inflammatory fibroblasts in UC patients and the DSS mouse model (Kinchen et al., 2018; Smillie et al., 2019). Two groups of fibroblasts consisted almost entirely of injured cells (InjuryCryptFB1, InjuryCryptFB2) (data not shown).

### R2M13-26 Promoted Wnt Target and Cell Cycle Gene Expression and Expanded the Progenitor Cell Populations in the Epithelium Immediately Following Dosing.

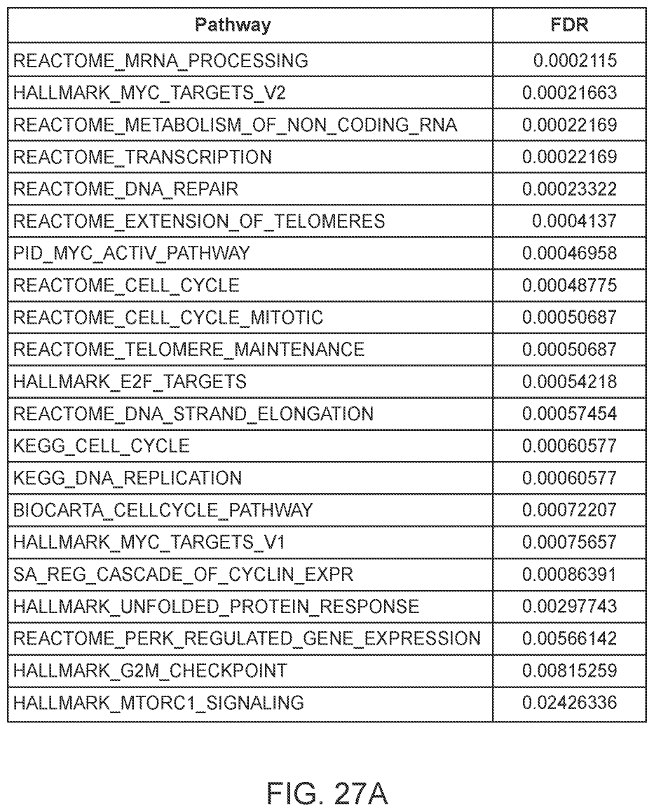

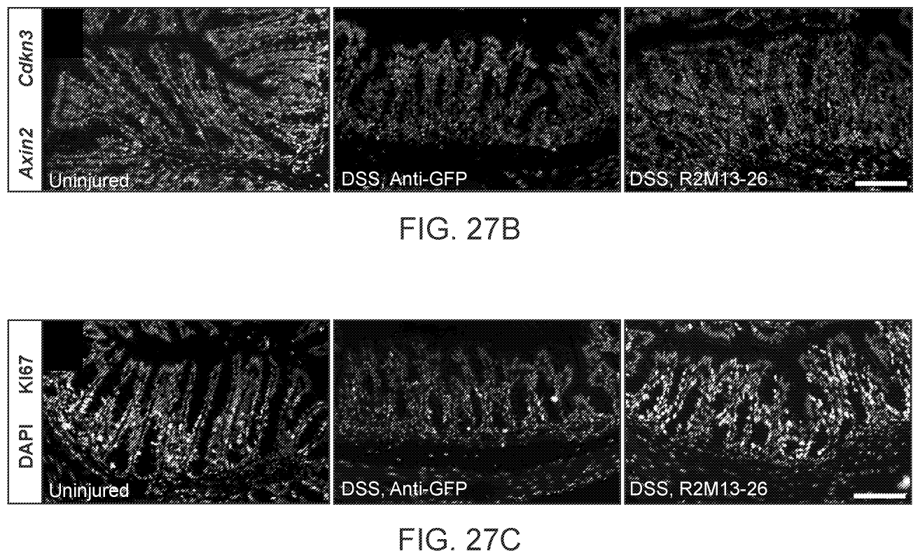

The direct effect of R2M13-26 was predominately on the epithelium. At a global level, at 24-hours after dosing, R2M13-26 led to the differential increase in expression of over 300 genes in the epithelium, but almost no or no genes in the immune and stromal cells/lineages (FIG. 27). R2M13-26 increased expression of a wide range of Wnt target and cell cycle genes in the epithelium, both by expanding expression levels and by expanding the percentage of cells expressing the genes (FIG. 27C; Tables 4 and 7). Table 4 shows the cell cycle genes that were differentially expressed within the epithelial lineage when R2M13-26 treatment was compared to the anti-GFP treatment at either 24-hours or 48-hours. Differential expression was filtered on adjusted p-value (false discovery rate (FDR)) of <0.05.

GSEA on the epithelium comparing R2M13-26 to anti-GFP treatment showed that the cell cycle, telomere maintenance, MTORC signaling, and the UPR stress response were strongly upregulated in the epithelium by R2M13-26 (FIG. 27A).

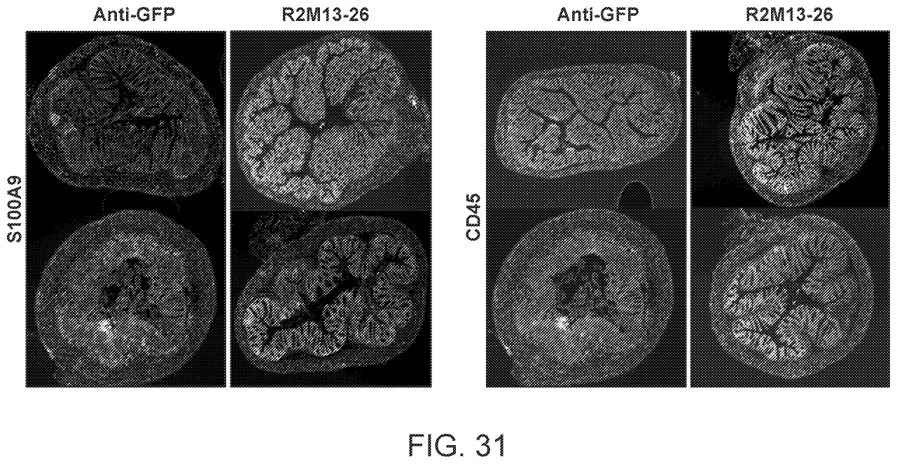

Importantly, Axin2 enrichment at either the lineage or cell type level in any stromal or immune cells was not detected (data not shown). Furthermore, there were very few if any pathways enriched in the stromal or immune cells by GSEA when one compared R2M13-26 to anti-GFP treatment (data not shown), again confirming that the predominant, direct impact of R2M13-26 was on the epithelium at 24-hours after dosing. Here, it is important to highlight that although a major impact of R2M13-26 on the stromal or immune cells early at day 5 or day 6 was not observed, there was a reduction in immune cells and cytokine levels over time that was detectable by day 10 (data not shown), indicating that these changes were secondary to the early, direct impact of R2M13-26 on the epithelium. As shown in FIG. 31, markers of neutrophil infiltration and inflammation both decreased in expression after R2M13-26 treatment.

The predominant cell types impacted by R2M13-26 were the progenitor and precursor populations, including the injury-induced, altered enterocyte cell types. Differential expression analysis revealed a significant increase of Axin2, Rnf43, Cdkn3, and/or other Wnt target genes in several distinct cell types (e.g., AlEnteroPC, TA2, EnteroPrecur). Furthermore, R2M13-26 significantly increased expression of many genes involved in the cell cycle (Table 4) in multiple progenitor cell subtypes in the epithelium, especially the TA2 and injury-specific progenitors (AltEnteroPC). Some of these genes were themselves Wnt targets (e.g., Ccnb1, Cdca3, Aurka, Cdkn3). The increase in Wnt target gene expression was validated, and an expansion of Axin2 and Cdkn3 expression in the colon crypts of the R2M13-26 treated samples was detected (FIG. 27B). Furthermore, the TA1 and TA2 progenitor cells had the highest expression of cell cycle associated genes, and there was an expanded contribution of the R2M13-26 treated samples in these groups at 24-hours after treatment (data not shown), which was consistent with expansion of the progenitors early after dosing.

To validate that the early increase in cell cycle gene expression reflected an increase in the number of proliferative cells, immunohistochemistry analysis was applied using the proliferative cell marker, Ki-67. A robust increase in the number of proliferative cells in the colonic epithelium upon R2M13-26 treatment when compared to the anti-GFP treatment group by 48 hours after dosing was observed (FIG. 27C), consistent with both the scRNA-seq analysis and the increase in cell cycle gene expression detected by RT-qPCR on colon samples. Note that the proliferative cells were not restricted to the base of the crypt but were often positioned near the apical surface.

In addition to increasing expression of genes directly involved in the cell cycle, R2M13-26 also increased expression of several stem/progenitor cell genes such as Lrig1 (Powell et al., 2012), Hmga2 (Nishino, Kim, Chada, & Morrison, 2008; Parisi, Piscitelli, Passaro, & Russo, 2020), and Nhp2, a member of the Dyskerin complex associated with telomere maintenance that was shown to be important for stem cell maintenance (Fong, Ho, Inouye, & Tjian, 2014; McCann, Kavari, Burkholder, Phillips, & Hall, 2020).

In summary, at 24-hours after dosing, R2M13-26 increased Wnt target and cell cycle gene expression in multiple cell types, predominantly in the different subtypes of stem and progenitor cells including the injury-induced, altered enterocyte cell types, leading to an expansion of the progenitor pool.

R2M13-26-Treated Epithelial Cells Differentiated More Quickly after Proliferation.

Time-stamping allowed the determination of where the day 6 (48-hour) cells were enriched relative to the day 5 (24-hour) cells for all three treatment conditions, uninjured, injured/anti-GFP and injured/R2M13-26. The day 5 and day 6 uninjured cells were approximately equally represented in all clusters where uninjured cells were present at both timepoints as expected (FIG. 28A, FIG. 28B, FIG. 28C, and FIG. 28D). However, obvious differences existed between the cell types that were preferentially enriched for the R2M13-26- or anti-GFP-treated day 5 and day 6 injured samples. For the anti-GFP samples, there were more cells in the altered enterocyte groups (AltEntero2, 3) and the TAT groups at day 5 relative to the day 6 timepoint, and there were about equal percentages of cells in the alternative progenitor cells (AltEnteroPC) at both timepoints. In the R2M13-26 samples, there were more TA1 and TA2 cells at day 5 relative to day 6, and a higher percentage of stem cells at day 6 relative to day 5. Importantly, based on real-time-stamping, there was a substantial enrichment of R2M13-26-treated cells in the enterocyte precursors at day 6 and fewer alternative enterocytes expressing high levels of inflammatory genes (AltEntero) relative to the anti-GFP treated samples. Therefore, the day 6 (48-hour) R2M13-26 samples appeared accelerated in differentiating toward enterocytes.

To complement the time-stamp-based observations, a lineage trajectory inference tool, slingshot, was employed. Because there was evidence that some enterocytes were de-differentiating upon DSS injury, the apparent de-differentiating/altered state enterocyte clusters were removed and slingshot was applied to the cell clusters that included at least 5% of cells from the uninjured condition. The combined stem cell/TA2 cells were set as the starting point (FIG. 28A), and slingshot predicted that from the initial starting group, cells would progress toward TAT, Goblet, Tufted, and enteroendocrine in one direction and toward the enterocytes in the other (FIG. 28D). Based on the predicted lineage trajectory pseudotime values, there was a higher percentage of R2M13-26-treated samples that were further along in the enterocyte lineage trajectory by day 6 (48-hours) relative to the control treated cells (FIG. 28E). Further, as shown in FIG. 28E, the progression toward the enterocyte lineage was increased with R2M13-26 treatment. This prediction for the enterocyte lineage was congruent with the actual time-stamping data that the day 6 (48-hour) cells treated with R2M13-26 were accelerated—yet still very early—in the differentiation process toward immature enterocytes.

A reliable standard for validating improved differentiation was that expression of mature, differentiated cell type markers looked more like that of naïve, uninjured colon in the R2M13-26 treatment group relative to the anti-GFP controls on day 10 after DSS-induced damage (6-days after R2M13-26 treatment) (FIG. 13). Unlike the anti-GFP treated control samples, R2M13-26-treated samples had recovered enterocytes, goblet cells, enteroendocrine, and tuft cells.

### R2M13-26 Treatment LED to Epithelial Barrier Restoration and Reduced Inflammation

In the studies looking at day 10 after injury, it was observed that R2M13-26 treatment led to repair of the epithelium at a histological level. At 24-hours after dosing, there was an increase in mucin and barrier associated gene expression in the R2M13-26-treated samples relative to anti-GFP in the TA1 cells. When the expression of the tight junction marker, TJP1 (ZO1), was assessed at day 10, it was observed that its expression was increased and more organized in R2M13-26 versus control-treated colon at day 10, consistent with re-establishment of tight junctions.

In addition to its direct impact on epithelial cell regeneration, R2M13-26 also caused a strong increase in expression of genes involved in glutathione (an antioxidant that may play a role in reducing inflammation) conjugation: two glutathione transfersases (Gstm1, Gstm3) and the glutathione peroxidase, Gpx2, all three of which have been reported to be Wnt target genes (Gougelet et al., 2014; Kipp, Banning, & Brigelius-Flohé, 2007).

### Example 14

### Toxicity Study of R2M13-h26 in Nonhuman Primates (NHP)

To evaluate the toxicity of R2M13-h26 and to evaluate the potential reversibility of any findings following a 4-week recovery period, a 4-week non-GLP (Good Laboratory Practices) toxicity study of R2M13-h26 following intravenous (IV) bolus injection in Cynomolgus monkeys was performed. In addition, the toxicokinetic (TK) characteristics of R2M13-h26 were determined.

Intravenous bolus injections were given once daily to Naïve, female, 2-4 year old Cambodian, cynomolgus macaques (2-4 kg) on Days 1, 8, 15, 22, and 29. Vehicle only was used as a control. Clinical pathology (hematology, chemistry, coagulation, urinalysis) was performed pre-dose and on Days 16 and 30. TK sampling was performed at selected time points during doing and to termination; full TK profiles were sampled on Days 1 and 29, and peak/trough on Day 15. Anti-drug antibody (ADA) sampling was performed pre-dose and on Days 15, 29, and 58. Histopathology was performed at termination on Days 30 and 58. Table 11 show the experimental setup of the TK study.

TABLE 11
Dosage used for TK study in NHP

| | | Dose | Dose | Dose | Females | |
|---|---|---|---|---|---|---|
| | Test | Level | Concentration | Volume | Terminal | Recovery |
| Group | Material | (mg/kg) | (mg/mL) | (mL/kg) | (Day 30) | (Day 58) |
| 1 | Control | 0 | 0 | 5 | 3 | 2 |
| 2 | R2M13-h26 | 3 | 0.6 | 5 | 3 | — |
| 3 | R2M13-h26 | 10 | 2 | 5 | 3 | — |
| 4 | R2M13-h26 | 30 | 6 | 5 | 3 | 2 |

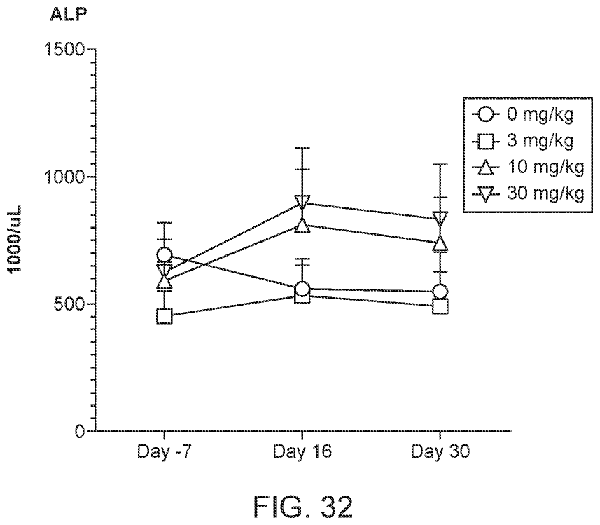

No abnormalities were found in clinical observations, body weight, and food consumption. Modest changes were observed in clinical pathology. Non-adverse, minimal-to-moderate increase in serum alkaline phosphatase (ALP) was observed in the R2M13-h26 groups (FIG. 32), which may be attributed to effects of R2M13-h26 in bone. No gross or microscopic pathology findings were detected. The No Observed Adverse Effect Level (NOAEL) was determined to be 30 mg/kg. No effect on organ weights was detected, and no changes in intestinal segment weights were observed. There was some evidence of increased Axin2 in the duodenum and colon of treated animals (data not shown).

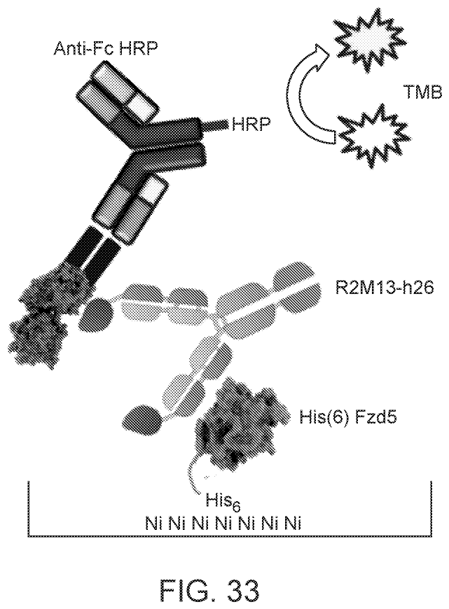

Mean serum concentration of R2M13-h26 was measured using a pharmacokinetic assay which is homogeneous double antigen-based assay, as depicted in FIG. 33. Histidine-conjugated human Frizzled 5 (Fzd5) and mouse low-density lipoprotein receptor-related protein 6 mouse-Fc chimera (Lrp6) were preincubated with R2M13-h26 to form a complex. The Fzd5/R2M13-h26/Lrp6 complex was then applied to a nickel coated plate allowing capture by the Fzd5 histidine tag. Matrix interferences and excess reagents were removed by salt/detergent buffer washes, and the captured complex was subsequently detected by employing a secondary peroxidase-conjugated antibody with specificity to the mouse Fc moiety. The color was developed with 3,3′,5,5′-tetramethylbenzidine (TMB) substrate and HRP reaction was quenched with acidification and the samples were analyzed on a SpectraMax® Paradigm® microplate reader.

TABLE 12
Mean (S.D.) TK parameters for R2M13-h26

| | | | AUC₍₀₋₇₎/D | AUC | | | |
|---|---|---|---|---|---|---|---|
| Dose | | AUC₍₀₋₇₎ | (μg-day/mL// | Accum | Cₘₐₓ | Cₘₐₓ/D | Cₘₐₓ |
| (mg/kg) | Day | (μg-day/mL) | mg/kg) | Ratio | (μg/mL) | (μg/mL//mg/kg) | Accum Ratio |
| 3 | 0 | 107 | 35.6 | NA | 60.0 | 20.0 | NA |
| | | (5.91) | (1.97) | | (3.11) | (1.03) | |
| | 28 | NA | NA | 1.18 | 66.2 | 22.1 | 1.11 |
| | | | | (0.69) | (3.26) | (1.09) | (0.102) |
| 10 | 0 | 375 | 37.5 | NA | 230 | 23.0 | NA |
| | | (23.1) | (2.31) | | (19.5) | (1.95) | |
| | 28 | NA | NA | 1.12 | 243 | 24.3 | 1.06 |
| | | | | (0.026) | (11.4) | (1.14) | (0.042) |
| 30 | 0 | 1065 | 35.5 | NA | 656 | 21.9 | NA |
| | | (221) | (7.37) | | (39.4) | (1.31) | |
| | 28 | 796 | 26.5 | 0.754 | 731 | 24.4 | 1.11 |
| | | (290) | (9.68) | (0.227) | (77.0) | (2.57) | (0.055) |

* AUC₍₀₋₇₎ = area under the concentration-time curve from 0 to 7 days after dosing; D = Dose; Cₘₐₓ = maximum observed serum concentration; Accumulation ratio was compared AUC₍₀₋₁₎ for 3, 10 mg/kg and AUC₍₀₋₇₎ for 30 mg/kg.

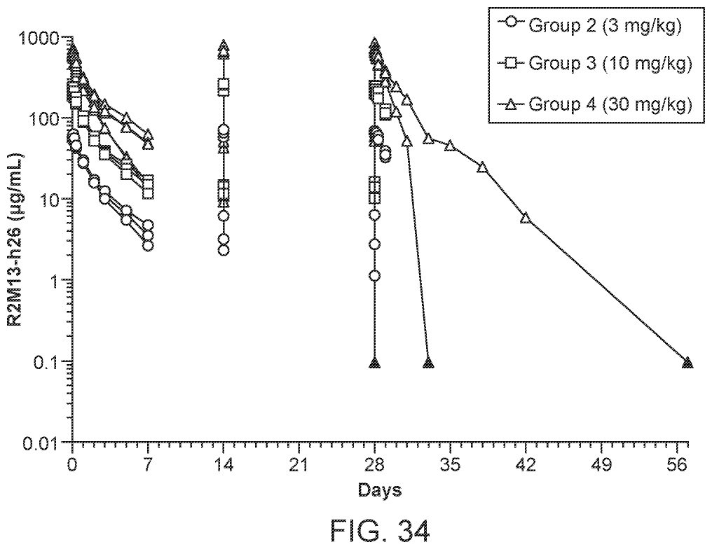

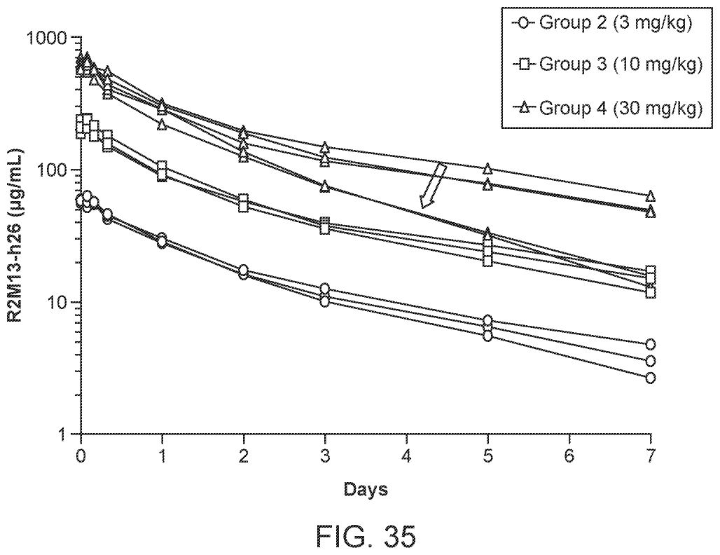

Mean serum concentrations of R2M13-h26 are shown in Table 12 and FIG. 34. The TK was proportional to dosage, and no treatment-related adverse effects were observed. There was no evidence of atypical accumulation or substantive loss of exposure with repeated dosing. One animal was ADA positive in the 30 mg/kg dose group. Additionally, individual serum R2M13-h26 concentrations were measured following the first dose. As shown in FIG. 35, two animals in the 30 mg/kg dose group had accelerated clearance starting 3 days after dosing. These animals also had consistently lower trough concentrations of R2M13-h26 during the study period. One animal was found to have rapid serum clearance at the end of study.

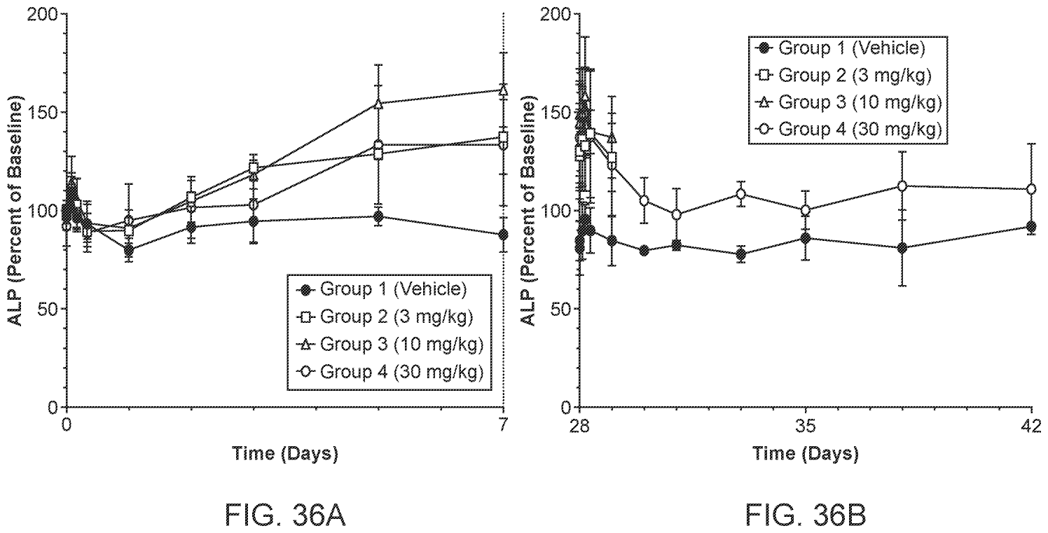

Mild (<2× from baseline), non-adverse, dose-dependent increase in serum ALP was observed with a return to baseline upon cessation of dosing (FIG. 36). Isozyme analysis indicated that the increased ALP may be of bone origin.

Overall, results indicated that R2M13-h26 was well-tolerated in NHP at up to 30 mg/kg/week for four weeks. No treatment-related adverse effects were observed in any parameter. The exposure was consistent with expectations that indicate a successful study, with some evidence indicating reduced exposure in a small fraction of animals. The increase in ALP provided evidence of PD effect with possible saturation.

### Example 15

### Pharmacokinetics (PK) Study of R2M13-h26-LALAPG in Nonhuman Primates (NHP)

Pharmacokinetics (PK) of R2M13-h26 was evaluated in NHP following a single dose of R2M13-h26 intravenous (IV) bolus injection.

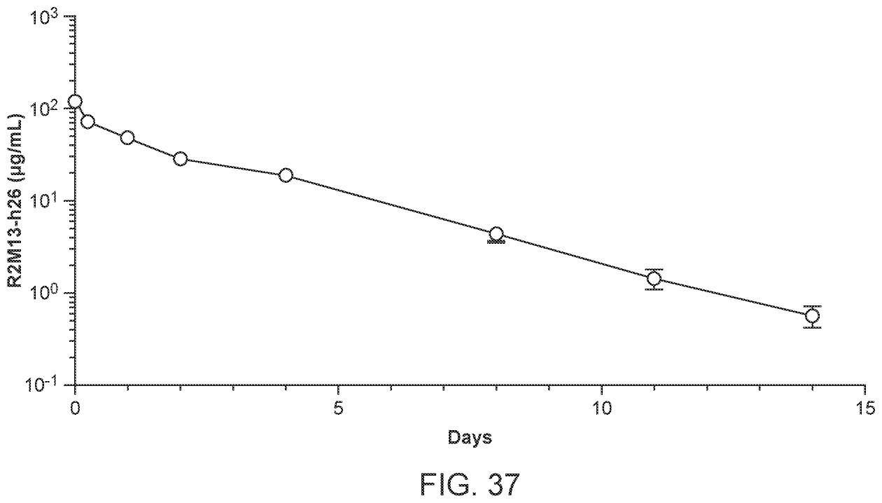

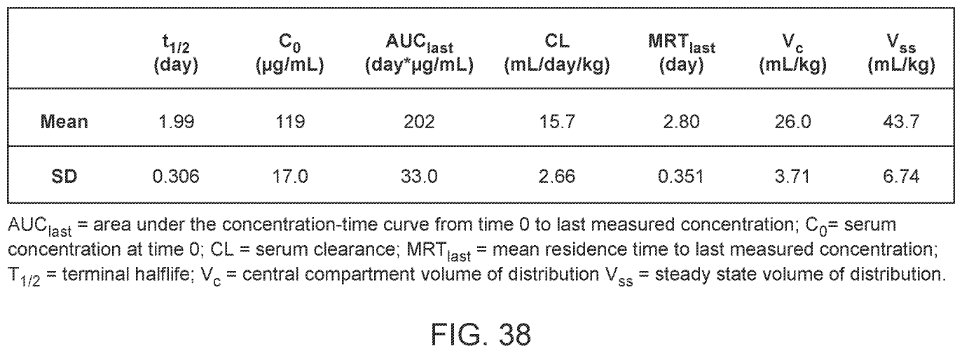

A single IV dose of 3 mg/kg R2M13-h26 was given to each of 4 female cynomolgus monkeys on Day 0. Serum samples was collected at elected time points until 21 days after dosing. Mean serum R2M13-h26 concentrations were measured using the pharmacokinetic assay described in Example 14 and results are shown in FIG. 37. PK parameters for R2M13-h26 including t_{1/2}, AUCₗₐₛₜ, C₀, serum clearance, MRTₗₐₛₜ, V_{c}, and Vₛₛ were determined and presented in FIG. 38.

Results indicated that the PK of R2M13-h26 was consistent with IgG levels and showed low volume of distribution. Clearance of R2M13-h26 was slightly faster than typical IgG in NHP. As such, these results suggest R2M13-h26 can be safely administered to NHPs, with PK suitable for use in humans.

TABLE 4
Illustrative cell cycle genes modulated in response to Wnt agonist
(logFC = Log2 Fold Change; FDR = False Discovery Rate)

| Gene | Full Gene Name | Cell Type | Condition | logFC | FDR |
|---|---|---|---|---|---|
| Tipin | TIMELESS interacting | epithelium | R2M13-26 minus anti- | 1.632145 | 0.00143981 |
| | protein | | GFP d 5_24 h | | |
| Pa2g4 | proliferation-associated 2G4 | epithelium | R2M13-26 minus anti- | 1.239787 | 0.002379216 |
| | | | GFP d 5_24 h | | |
| Rfc4 | replication factor C subunit 4 | epithelium | R2M13-26 minus anti- | 1.476028 | 0.003082582 |
| | | | GFP d 5_24 h | | |
| Fen1 | flap structure-specific | epithelium | R2M13-26 minus anti- | 1.528652 | 0.003082582 |
| | endonuclease 1 | | GFP d 5_24 h | | |
| Spc24 | SPC24 component of NDC80 | epithelium | R2M13-26 minus anti- | 1.751851 | 0.0040443 |
| | kinetochore complex | | GFP d 5_24 h | | |
| Mthfd1 | methylenetetrahydrofolate | epithelium | R2M13-26 minus anti- | 1.050683 | 0.004094943 |
| | dehydrogenase, | | GFP d 5_24 h | | |
| | cyclohydrolase and | | | | |
| | formyltetrahydrofolate | | | | |
| | synthetase 1 | | | | |
| Dtymk | deoxythymidylate kinase | epithelium | R2M13-26 minus anti- | 1.319476 | 0.004146102 |
| | | | GFP d 5_24 h | | |
| Ran | RAN, member RAS | epithelium | R2M13-26 minus anti- | 1.379784 | 0.004442951 |
| | oncogene family | | GFP d 5_24 h | | |
| Mcm5 | minichromosome | epithelium | R2M13-26 minus anti- | 1.26413 | 0.0050001 |
| | maintenance complex | | GFP d 5_24 h | | |
| | component 5 | | | | |
| Cdca8 | cell division cycle associated | epithelium | R2M13-26 minus anti- | 1.40295 | 0.0050001 |
| | 8 | | GFP d 5_24 h | | |
| Chaf1b | chromatin assembly factor 1 | epithelium | R2M13-26 minus anti- | 1.562627 | 0.0050001 |
| | subunit B | | GFP d 5_24 h | | |
| Tyms | thymidylate synthetase | epithelium | R2M13-26 minus anti- | 1.183866 | 0.0050001 |
| | | | GFP d 5_24 h | | |
| Birc5 | baculoviral IAP repeat | epithelium | R2M13-26 minus anti- | 1.725848 | 0.0050001 |
| | containing 5 | | GFP d 5_24 h | | |
| Rfc5 | replication factor C subunit 5 | epithelium | R2M13-26 minus anti- | 1.34582 | 0.005698527 |
| | | | GFP d 5_24 h | | |
| Cdk1 | cyclin dependent kinase 1 | epithelium | R2M13-26 minus anti- | 1.538018 | 0.005842661 |
| | | | GFP d 5_24 h | | |
| Prim1 | DNA primase subunit 1 | epithelium | R2M13-26 minus anti- | 1.271932 | 0.005959274 |
| | | | GFP d 5_24 h | | |
| Mcm6 | minichromosome | epithelium | R2M13-26 minus anti- | 1.23061 | 0.005959274 |
| | maintenance complex | | GFP d 5_24 h | | |
| | component 6 | | | | |
| Stmn1 | stathmin 1 | epithelium | R2M13-26 minus anti- | 1.427388 | 0.005959274 |
| | | | GFP d 5_24 h | | |
| Pclaf | PCNA clamp associated | epithelium | R2M13-26 minus anti- | 1.480806 | 0.005959274 |
| | factor | | GFP d 5_24 h | | |
| Nup85 | nucleoporin 85 | epithelium | R2M13-26 minus anti- | 1.052163 | 0.005959274 |
| | | | GFP d 5_24 h | | |
| Ube2t | ubiquitin conjugating enzyme | epithelium | R2M13-26 minus anti- | 1.495746 | 0.006507412 |
| | E2 T | | GFP d 5_24 h | | |
| Pbk | PDZ binding kinase | epithelium | R2M13-26 minus anti- | 1.568091 | 0.006639875 |
| | | | GFP d 5_24 h | | |
| Nup43 | nucleoporin 43 | epithelium | R2M13-26 minus anti- | 1.663265 | 0.006639875 |
| | | | GFP d 5_24 h | | |
| Hat1 | histone acetyltransferase 1 | epithelium | R2M13-26 minus anti- | 1.339853 | 0.006639875 |
| | | | GFP d 5_24 h | | |
| Lig1 | DNA ligase 1 | epithelium | R2M13-26 minus anti- | 1.011951 | 0.006639875 |
| | | | GFP d 5_24 h | | |
| Mcm7 | minichromosome | epithelium | R2M13-26 minus anti- | 1.294281 | 0.006639875 |
| | maintenance complex | | GFP d 5_24 h | | |
| | component 7 | | | | |
| Ruvb12 | RuvB like AAA ATPase 2 | epithelium | R2M13-26 minus anti- | 1.255863 | 0.007136635 |
| | | | GFP d 5_24 h | | |
| Cenph | centromere protein H | epithelium | R2M13-26 minus anti- | 1.678124 | 0.007184767 |
| | | | GFP d 5_24 h | | |
| Pold2 | DNA polymerase delta 2, | epithelium | R2M13-26 minus anti- | 1.174795 | 0.007573386 |
| | accessory subunit | | GFP d 5_24 h | | |
| Cks1b | CDC28 protein kinase | epithelium | R2M13-26 minus anti- | 1.310798 | 0.008380365 |
| | regulatory subunit 1B | | GFP d 5_24 h | | |
| Dhfr | dihydrofolate reductase | epithelium | R2M13-26 minus anti- | 1.211103 | 0.008576568 |
| | | | GFP d 5_24 h | | |
| Gmnn | geminin DNA replication | epithelium | R2M13-26 minus anti- | 1.054867 | 0.008586835 |
| | inhibitor | | GFP d 5_24 h | | |
| Uhrf1 | ubiquitin like with PHD and | epithelium | R2M13-26 minus anti- | 1.252776 | 0.008602422 |
| | ring finger domains 1 | | GFP d 5_24 h | | |
| Mcm2 | minichromosome | epithelium | R2M13-26 minus anti- | 1.219725 | 0.008619493 |
| | maintenance complex | | GFP d 5_24 h | | |
| | component 2 | | | | |
| Pole3 | DNA polymerase epsilon 3, | epithelium | R2M13-26 minus anti- | 1.089279 | 0.008851502 |
| | accessory subunit | | GFP d 5_24 h | | |
| Cenpm | centromere protein M | epithelium | R2M13-26 minus anti- | 1.737091 | 0.008918625 |
| | | | GFP d 5_24 h | | |
| Aurka | aurora kinase A | epithelium | R2M13-26 minus anti- | 1.251548 | 0.009399304 |
| | | | GFP d 5_24 h | | |
| Orc6 | origin recognition complex | epithelium | R2M13-26 minus anti- | 1.264605 | 0.009990261 |
| | subunit 6 | | GFP d 5_24 h | | |
| Smc2 | structural maintenance of | epithelium | R2M13-26 minus anti- | 1.191401 | 0.009990261 |
| | chromosomes 2 | | GFP d 5_24 h | | |
| Dut | deoxyuridine triphosphatase | epithelium | R2M13-26 minus anti- | 1.373491 | 0.009990261 |
| | | | GFP d 5_24 h | | |
| Cdkn3 | cyclin dependent kinase | epithelium | R2M13-26 minus anti- | 1.659597 | 0.009990261 |
| | inhibitor 3 | | GFP d 5_24 h | | |
| Rrm2 | ribonucleotide reductase | epithelium | R2M13-26 minus anti- | 1.246787 | 0.010234729 |
| | regulatory subunit M2 | | GFP d 5_24 h | | |
| Cdc20 | cell division cycle 20 | epithelium | R2M13-26 minus anti- | 1.294395 | 0.010451406 |
| | | | GFP d 5_24 h | | |
| Nup37 | nucleoporin 37 | epithelium | R2M13-26 minus anti- | 1.41603 | 0.01046245 |
| | | | GFP d 5_24 h | | |
| Ccne1 | cyclin E1 | epithelium | R2M13-26 minus anti- | 1.73646 | 0.010798551 |
| | | | GFP d 5_24 h | | |
| Ccnb2 | cyclin B2 | epithelium | R2M13-26 minus anti- | 1.381396 | 0.011562403 |
| | | | GFP d 5_24 h | | |
| Rrm1 | ribonucleotide reductase | epithelium | R2M13-26 minus anti- | 1.008432 | 0.011604874 |
| | catalytic subunit M1 | | GFP d 5_24 h | | |
| Rfc3 | replication factor C subunit 3 | epithelium | R2M13-26 minus anti- | 1.029058 | 0.011933798 |
| | | | GFP d 5_24 h | | |
| Tk1 | thymidine kinase 1 | epithelium | R2M13-26 minus anti- | 1.262877 | 0.013481779 |
| | | | GFP d 5_24 h | | |
| Cdca7 | cell division cycle associated | epithelium | R2M13-26 minus anti- | 1.171612 | 0.013644871 |
| | 7 | | GFP d 5_24 h | | |
| Haus4 | HAUS augmin like complex | epithelium | R2M13-26 minus anti- | 1.172548 | 0.013787289 |
| | subunit 4 | | GFP d 5_24 h | | |
| Melk | maternal embryonic leucine | epithelium | R2M13-26 minus anti- | 1.43941 | 0.014216405 |
| | zipper kinase | | GFP d 5_24 h | | |
| Mybl2 | MYB proto-oncogene like 2 | epithelium | R2M13-26 minus anti- | 1.346163 | 0.015042194 |
| | | | GFP d 5_24 h | | |
| Incenp | inner centromere protein | epithelium | R2M13-26 minus anti- | 1.02303 | 0.015042194 |
| | | | GFP d 5_24 h | | |
| Asf1b | anti-silencing function 1B | epithelium | R2M13-26 minus anti- | 1.743891 | 0.01529826 |
| | histone chaperone | | GFP d 5_24 h | | |
| Mcm3 | minichromosome | epithelium | R2M13-26 minus anti- | 1.204862 | 0.015584636 |
| | maintenance complex | | GFP d 5_24 h | | |
| | component 3 | | | | |
| Ndc1 | NDC1 transmembrane | epithelium | R2M13-26 minus anti- | 1.014847 | 0.016082636 |
| | nucleoporin | | GFP d 5_24 h | | |
| Cdt1 | chromatin licensing and DNA | epithelium | R2M13-26 minus anti- | 1.018289 | 0.017100865 |
| | replication factor 1 | | GFP d 5_24 h | | |
| Cenpq | centromere protein Q | epithelium | R2M13-26 minus anti- | 1.256335 | 0.018231158 |
| | | | GFP d 5_24 h | | |
| Cenpu | centromere protein U | epithelium | R2M13-26 minus anti- | 1.42333 | 0.018231158 |
| | | | GFP d 5_24 h | | |
| Fbxo5 | F-box protein 5 | epithelium | R2M13-26 minus anti- | 1.247194 | 0.018773023 |
| | | | GFP d 5_24 h | | |
| Ccnb1 | cyclin B1 | epithelium | R2M13-26 minus anti- | 1.15428 | 0.018882603 |
| | | | GFP d 5_24 h | | |
| Rad51 | RAD51 recombinase | epithelium | R2M13-26 minus anti- | 1.254654 | 0.020252318 |
| | | | GFP d 5_24 h | | |
| Chaf1a | chromatin assembly factor 1 | epithelium | R2M13-26 minus anti- | 1.060375 | 0.021679924 |
| | subunit A | | GFP d 5_24 h | | |
| Chtf18 | chromosome transmission | epithelium | R2M13-26 minus anti- | 1.61156 | 0.023550092 |
| | fidelity factor 18 | | GFP d 5_24 h | | |
| Cdc45 | cell division cycle 45 | epithelium | R2M13-26 minus anti- | 1.625426 | 0.023829659 |
| | | | GFP d 5_24 h | | |
| Cenpw | centromere protein W | epithelium | R2M13-26 minus anti- | 1.167276 | 0.024072569 |
| | | | GFP d 5_24 h | | |
| Dscc1 | DNA replication and sister | epithelium | R2M13-26 minus anti- | 1.628844 | 0.024595671 |
| | chromatid cohesion 1 | | GFP d 5_24 h | | |
| Dctpp1 | dCTP pyrophosphatase 1 | epithelium | R2M13-26 minus anti- | 1.252019 | 0.025060476 |
| | | | GFP d 5_24 h | | |
| Orc2 | origin recognition complex | epithelium | R2M13-26 minus anti- | 1.123944 | 0.025075902 |
| | subunit 2 | | GFP d 5_24 h | | |
| Aurkb | aurora kinase B | epithelium | R2M13-26 minus anti- | 1.390542 | 0.025455962 |
| | | | GFP d 5_24 h | | |
| Exo1 | exonuclease 1 | epithelium | R2M13-26 minus anti- | 1.721342 | 0.025512129 |
| | | | GFP d 5_24 h | | |
| Ticrr | TOPBP1 interacting | epithelium | R2M13-26 minus anti- | 1.509485 | 0.02716189 |
| | checkpoint and replication | | GFP d 5_24 h | | |
| | regulator | | | | |
| Cdca3 | cell division cycle associated | epithelium | R2M13-26 minus anti- | 1.190729 | 0.027458596 |
| | 3 | | GFP d 5_24 h | | |
| Slc29a1 | solute carrier family 29 | epithelium | R2M13-26 minus anti- | 1.167626 | 0.02841159 |
| | member 1 (Augustine blood | | GFP d 5_24 h | | |
| | group) | | | | |
| Fignl1 | fidgetin like 1 | epithelium | R2M13-26 minus anti- | 1.279447 | 0.028531028 |
| | | | GFP d 5_24 h | | |
| Cenpa | centromere protein A | epithelium | R2M13-26 minus anti- | 1.022438 | 0.029439811 |
| | | | GFP d 5_24 h | | |
| Cenpp | centromere protein P | epithelium | R2M13-26 minus anti- | 1.372066 | 0.030422111 |
| | | | GFP d 5_24 h | | |
| Atp23 | ATP23 metallopeptidase and | epithelium | R2M13-26 minus anti- | 1.343583 | 0.03094977 |
| | ATP synthase assembly | | GFP d 5_24 h | | |
| | factor homolog | | | | |
| Rad54l | RAD54 like | epithelium | R2M13-26 minus anti- | 1.547449 | 0.03230685 |
| | | | GFP d 5_24 h | | |
| Spc25 | SPC25 component of NDC80 | epithelium | R2M13-26 minus anti- | 1.114321 | 0.032395687 |
| | kinetochore complex | | GFP d 5_24 h | | |
| Clspn | claspin | epithelium | R2M13-26 minus anti- | 1.223129 | 0.032495844 |
| | | | GFP d 5_24 h | | |
| Sgo1 | shugoshin 1 | epithelium | R2M13-26 minus anti- | 1.1496 | 0.032993748 |
| | | | GFP d 5_24 h | | |
| Dtl | denticleless E3 ubiquitin | epithelium | R2M13-26 minus anti- | 1.160475 | 0.032993748 |
| | protein ligase homolog | | GFP d 5_24 h | | |
| Gtse1 | G2 and S-phase expressed 1 | epithelium | R2M13-26 minus anti- | 1.582519 | 0.034291034 |
| | | | GFP d 5_24 h | | |
| Tdp1 | tyrosyl-DNA | epithelium | R2M13-26 minus anti- | 1.339477 | 0.034751507 |
| | phosphodiesterase 1 | | GFP d 5_24 h | | |
| Rpa2 | replication protein A2 | epithelium | R2M13-26 minus anti- | 1.05017 | 0.036291188 |
| | | | GFP d 5_24 h | | |
| Ttk | TTK protein kinase | epithelium | R2M13-26 minus anti- | 1.330748 | 0.036497959 |
| | | | GFP d 5_24 h | | |
| Timeless | timeless circadian regulator | epithelium | R2M13-26 minus anti- | 1.202788 | 0.036821283 |
| | | | GFP d 5_24 h | | |
| Ncapg | non-SMC condensin I | epithelium | R2M13-26 minus anti- | 1.114402 | 0.037236766 |
| | complex subunit G | | GFP d 5_24 h | | |
| Ncaph | non-SMC condensin I | epithelium | R2M13-26 minus anti- | 1.367556 | 0.03815944 |
| | complex subunit H | | GFP d 5_24 h | | |
| Haus1 | HAUS augmin like complex | epithelium | R2M13-26 minus anti- | 1.489593 | 0.038215468 |
| | subunit 1 | | GFP d 5_24 h | | |
| Tmem107 | transmembrane protein 107 | epithelium | R2M13-26 minus anti- | 1.113684 | 0.038332591 |
| | | | GFP d 5_24 h | | |
| Mgme1 | mitochondrial genome | epithelium | R2M13-26 minus anti- | 1.00791 | 0.039373631 |
| | maintenance exonuclease 1 | | GFP d 5_24 h | | |
| Gins2 | GINS complex subunit 2 | epithelium | R2M13-26 minus anti- | 1.185892 | 0.041317167 |
| | | | GFP d 5_24 h | | |
| Blm | BLM RecQ like helicase | epithelium | R2M13-26 minus anti- | 1.457756 | 0.042825952 |
| | | | GFP d 5_24 h | | |
| Ccna2 | cyclin A2 | epithelium | R2M13-26 minus anti- | 1.047711 | 0.042825952 |
| | | | GFP d 5_24 h | | |
| Tcf19 | transcription factor 19 | epithelium | R2M13-26 minus anti- | 1.321349 | 0.043154589 |
| | | | GFP d 5_24 h | | |
| Nusap1 | nucleolar and spindle | epithelium | R2M13-26 minus anti- | 1.15807 | 0.043742483 |
| | associated protein 1 | | GFP d 5_24 h | | |
| Ercc6l | ERCC excision repair 6 like, | epithelium | R2M13-26 minus anti- | 1.404036 | 0.045777559 |
| | spindle assembly checkpoint | | GFP d 5_24 h | | |
| | helicase | | | | |
| Pole2 | DNA polymerase epsilon 2, | epithelium | R2M13-26 minus anti- | 1.520026 | 0.046680088 |
| | accessory subunit | | GFP d 5_24 h | | |
| Nuf2 | NUF2 component of NDC80 | epithelium | R2M13-26 minus anti- | 1.158623 | 0.048977568 |
| | kinetochore complex | | GFP d 5_24 h | | |
| Tpx2 | TPX2 microtubule nucleation | epithelium | R2M13-26 minus anti- | 1.035609 | 0.049289307 |
| | factor | | GFP d 5_24 h | | |
| Paics | phosphoribosylaminoimidazole | epithelium | R2M13-26 minus anti- | 1.188391 | 0.000459648 |
| | carboxylase and | | GFP d 6_48 h | | |
| | phosphoribosylaminoimidazole- | | | | |
| | succinocarboxamide | | | | |
| | synthase | | | | |
| Knstrn | kinetochore localized astrin | epithelium | R2M13-26 minus anti- | 1.558298 | 0.001186047 |
| | (SPAG5) binding protein | | GFP d 6_48 h | | |
| Fance | FA complementation group E | epithelium | R2M13-26 minus anti- | 1.089463 | 0.01101854 |
| | | | GFP d 6_48 h | | |
| Cdkn3 | cyclin dependent kinase | epithelium | R2M13-26 minus anti- | 1.577324 | 0.014478228 |
| | inhibitor 3 | | GFP d 6_48 h | | |
| Spc24 | SPC24 component of NDC80 | epithelium | R2M13-26 minus anti- | 1.222922 | 0.018014474 |
| | kinetochore complex | | GFP d 6_48 h | | |
| Cdca8 | cell division cycle associated | epithelium | R2M13-26 minus anti- | 1.047894 | 0.018840895 |
| | 8 | | GFP d 6_48 h | | |
| Stmn1 | stathmin 1 | epithelium | R2M13-26 minus anti- | 1.063046 | 0.020701159 |
| | | | GFP d 6_48 h | | |
| Ccnb2 | cyclin B2 | epithelium | R2M13-26 minus anti- | 1.25735 | 0.020796218 |
| | | | GFP d 6_48 h | | |
| Cdk1 | cyclin dependent kinase 1 | epithelium | R2M13-26 minus anti- | 1.154977 | 0.0226473 |
| | | | GFP d 6_48 h | | |
| Gins2 | GINS complex subunit 2 | epithelium | R2M13-26 minus anti- | 1.406959 | 0.024409629 |
| | | | GFP d 6_48 h | | |
| Ccnb1 | cyclin B1 | epithelium | R2M13-26 minus anti- | 1.121675 | 0.026441863 |
| | | | GFP d 6_48 h | | |
| Poc1a | POC1 centriolar protein A | epithelium | R2M13-26 minus anti- | 1.149281 | 0.027909366 |
| | | | GFP d 6_48 h | | |
| Cdc20 | cell division cycle 20 | epithelium | R2M13-26 minus anti- | 1.069994 | 0.030688699 |
| | | | GFP d 6_48 h | | |
| Birc5 | baculoviral IAP repeat | epithelium | R2M13-26 minus anti- | 1.117698 | 0.035102419 |
| | containing 5 | | GFP d 6_48 h | | |
| Kif2c | kinesin family member 2C | epithelium | R2M13-26 minus anti- | 1.29897 | 0.040169664 |
| | | | GFP d 6_48 h | | |
| Chaf1b | chromatin assembly factor 1 | epithelium | R2M13-26 minus anti- | 1.029016 | 0.040657598 |
| | subunit B | | GFP d 6_48 h | | |
| Cdca3 | cell division cycle associated | epithelium | R2M13-26 minus anti- | 1.087222 | 0.042790541 |
| | 3 | | GFP d 6_48 h | | |
| Nup37 | nucleoporin 37 | epithelium | R2M13-26 minus anti- | 1.081951 | 0.047110197 |
| | | | GFP d 6_48 h | | |
| Cdkn3 | cyclin dependent kinase | TA2 | R2M13-26 minus anti- | 1.3120771 | 0.009450084 |
| | inhibitor 3 | | GFP | | |
| Ccnb2 | cyclin B2 | TA2 | R2M13-26 minus anti- | 1.0643399 | 0.016199086 |
| | | | GFP | | |
| Cdca8 | cell division cycle associated | TA2 | R2M13-26 minus anti- | 1.0597273 | 0.006732627 |
| | 8 | | GFP | | |
| Ccnb1 | cyclin B1 | TA2 | R2M13-26 minus anti- | 0.9711337 | 0.025176249 |
| | | | GFP | | |
| Cdc20 | cell division cycle 20 | TA2 | R2M13-26 minus anti- | 0.9321585 | 0.024278951 |
| | | | GFP | | |
| Birc5 | baculoviral IAP repeat | TA2 | R2M13-26 minus anti- | 0.9313397 | 0.010292255 |
| | containing 5 | | GFP | | |
| Hist1h1c | H1.2 linker histone, cluster | TA2 | R2M13-26 minus anti- | 0.920003 | 0.024582003 |
| | member | | GFP | | |
| Cdca3 | cell division cycle associated | TA2 | R2M13-26 minus anti- | 0.9149279 | 0.032472471 |
| | 3 | | GFP | | |
| Pbk | PDZ binding kinase | TA2 | R2M13-26 minus anti- | 0.9027055 | 0.036739905 |
| | | | GFP | | |
| Anapc15 | anaphase promoting complex | TA2 | R2M13-26 minus anti- | 0.8402356 | 0.049919161 |
| | subunit 15 | | GFP | | |
| Melk | maternal embryonic leucine | TA2 | R2M13-26 minus anti- | 0.8377668 | 0.038296186 |
| | zipper kinase | | GFP | | |
| Cdk1 | cyclin dependent kinase 1 | TA2 | R2M13-26 minus anti- | 0.8293001 | 0.044231948 |
| | | | GFP | | |
| Cenpx | centromere protein X | TA2 | R2M13-26 minus anti- | 0.816434 | 0.018966406 |
| | | | GFP | | |
| Cks2 | CDC28 protein kinase | TA2 | R2M13-26 minus anti- | 0.7936245 | 0.01843014 |
| | regulatory subunit 2 | | GFP | | |
| Spc24 | SPC24 component of NDC80 | TA2 | R2M13-26 minus anti- | 0.7683087 | 0.028434251 |
| | kinetochore complex | | GFP | | |
| Tubb4b | tubulin beta 4B class IVb | TA2 | R2M13-26 minus anti- | 0.7245988 | 0.019007425 |
| | | | GFP | | |
| Ran | RAN, member RAS | TA2 | R2M13-26 minus anti- | 0.7024503 | 0.024153629 |
| | oncogene family | | GFP | | |
| Slc29al | solute carrier family 29 | AltEnteroPC | R2M13-26 minus anti- | 2.5372566 | 0.009284855 |
| | member 1 (Augustine blood | | GFP | | |
| | group) | | | | |
| Ran | RAN, member RAS | AltEnteroPC | R2M13-26 minus anti- | 0.9721908 | 0.01482332 |
| | oncogene family | | GFP | | |
| Cdca7 | cell division cycle associated | AltEnteroPC | R2M13-26 minus anti- | 1.3346868 | 0.016301248 |
| | 7 | | GFP | | |
| Spc24 | SPC24 component of NDC80 | AltEnteroPC | R2M13-26 minus anti- | 1.5029041 | 0.02394704 |
| | kinetochore complex | | GFP | | |
| Birc5 | baculoviral IAP repeat | AltEnteroPC | R2M13-26 minus anti- | 1.2655255 | 0.024646336 |
| | containing 5 | | GFP | | |
| Dtymk | deoxythymidylate kinase | AltEnteroPC | R2M13-26 minus anti- | 1.0466804 | 0.034922079 |
| | | | GFP | | |
| Pa2g4 | proliferation-associated 2G4 | AltEnteroPC | R2M13-26 minus anti- | 0.9910247 | 0.035006869 |
| | | | GFP | | |
| Mthfd1 | methylenetetrahydrofolate | AltEnteroPC | R2M13-26 minus anti- | 0.9193116 | 0.042750243 |
| | dehydrogenase, | | GFP | | |
| | cyclohydrolase and | | | | |
| | formyltetrahydrofolate | | | | |
| | synthetase 1 | | | | |
| Stmn1 | stathmin 1 | AltEnteroPC | R2M13-26 minus anti- | 1.2770102 | 0.046544163 |
| | | | GFP | | |
| Mcm5 | minichromosome | AltEnteroPC | R2M13-26 minus anti- | 1.4828272 | 0.046544163 |
| | maintenance complex | | GFP | | |
| | component 5 | | | | |
| Mki67 | marker of proliferation Ki-67 | AltEntero1 | R2M13-26 minus anti- | 4.3362876 | 0.007949392 |
| | | | GFP | | |
| Dctpp1 | dCTP pyrophosphatase 1 | AltEntero1 | R2M13-26 minus anti- | 1.8139038 | 0.016430974 |
| | | | GFP | | |
| Birc5 | baculoviral IAP repeat | AltEntero1 | R2M13-26 minus anti- | 3.2691073 | 0.0312592 |
| | containing 5 | | GFP | | |
| Knstrn | kinetochore localized astrin | EnteroPrecur | R2M13-26 minus anti- | 3.6130018 | 0.005975418 |
| | (SPAG5) binding protein | | GFP | | |
| Stmn1 | stathmin 1 | EnteroPrecur | R2M13-26 minus anti- | 2.0258273 | 0.024502711 |
| | | | GFP | | |

TABLE 5
Illustrative anti-inflammatory genes modulated in response to Wnt agonist

| Gene | Full Gene Name | Cell Type | Condition | logFC | FDR |
|---|---|---|---|---|---|
| Gpx2 | glutathione peroxidase 2 | epithelium | R2M13-26 minus | 1.684747 | 0.002898753 |
| | | | anti-GFP d 5_24 h | | |
| Gdf15 | growth differentiation | epithelium | R2M13-26 minus | 1.329711 | 0.040472669 |
| | factor 15 | | anti-GFP d 5_24 h | | |
| Nox1 | NADPH oxidase 1 | epithelium | R2M13-26 minus | 1.519086 | 0.047660807 |
| | | | anti-GFP d 5_24 h | | |
| Gsta3 | glutathione S-transferase | epithelium | R2M13-26 minus | 2.134244 | 0.001683607 |
| | alpha 3 | | anti-GFP d 6_48 h | | |
| Gstm1 | glutathione S-transferase | epithelium | R2M13-26 minus | 1.354221 | 0.003355493 |
| | mu 1 | | anti-GFP d 6_48 h | | |
| Gpx2 | glutathione peroxidase 2 | epithelium | R2M13-26 minus | 1.266339 | 0.007234804 |
| | | | anti-GFP d 6_48 h | | |
| Gdf15 | growth differentiation | epithelium | R2M13-26 minus | 1.489524 | 0.021811964 |
| | factor 15 | | anti-GFP d 6_48 h | | |
| Sycn | syncollin | Stem cell | R2M13-26 minus | 2.1329872 | 2.70118E−10 |
| | | | anti-GFP | | |
| Il18 | interleukin 18 | Stem cell | R2M13-26 minus | 1.7187707 | 0.00015452 |
| | | | anti-GFP | | |
| Sycn | syncollin | TA1 | R2M13-26 minus | 2.4171978 | 0.02147903 |
| | | | anti-GFP | | |
| Il18 | interleukin 18 | TA2 | R2M13-26 minus | 1.7057293 | 0.006732627 |
| | | | anti-GFP | | |
| Sycn | syncollin | TA2 | R2M13-26 minus | 1.6236126 | 0.014890009 |
| | | | anti-GFP | | |
| Selenbp1 | selenium binding protein 1 | TA2 | R2M13-26 minus | 1.0085511 | 0.042565765 |
| | | | anti-GFP | | |
| Gpx2 | glutathione peroxidase 2 | TA2 | R2M13-26 minus | 0.8943478 | 0.012570697 |
| | | | anti-GFP | | |
| Tgfbr2 | transforming growth factor | AltEnteroPC | R2M13-26 minus | 1.4772761 | 0.001488099 |
| | beta receptor 2 | | anti-GFP | | |
| Gdf15 | growth differentiation | AltEnteroPC | R2M13-26 minus | 1.7517547 | 0.008596591 |
| | factor 15 | | anti-GFP | | |
| Gpx2 | glutathione peroxidase 2 | AltEnteroPC | R2M13-26 minus | 1.1711801 | 0.017581212 |
| | | | anti-GFP | | |
| Gdf15 | growth differentiation | AltEntero1 | R2M13-26 minus | 3.3934844 | 0.00000708 |
| | factor 15 | | anti-GFP | | |
| Gpx2 | glutathione peroxidase 2 | AltEntero1 | R2M13-26 minus | 1.4841473 | 0.00050619 |
| | | | anti-GFP | | |
| Tgfbr2 | transforming growth factor | AltEntero2 | R2M13-26 minus | 2.5918296 | 0.002738698 |
| | beta receptor 2 | | anti-GFP | | |
| Gdf15 | growth differentiation | EnteroPrecur | R2M13-26 minus | 3.5638037 | 0.008484168 |
| | factor 15 | | anti-GFP | | |
| Timp3 | TIMP metallopeptidase | EnteroPrecur | R2M13-26 minus | 7.4998669 | 0.020951038 |
| | inhibitor 3 | | anti-GFP | | |
| Reg4 | regenerating family | Goblet1 | R2M13-26 minus | 8.5213571 | 3.29983E−16 |
| | member 4 | | anti-GFP | | |

TABLE 6
Illustrative epithelial barrier genes modulated in response to Wnt agonist

| Gene | Full Gene Name | Cell Type | Condition | logFC | FDR |
|---|---|---|---|---|---|
| Apex1 | apurinic/apyrimidinic | epithelium | R2M13-26 minus anti- | 1.509778 | 0.00143981 |
| | endodeoxyribonuclease 1 | | GFP d 5_24 h | | |
| B3gnt7 | UDP-GlcNAc:betaGal beta-1,3-N- | Stem cell | R2M13-26 minus anti- | 7.249154 | 2.5377E−08 |
| | acetylglucosaminyltransferase 7 | | GFP | | |
| Muc3 | mucin 3A, cell surface associated | Stem cell | R2M13-26 minus anti- | 1.581388 | 3.63964E−06 |
| | | | GFP | | |
| Agr2 | anterior gradient 2, protein disulphide | TA1 | R2M13-26 minus anti- | 2.133347 | 0.02147903 |
| | isomerase family member | | GFP | | |
| Fcgbp | Fc gamma binding protein | TA1 | R2M13-26 minus anti- | 3.442198 | 0.02147903 |
| | | | GFP | | |
| Muc2 | mucin 2, oligomeric mucus/gel-forming | TA1 | R2M13-26 minus anti- | 2.820366 | 0.02147903 |
| | | | GFP | | |
| Sprr2a3 | small proline-rich protein 2A3 | TA2 | R2M13-26 minus anti- | 1.658477 | 0.007949994 |
| | | | GFP | | |
| Apex1 | apurinic/apyrimidinic | AltEnteroPC | R2M13-26 minus anti- | 1.335568 | 0.021848139 |
| | endodeoxyribonuclease 1 | | GFP | | |

TABLE 7
Wnt target genes that are modulated in the epithelial lineage as
a whole and/or in specific cell types upon R2M13-26 treatment

| Gene | Full Gene Name | Cell Type | Condition | logFC | FDR |
|---|---|---|---|---|---|
| Gsta3 | glutathione S-transferase alpha 3 | epithelium | R2M13-26 minus | 2.134244 | 0.001683607 |
| | | | anti-GFP d 6_48 h | | |
| Axin2 | axin 2 | epithelium | R2M13-26 minus | 1.768324 | 0.028289949 |
| | | | anti-GFP d 5_24 h | | |
| Myc | MYC proto-oncogene, bHLH | epithelium | R2M13-26 minus | 1.682412 | 0.005698527 |
| | transcription factor | | anti-GFP d 5_24 h | | |
| Cbr3 | carbonyl reductase 3 | epithelium | R2M13-26 minus | 1.766906 | 0.001882869 |
| | | | anti-GFP d 6_48 h | | |
| Cdkn3 | cyclin dependent kinase inhibitor 3 | epithelium | R2M13-26 minus | 1.659597 | 0.009990261 |
| | | | anti-GFP d 5_24 h | | |
| Ang | angiogenin | epithelium | R2M13-26 minus | 1.617788 | 0.014661067 |
| | | | anti-GFP d 6_48 h | | |
| Plbd1 | phospholipase B domain containing 1 | epithelium | R2M13-26 minus | 1.612844 | 0.001495964 |
| | | | anti-GFP d 6_48 h | | |
| Gtse1 | G2 and S-phase expressed 1 | epithelium | R2M13-26 minus | 1.582519 | 0.034291034 |
| | | | anti-GFP d 5_24 h | | |
| Cdkn3 | cyclin dependent kinase inhibitor 3 | epithelium | R2M13-26 minus | 1.577324 | 0.014478228 |
| | | | anti-GFP d 6_48 h | | |
| Ass1 | argininosuccinate synthase 1 | epithelium | R2M13-26 minus | 1.551104 | 0.016176013 |
| | | | anti-GFP d 6_48 h | | |
| Greb1 | growth regulating estrogen receptor | epithelium | R2M13-26 minus | 1.49355 | 0.016925608 |
| | binding 1 | | anti-GFP d 5_24 h | | |
| Aurkb | aurora kinase B | epithelium | R2M13-26 minus | 1.390542 | 0.025455962 |
| | | | anti-GFP d 5_24 h | | |
| Ncaph | non-SMC condensin I complex | epithelium | R2M13-26 minus | 1.367556 | 0.03815944 |
| | subunit H | | anti-GFP d 5_24 h | | |
| Gstm1 | glutathione S-transferase mu 1 | epithelium | R2M13-26 minus | 1.354221 | 0.003355493 |
| | | | anti-GFP d 6_48 h | | |
| Csrp2 | cysteine and glycine rich protein 2 | epithelium | R2M13-26 minus | 1.344247 | 0.006631182 |
| | | | anti-GFP d 6_48 h | | |
| Ddx39 | DExD-box helicase 39A | epithelium | R2M13-26 minus | 1.325891 | 0.009226283 |
| | | | anti-GFP d 5_24 h | | |
| Gstm3 | glutathione S-transferase mu 3 | epithelium | R2M13-26 minus | 1.32171 | 0.019200281 |
| | | | anti-GFP d 6_48 h | | |
| Cdc20 | cell division cycle 20 | epithelium | R2M13-26 minus | 1.294395 | 0.010451406 |
| | | | anti-GFP d 5_24 h | | |
| Fignl1 | fidgetin like 1 | epithelium | R2M13-26 minus | 1.279447 | 0.028531028 |
| | | | anti-GFP d 5_24 h | | |
| Prim1 | DNA primase subunit 1 | epithelium | R2M13-26 minus | 1.271932 | 0.005959274 |
| | | | anti-GFP d 5_24 h | | |
| Uhrf1 | ubiquitin like with PHD and ring | epithelium | R2M13-26 minus | 1.252776 | 0.008602422 |
| | finger domains 1 | | anti-GFP d 5_24 h | | |
| Aurka | aurora kinase A | epithelium | R2M13-26 minus | 1.251548 | 0.009399304 |
| | | | anti-GFP d 5_24 h | | |
| Hmmr | hyaluronan mediated motility receptor | epithelium | R2M13-26 minus | 1.232073 | 0.037487771 |
| | | | anti-GFP d 6_48 h | | |
| Mcm6 | minichromosome maintenance | epithelium | R2M13-26 minus | 1.23061 | 0.005959274 |
| | complex component 6 | | anti-GFP d 5_24 h | | |
| H2afz | H2A.Z variant histone 1 | epithelium | R2M13-26 minus | 1.214723 | 0.009949543 |
| | | | anti-GFP d 5_24 h | | |
| Tubb5 | tubulin beta class I | epithelium | R2M13-26 minus | 1.201435 | 0.006508255 |
| | | | anti-GFP d 5_24 h | | |
| Rnf43 | ring finger protein 43 | epithelium | R2M13-26 minus | 1.201113 | 0.008880037 |
| | | | anti-GFP d 5_24 h | | |
| Cdca3 | cell division cycle associated 3 | epithelium | R2M13-26 minus | 1.190729 | 0.027458596 |
| | | | anti-GFP d 5_24 h | | |
| Nusap1 | nucleolar and spindle associated | epithelium | R2M13-26 minus | 1.15807 | 0.043742483 |
| | protein 1 | | anti-GFP d 5_24 h | | |
| Ccnb1 | cyclin B1 | epithelium | R2M13-26 minus | 1.15428 | 0.018882603 |
| | | | anti-GFP d 5_24 h | | |
| Slc22a1 | solute carrier family 22 member 1 | epithelium | R2M13-26 minus | 1.154254 | 0.01465192 |
| | | | anti-GFP d 6_48 h | | |
| Ccnb1 | cyclin B1 | epithelium | R2M13-26 minus | 1.121675 | 0.026441863 |
| | | | anti-GFP d 6_48 h | | |
| Ncapg | non-SMC condensin I complex | epithelium | R2M13-26 minus | 1.114402 | 0.037236766 |
| | subunit G | | anti-GFP d 5_24 h | | |
| Cacybp | calcyclin binding protein | epithelium | R2M13-26 minus | 1.110488 | 0.010288843 |
| | | | anti-GFP d 5_24 h | | |
| Cdca3 | cell division cycle associated 3 | epithelium | R2M13-26 minus | 1.087222 | 0.042790541 |
| | | | anti-GFP d 6_48 h | | |
| Aifm1 | apoptosis inducing factor | epithelium | R2M13-26 minus | 1.082337 | 0.020582406 |
| | mitochondria associated 1 | | anti-GFP d 6_48 h | | |
| Abcc4 | ATP binding cassette subfamily C | epithelium | R2M13-26 minus | 1.072043 | 0.029986876 |
| | member 4 | | anti-GFP d 6_48 h | | |
| Cdc20 | cell division cycle 20 | epithelium | R2M13-26 minus | 1.069994 | 0.030688699 |
| | | | anti-GFP d 6_48 h | | |
| Adck5 | aarF domain containing kinase 5 | epithelium | R2M13-26 minus | 1.067199 | 0.012882464 |
| | | | anti-GFP d 6_48 h | | |
| Enc1 | ectodermal-neural cortex 1 | epithelium | R2M13-26 minus | 1.066644 | 0.015513811 |
| | | | anti-GFP d 5_24 h | | |
| Retsat | retinol saturase | epithelium | R2M13-26 minus | 1.052705 | 0.007480498 |
| | | | anti-GFP d 6_48 h | | |
| Gstm2 | glutathione S-transferase mu 2 | epithelium | R2M13-26 minus | 1.037239 | 0.030848582 |
| | | | anti-GFP d 6_48 h | | |
| Tpx2 | TPX2 microtubule nucleation factor | epithelium | R2M13-26 minus | 1.035609 | 0.049289307 |
| | | | anti-GFP d 5_24 h | | |
| Hsp90aa1 | heat shock protein 90 alpha family | epithelium | R2M13-26 minus | 1.02753 | 0.009990261 |
| | class A member 1 | | anti-GFP d 5_24 h | | |
| Them4 | thioesterase superfamily member 4 | epithelium | R2M13-26 minus | 1.010967 | 0.009949162 |
| | | | anti-GFP d 6_48 h | | |
| Dnajc9 | DnaJ heat shock protein family | epithelium | R2M13-26 minus | 1.010877 | 0.011604874 |
| | (Hsp40) member C9 | | anti-GFP d 5_24 h | | |
| Tbcel | tubulin folding cofactor E like | epithelium | R2M13-26 minus | −1.002073 | 0.017100865 |
| | | | anti-GFP d 5_24 h | | |
| Nuak2 | NUAK family kinase 2 | epithelium | R2M13-26 minus | −1.024343 | 0.015597046 |
| | | | anti-GFP d 6_48 h | | |
| Max | MYC associated factor X | epithelium | R2M13-26 minus | −1.05474 | 0.014043244 |
| | | | anti-GFP d 6_48 h | | |
| Endod1 | endonuclease domain containing 1 | epithelium | R2M13-26 minus | −1.097355 | 0.004966893 |
| | | | anti-GFP d 6_48 h | | |
| Prom1 | prominin 1 | epithelium | R2M13-26 minus | −1.102851 | 0.003137357 |
| | | | anti-GFP d 6_48 h | | |
| Gda | guanine deaminase | epithelium | R2M13-26 minus | −1.105871 | 0.007109783 |
| | | | anti-GFP d 6_48 h | | |
| Fgfr2 | fibroblast growth factor receptor 2 | epithelium | R2M13-26 minus | −1.117038 | 0.009891201 |
| | | | anti-GFP d 6_48 h | | |
| Srxn1 | sulfiredoxin 1 | epithelium | R2M13-26 minus | −1.148904 | 0.003836896 |
| | | | anti-GFP d 6_48 h | | |
| Slc41a2 | solute carrier family 41 member 2 | epithelium | R2M13-26 minus | −1.18125 | 0.031893846 |
| | | | anti-GFP d 6_48 h | | |
| Nav2 | neuron navigator 2 | epithelium | R2M13-26 minus | −1.204498 | 0.00436655 |
| | | | anti-GFP d 6_48 h | | |
| Iqgap2 | IQ motif containing GTPase | epithelium | R2M13-26 minus | −1.340588 | 0.007355661 |
| | activating protein 2 | | anti-GFP d 6_48 h | | |
| Dhrs9 | dehydrogenase/reductase 9 | epithelium | R2M13-26 minus | −1.522466 | 0.045290714 |
| | | | anti-GFP d 5_24 h | | |
| Xdh | xanthine dehydrogenase | epithelium | R2M13-26 minus | −1.527022 | 0.000636765 |
| | | | anti-GFP d 6_48 h | | |
| Mylk | myosin light chain kinase | epithelium | R2M13-26 minus | −1.545616 | 0.001643054 |
| | | | anti-GFP d 6_48 h | | |
| Ptpn6 | protein tyrosine phosphatase non- | epithelium | R2M13-26 minus | −1.606503 | 0.001304912 |
| | receptor type 6 | | anti-GFP d 6_48 h | | |
| Aqp8 | aquaporin 8 | epithelium | R2M13-26 minus | −1.984066 | 0.017787136 |
| | | | anti-GFP d 6_48 h | | |
| Ntrk2 | neurotrophic receptor tyrosine kinase | epithelium | R2M13-26 minus | −2.067901 | 0.040815414 |
| | 2 | | anti-GFP d 6_48 h | | |
| Adamts17 | ADAM metallopeptidase with | epithelium | R2M13-26 minus | −2.186084 | 0.035254338 |
| | thrombospondin type 1 motif 17 | | anti-GFP d 6_48 h | | |
| Rin3 | Ras and Rab interactor 3 | epithelium | R2M13-26 minus | −2.193379 | 0.020701159 |
| | | | anti-GFP d 6_48 h | | |
| Agt | angiotensinogen | epithelium | R2M13-26 minus | −2.336405 | 0.003508667 |
| | | | anti-GFP d 6_48 h | | |
| Pde4b | phosphodiesterase 4B | epithelium | R2M13-26 minus | −2.476977 | 0.016218193 |
| | | | anti-GFP d 6_48 h | | |
| Ces2a | carboxylesterase 2A | epithelium | R2M13-26 minus | −2.557209 | 0.000210476 |
| | | | anti-GFP d 6_48 h | | |
| Dhrs9 | dehydrogenase/reductase 9 | epithelium | R2M13-26 minus | −2.586128 | 0.003285788 |
| | | | anti-GFP d 6_48 h | | |
| Pdzrn3 | PDZ domain containing ring finger 3 | epithelium | R2M13-26 minus | −2.621326 | 0.010600635 |
| | | | anti-GFP d 6_48 h | | |
| Chac1 | ChaC glutathione specific gamma- | epithelium | R2M13-26 minus | −2.855996 | 0.032696198 |
| | glutamylcyclotransferase 1 | | anti-GFP d 6_48 h | | |
| Slc3a1 | solute carrier family 3 member 1 | epithelium | R2M13-26 minus | −3.071134 | 0.000312095 |
| | | | anti-GFP d 6_48 h | | |
| Cdkn1c | cyclin dependent kinase inhibitor 1C | epithelium | R2M13-26 minus | −3.285102 | 0.000856899 |
| | | | anti-GFP d 6_48 h | | |
| Tbx3 | T-box transcription factor 3 | epithelium | R2M13-26 minus | −3.463032 | 0.004904275 |
| | | | anti-GFP d 6_48 h | | |
| Ly6c1 | lymphocyte antigen 6 complex, locus | TA2 | R2M13-26 minus | 1.6287964 | 0.043275696 |
| | C1 | | anti-GFP | | |
| Cbr3 | carbonyl reductase 3 | TA2 | R2M13-26 minus | 1.5500278 | 0.026039076 |
| | | | anti-GFP | | |
| Cdkn3 | cyclin dependent kinase inhibitor 3 | TA2 | R2M13-26 minus | 1.3120771 | 0.009450084 |
| | | | anti-GFP | | |
| Aqp4 | aquaporin 4 | TA2 | R2M13-26 minus | 1.0520023 | 0.019007425 |
| | | | anti-GFP | | |
| Hmmr | hyaluronan mediated motility receptor | TA2 | R2M13-26 minus | 0.9735407 | 0.041539633 |
| | | | anti-GFP | | |
| Ccnb1 | cyclin B1 | TA2 | R2M13-26 minus | 0.9711337 | 0.025176249 |
| | | | anti-GFP | | |
| Cdc20 | cell division cycle 20 | TA2 | R2M13-26 minus | 0.9321585 | 0.024278951 |
| | | | anti-GFP | | |
| Cdca3 | cell division cycle associated 3 | TA2 | R2M13-26 minus | 0.9149279 | 0.032472471 |
| | | | anti-GFP | | |
| H2afz | H2A.Z variant histone 1 | TA2 | R2M13-26 minus | 0.8243693 | 0.008959534 |
| | | | anti-GFP | | |
| Tmem97 | transmembrane protein 97 | TA2 | R2M13-26 minus | 0.787852 | 0.033582389 |
| | | | anti-GFP | | |
| Ddx39 | DExD-box helicase 39A | TA2 | R2M13-26 minus | 0.6631842 | 0.043886108 |
| | | | anti-GFP | | |
| Slc4a4 | solute carrier family 4 member 4 | TA2 | R2M13-26 minus | −0.6817186 | 0.014890009 |
| | | | anti-GFP | | |
| Irf1 | interferon regulatory factor 1 | TA2 | R2M13-26 minus | −0.8049192 | 0.008959534 |
| | | | anti-GFP | | |
| Ets2 | ETS proto-oncogene 2, transcription | TA2 | R2M13-26 minus | −0.9529111 | 0.018546796 |
| | factor | | anti-GFP | | |
| Iffo2 | intermediate filament family orphan 2 | TA2 | R2M13-26 minus | −1.0854602 | 0.015121057 |
| | | | anti-GFP | | |
| Socs3 | suppressor of cytokine signaling 3 | TA2 | R2M13-26 minus | −1.1837287 | 0.016676011 |
| | | | anti-GFP | | |
| Cbs | cystathionine beta-synthase | TA2 | R2M13-26 minus | −1.1868333 | 0.013213267 |
| | | | anti-GFP | | |
| Rara | retinoic acid receptor alpha | TA2 | R2M13-26 minus | −1.5454715 | 0.031305634 |
| | | | anti-GFP | | |
| Ptpn6 | protein tyrosine phosphatase non- | TA2 | R2M13-26 minus | −1.5508438 | 0.042059189 |
| | receptor type 6 | | anti-GFP | | |
| Nav2 | neuron navigator 2 | TA2 | R2M13-26 minus | −1.5512663 | 0.015064266 |
| | | | anti-GFP | | |
| Per2 | period circadian regulator 2 | TA2 | R2M13-26 minus | −1.6163439 | 0.028434251 |
| | | | anti-GFP | | |
| Icam1 | intercellular adhesion molecule 1 | TA2 | R2M13-26 minus | −1.7416709 | 0.049047753 |
| | | | anti-GFP | | |
| Bcl2111 | BCL2 like 11 | TA2 | R2M13-26 minus | −1.958148 | 0.030826041 |
| | | | anti-GFP | | |
| Pim1 | Pim-1 proto-oncogene, | TA2 | R2M13-26 minus | −2.0762894 | 0.007565276 |
| | serine/threonine kinase | | anti-GFP | | |
| Pde4b | phosphodiesterase 4B | TA2 | R2M13-26 minus | −3.2363764 | 0.017171089 |
| | | | anti-GFP | | |
| Fmnl1 | formin like 1 | TA2 | R2M13-26 minus | −3.4292968 | 0.042565765 |
| | | | anti-GFP | | |
| Tgml | transglutaminase 1 | TA2 | R2M13-26 minus | −3.4897912 | 0.047774647 |
| | | | anti-GFP | | |
| Sla | Src like adaptor | TA2 | R2M13-26 minus | −3.688632 | 0.026086655 |
| | | | anti-GFP | | |
| Nlrp12 | NLR family pyrin domain containing | TA2 | R2M13-26 minus | −5.513192 | 0.032472471 |
| | 12 | | anti-GFP | | |
| Slc3a1 | solute carrier family 3 member 1 | AltEnteroPC | R2M13-26 minus | −2.0928036 | 0.001488099 |
| | | | anti-GFP | | |
| Rnf43 | ring finger protein 43 | AltEnteroPC | R2M13-26 minus | 1.5234993 | 0.004842976 |
| | | | anti-GFP | | |
| Rnase4 | ribonuclease A family member 4 | AltEnteroPC | R2M13-26 minus | 1.3663333 | 0.009933524 |
| | | | anti-GFP | | |
| Ang | Ang | AltEnteroPC | R2M13-26 minus | 1.4470403 | 0.01068326 |
| | | | anti-GFP | | |
| Pagr4 | progestin and adipoQ receptor family | AltEnteroPC | R2M13-26 minus | −0.8389525 | 0.018109402 |
| | member 4 | | anti-GFP | | |
| H2afz | H2A.Z variant histone 1 | AltEnteroPC | R2M13-26 minus | 0.9795262 | 0.020976162 |
| | | | anti-GFP | | |
| Myc | MYC proto-oncogene, bHLH | AltEnteroPC | R2M13-26 minus | 1.7404093 | 0.021596045 |
| | transcription factor | | anti-GFP | | |
| Axin2 | axin 2 | AltEnteroPC | R2M13-26 minus | 2.6986444 | 0.023278564 |
| | | | anti-GFP | | |
| Mylk | myosin light chain kinase | AltEnteroPC | R2M13-26 minus | −1.0365547 | 0.023569754 |
| | | | anti-GFP | | |
| Dhrs9 | dehydrogenase/reductase 9 | AltEnteroPC | R2M13-26 minus | −1.4543531 | 0.02409328 |
| | | | anti-GFP | | |
| Iqgap2 | IQ motif containing GTPase | AltEnteroPC | R2M13-26 minus | −0.9729629 | 0.027528807 |
| | activating protein 2 | | anti-GFP | | |
| Nap1l1 | nucleosome assembly protein 1 like 1 | AltEnteroPC | R2M13-26 minus | 0.8905891 | 0.032187986 |
| | | | anti-GFP | | |
| Tmem97 | transmembrane protein 97 | AltEnteroPC | R2M13-26 minus | 0.8972574 | 0.037953454 |
| | | | anti-GFP | | |
| Aldh3a2 | aldehyde dehydrogenase 3 family | AltEnteroPC | R2M13-26 minus | −0.8574181 | 0.038525278 |
| | member A2 | | anti-GFP | | |
| Xdh | xanthine dehydrogenase | AltEnteroPC | R2M13-26 minus | −1.1647674 | 0.046544163 |
| | | | anti-GFP | | |
| Usp18 | ubiquitin specific peptidase 18 | AltEnteroPC | R2M13-26 minus | −3.7789824 | 0.047229267 |
| | | | anti-GFP | | |
| Prom1 | prominin 1 | AltEnteroPC | R2M13-26 minus | −0.8889568 | 0.048205082 |
| | | | anti-GFP | | |
| Aqp4 | aquaporin 4 | AltEntero1 | R2M13-26 minus | −2.0782112 | 1.24E−05 |
| | | | anti-GFP | | |
| Rnase4 | ribonuclease A family member 4 | AltEntero1 | R2M13-26 minus | 2.3195336 | 0.000714494 |
| | | | anti-GFP | | |
| Tubb5 | tubulin beta class I | AltEntero1 | R2M13-26 minus | 1.9521151 | 0.005899592 |
| | | | anti-GFP | | |
| Plac8 | placenta associated 8 | AltEntero1 | R2M13-26 minus | −0.9414611 | 0.00830699 |
| | | | anti-GFP | | |
| Slc30a10 | solute carrier family 30 member 10 | AltEntero1 | R2M13-26 minus | 1.8028155 | 0.008421521 |
| | | | anti-GFP | | |
| Pim1 | Pim-1 proto-oncogene, | AltEntero1 | R2M13-26 minus | 1.7028913 | 0.015789747 |
| | serine/threonine kinase | | anti-GFP | | |
| Rnf43 | ring finger protein 43 | AltEntero1 | R2M13-26 minus | 2.1403004 | 0.032501039 |
| | | | anti-GFP | | |
| Ang | Ang | AltEntero2 | R2M13-26 minus | 2.2769739 | 0.001298915 |
| | | | anti-GFP | | |
| Rnase4 | ribonuclease A family member 4 | AltEntero2 | R2M13-26 minus | 1.6427213 | 0.013496946 |
| | | | anti-GFP | | |
| Cxcl2 | C-X-C motif chemokine ligand 2 | EnteroPrecur | R2M13-26 minus | −3.7609511 | 9.79689E−05 |
| | | | anti-GFP | | |
| Pde4b | phosphodiesterase 4B | EnteroPrecur | R2M13-26 minus | −3.7333516 | 0.002304168 |
| | | | anti-GFP | | |
| Tubb5 | tubulin beta class I | EnteroPrecur | R2M13-26 minus | 1.956738 | 0.013112905 |
| | | | anti-GFP | | |
| H2afz | H2A.Z variant histone 1 | EnteroPrecur | R2M13-26 minus | 1.2091412 | 0.024502711 |
| | | | anti-GFP | | |
| Cxcl2 | C-X-C motif chemokine ligand 2 | Enteroend2 | R2M13-26 minus | −7.9140875 | 1.61916E−21 |
| | | | anti-GFP | | |

TABLE 8
Illustrative stem and progenitor cell genes modulated in response to Wnt agonist

| Gene | Cell Type | Condition | log2FC | FDR |
|---|---|---|---|---|
| Nhp2 | epithelium | R2M13-26 minus anti-GFP | 1.857028 | 0.00143981 |
| | | d 5_24 h | | |
| Axin2 | epithelium | R2M13-26 minus anti-GFP | 1.768324 | 0.028289949 |
| | | d 5_24 h | | |
| Hmga2 | epithelium | R2M13-26 minus anti-GFP | 1.477929 | 0.000425025 |
| | | d 6_48 h | | |
| Foxq1 | epithelium | R2M13-26 minus anti-GFP | 1.520334 | 0.000861389 |
| | | d 6_48 h | | |
| Id1 | epithelium | R2M13-26 minus anti-GFP | 1.665552 | 0.001762431 |
| | | d 6_48 h | | |
| Nhp2 | epithelium | R2M13-26 minus anti-GFP | 1.189143 | 0.010340642 |
| | | d 6_48 h | | |
| Adh1 | Stem cell | R2M13-26 minus anti-GFP | 3.7215173 | 1.28645E−20 |
| Nhp2 | TA2 | R2M13-26 minus anti-GFP | 0.7075035 | 0.014352053 |
| Nhp2 | AltEnteroPC | R2M13-26 minus anti-GFP | 1.6504603 | 0.001488099 |
| Hmga2 | AltEnteroPC | R2M13-26 minus anti-GFP | 1.8393351 | 0.008885208 |
| Axin2 | AltEnteroPC | R2M13-26 minus anti-GFP | 2.6986444 | 0.023278564 |
| Foxq1 | AltEnteroPC | R2M13-26 minus anti-GFP | 1.6513882 | 0.028212078 |
| Id1 | Goblet1 | R2M13-26 minus anti-GFP | 2.7008097 | 0.000231565 |
| Areg | Goblet1 | R2M13-26 minus anti-GFP | 2.2493817 | 0.01136303 |

TABLE 9
Materials

| | | |
|---|---|---|
| Reagent or Resource | Source | Identifier |

Antibodies, Enzymatic Kits

| | | |
|---|---|---|
| Rabbit anti-Villin (SP145) | Abcam | ab130751 |
| Rabbit anti-DCLK/DCAMKL1 (D2U3L) | Cell signal | CST 62257 |
| Rabbit anti-chromogranin A | Abcam | ab15160 |
| Rabbit anti-ZO-1 (clone 1A12) | Thermofisher | 33-9100 |
| Rabbit anti-Ki67 | Abcam | 15580 |
| Rat anti-KI67 (clone SolA15) | Thermofisher | 14-5698-82 |
| Rat anti-EPCAM-Alexa-488 (clone G8.8) | Biolegend | 118210 |
| Rat anti-LY6A-Alexa-647 (clone E13-161.7) | Biolegend | 122518 |
| Rat IgG2 Isotype control-Alexa-488 | Biolegend | 400525 |
| FcR blocking Reagent | Miltenyi Biotec | 130-092-575 |
| Donkey anti-rat IgG (H&L), highly cross-adsorbed secondary | Thermofisher | A-21208 |
| antibody, Alexa Fluor 488 | | |
| Anti-GFP human IgG | Surrozen | |
| hFc-RSPO2 | Surrozen | |
| R2M3-26, bi-specific appended human IgG effector-less format | Surrozen | |
| R2M13-26, bi-specific appended human IgG effector-less format | Surrozen | |
| (parental molecule of R2M13-H26) | | |
| 1RC07-26, bi-specific appended human IgG effector-less format | Surrozen | |
| RNAscope ® 2.5 HD Assay-Red | ACD Bio | |
| RNAscope Mulitplex Fluorescent Reagent Kit, v2 Assay | ACD Bio | |
| Zymo Direct-zol RNA Microprep | Zymo | R2062 |
| MagMAX ™ mirVana ™ Total RNA Isolation Kit | Thermofisher | A27828 |
| Applied Biosystems High-Capacity cDNA Reverse Transcription | Thermofisher | 4368814 |
| Kit | | |
| Applied Biosystems TaqMan Fast Advanced Master Mix | Thermofisher | 4444557 |

Chemicals, Peptides, Proteins

| | | |
|---|---|---|
| DMEM/F12 | Thermo Fisher | 12634-010 |
| 4′,6-diamidino-2-phenylindole (DAPI) | Thermo Fisher | D1306 |
| Fetal Bovine Serum (FBS) | Thermo Fisher | 10438-026 |
| Liberase TM | Sigma | 05401127001 |
| DNAse1 | Sigma | 04716728001 |
| Ethylenediaminetetraacetic (EDTA) | | |
| Phosphate Buffered Saline (PBS) | Thermo Fisher | 10010-023 |
| HEPES | Thermo Fisher | J16924-AE |
| Sodium Pyruvate | Thermo Fisher | 11360-070 |
| Pen-Strep | Thermo Fisher | 15140-122 |
| Antibiotic/antimycotic 100X | Thermo Fisher | 15240-062 |
| Hanks Buffered Saline Solution (HBSS) | Thermo Fisher | 14175-079 |
| TrypLE | Thermo Fisher | 12604-013 |
| TX-100 | | |
| TSA Plus Cyanine 3 System | Akoya Bioscience | NEL744001KT |
| TSA Plus cyanine 5 System | Akoya Bioscience | NEL745001KT |
| Vectashield Vibrance antifade mounting medium with DAPI | Vector Laboratories | H-1800 |

TABLE 10
Basal Media Composition

| DMEM/F12K | Life technologies | | |
|---|---|---|---|
| HEPES | Life technologies | 10 | mM |
| Penicillin/streptomycin | Life technologies | 1X | |
| GlutaMAX | Life technologies | 1X | |
| N2 supplement 100x | Life technologies | 1X | |
| B27 Supplement 50x | Life technologies | 1X | |
| N-acetylcysteine | Sigma-Aldrich | 1.25 | mM |
| Recombinant human EGF | Peprotech | 50 | ng/mL |
| Recombinant human Noggin | Peprotech | 50 | ng/mL |
| Recombinant human Rspondin-1 | R&D Systems | 500 | ng/ml |

### LISTING OF REFERENCES

Aoki, R., Shoshkes-Carmel, M., Gao, N., Shin, S., May, C. L., Golson, M. L., . . . Kaestner, K. H. (2016). Foxl1-expressing mesenchymal cells constitute the intestinal stem cell niche. CMGH (New York), 2(2), 175-188. https://doi.org/10.1016/j.jcmgh.2015.12.004
Ayyaz Arshad, Kumar Sandeep, Sangiorgi Bruno, Ghoshal Bibaswan, Gosio Jessica, Ouladan Shaida, . . . Gregorieff Alex. (2019). Single-cell transcriptomes of the regenerating intestine reveal a revival stem cell. Nature, 569(7754). https://doi.org/10.1038/s41586-019-1154-y
Barker Nick, Ridgway Rachel A., van Es Johan H., van de Wetering Marc, Begthel Harry, van den Born Maaike, . . . Clevers Hans. (2009). Crypt stem cells as the cells-of-origin of intestinal cancer. Nature, 457(7229), 608-611. https://doi.org/10.1038/nature07602
Barker Nick, van Es Johan H., Kuipers Jeroen, Kujala Pekka, van den Born Maaike, Cozijnsen Miranda, . . . Clevers Hans. (2007). Identification of stem cells in small intestine and colon by marker gene Lgr5. Nature, 449(7165), 1003-1007. https://doi.org/10.1038/nature06196
Billing, L. J., Larraufie, P., Lewis, J., Leiter, A., Li, J., Lam, B., . . . Reimann, F. (2019). Single cell transcriptomic profiling of large intestinal enteroendocrine cells in mice—Identification of selective stimuli for insulin-like peptide-5 and glucagon-like peptide-1 co-expressing cells. Molecular Metabolism, 29, 158-169. https://doi.org/10.1016/j.molmet.2019.09.001
Boonekamp, Heo, Artegiani, Asra, van Son, de Ligt, & Clevers. (2021). Identification of novel human Wnt target genes using adult endodermal tissue-derived organoids. Developmental Biology, 474, 37-47. https://doi.org/10.1016/j.ydbio.2021.01.009
Bros, M., Dexheimer, N., Ross, R., Trojandt, S., Hohn, Y., Tampe, J., . . . Reske-Kunz, A. B. (2011). Differential gene expression analysis identifies murine Cacnb3 as strongly upregulated in distinct dendritic cell populations upon stimulation. Gene, 472(1-2), 18-27. https://doi.org/10.1016/j.gene.2010.10.013
Brügger, M. D., Valenta, T., Fazilaty, H., Hausmann, G., & Basler, K. (2020). Distinct populations of crypt-associated fibroblasts act as signaling hubs to control colon homeostasis. PLOS Biology, 18(12), E3001032. https://doi.org/10.1371/journal.pbio.3001032
Cabello-Aguilar, S., Alame, M., Kon-Sun-Tack, F., Fau, C., Lacroix, M., & Colinge, J. (2020). SingleCellSignalR: inference of intercellular networks from single-cell transcriptomics. Nucleic Acids Research, 48(10), E55. https://doi.org/10.1093/nar/gkaa183
Carmon, K. S., Gong, X., Lin, Q., Thomas, A., & Liu, Q. (2011). R-spondins function as ligands of the orphan receptors LGR4 and LGR5 to regulate Wnt/-catenin signaling. Proceedings of the National Academy of Sciences of the United States of America, 108(28), 11452-11457. https://doi.org/10.1073/pnas.1106083108
Chang, J. T. (2020). Pathophysiology of Inflammatory Bowel Diseases. New England Journal of Medicine, 383(27), 2652-2664. https://doi.org/10.1056/NEJMra2002697
Chateau, T., Feakins, R., Marchal-Bressenot, A., Magro, F., Danese, S., & Peyrin-Biroulet, L. (2020). Histological Remission in Ulcerative Colitis: Under the Microscope Is the Cure. American Journal of Gastroenterology, 115(2), 179-189. https://doi.org/10.14309/ajg.0000000000000437
Cheng, H., & Leblond, C. P. (1974). Origin, differentiation and renewal of the four main epithelial cell types in the mouse small intestine. V. Unitarian Theory of the origin of the four epithelial cell types. American Journal of Anatomy, 141(4), 537-561. https://doi.org/10.1002/aja.1001410407
Chen, H., Lu, C., Lee, S.-J., & Li, Y. (2020). Methods to Generate and Characterize Potent and Selective WNT Mimetic Molecules. STAR Protocols, 100043. https://doi.org/10.1016/j.xpro.2020.100043
Chen, H., Lu, C., Ouyang, B., Zhang, H., Huang, Z., Bhatia, D., . . . Li, Y. (2020). Development of Potent, Selective Surrogate WNT Molecules and Their Application in Defining Frizzled Requirements. Cell Chemical Biology, 27(5), 598-609. https://doi.org/10.1016/j.chembiol.2020.02.009
Chen, M., Reed, R. R., & Lane, A. P. (2017). Acute inflammation regulates neuroregeneration through the NF-κB pathway in olfactory epithelium. Proceedings of the National Academy of Sciences of the United States of America, 114(30), 8089-8094. https://doi.org/10.1073/pnas.1620664114
Chen, Y., Lun, A. T. L., & Smyth, G. K. (2016). From reads to genes to pathways: differential expression analysis of RNA-Seq experiments using Rsubread and the edgeR quasi-likelihood pipeline. F1000Research, 5, 1438. https://doi.org/10.12688/f1000research.8987.2
Cooper, H. S., Murthy, S. N., Shah, R. S., & Sedergran, D. J. (1993). Clinicopathologic study of dextran sulfate sodium experimental murine colitis. Laboratory Investigation, 69(2), 238-249.
Cox, C. B., Storm, E. E., Kapoor, V. N., Chavarria-Smith, J., Lin, D. L., Wang, L., . . . van Lookeren Campagne, M. (2021). IL-1R1-dependent signaling coordinates epithelial regeneration in response to intestinal damage. Science Immunology, 6(59), EABE8856. https://doi.org/10.1126/sciimmunol.abe8856
Dang, L. T., Miao, Y., Ha, A., Yuki, K., Park, K., Janda, C. Y., . . . Baker, D. (2019). Receptor subtype discrimination using extensive shape complementary designed interfaces. Nature Structural & Molecular Biology, 26(6), 407-414. https://doi.org/10.1038/s41594-019-0224-z
Degirmenci Bahar, Valenta Tomas, Dimitrieva Slavica, Hausmann George, & Basler Konrad. (2018). GLI1-expressing mesenchymal cells form the essential Wnt-secreting niche for colon stem cells. Nature, 558(7710). https://doi.org/10.1038/s41586-018-0190-3
de Lau, W. (2011). Lgr5 homologues associate with Wnt receptors and mediate R-spondin signalling. Nature, 476(7360), 57-293. https://doi.org/10.1038/NATURE10337
Eash, K. J., Greenbaum, A. M., Gopalan, P. K., & Link, D. C. (2010). CXCR2 and CXCR4 antagonistically regulate neutrophil trafficking from murine bone marrow. Journal of Clinical Investigation, The, 120(7), 2423-2431. https://doi.org/10.1172/JCI41649
Feagan, B. G., Sandborn, W. J., Lazar, A., Thakkar, R. B., Huang, B., Reilly, N., . . . Chao, J. (2014). Adalimumab Therapy Is Associated With Reduced Risk of Hospitalization in Patients With Ulcerative Colitis. Gastroenterology, 146(1), 110-118. https://doi.org/10.1053/j.gastro.2013.09.032
Flanagan, D. J., Phesse, T. J., Barker, N., Schwab, R. H. M., Amin, N., Malaterre, J., . . . Vincan, E. (2015). Frizzled7 functions as a Wnt receptor in intestinal epithelial Lgr5(+) stem cells. Stem Cell Reports, 4(5), 759-767. https://doi.org/10.1016/j.stemcr.2015.03.003
Fong, Y. W., Ho, J. J., Inouye, C., & Tjian, R. (2014). The dyskerin ribonucleoprotein complex as an OCT4/SOX2 coactivator in embryonic stem cells. ELife, 3. https://doi.org/10.7554/eLife.03573
Fowler, T. W., Mitchell, T. L., Janda, C. Y., Xie, L., Tu, S., Chen, H., . . . Li, Y. (2020). Development of selective bispecific Wnt mimetics for bone loss and repair. Nature Communications, 12(1). https://doi.org/10.1038/s41467-021-23374-8
Fowler, T. W., Mitchell, T. L., Janda, C. Y., Xie, L., Tu, S., Chen, H., . . . Li, Y. (2021). Development of selective bispecific Wnt mimetics for bone loss and repair. Nature Communications, 12(1). https://doi.org/10.1038/s41467-021-23374-8
Glinka, A., Dolde, C., Kirsch, N., Huang, Y., Kazanskaya, O., Ingelfinger, D., . . . Niehrs, C. (2011). LGR4 and LGR5 are R-spondin receptors mediating Wnt/β-catenin and Wnt/PCP signalling. EMBO Reports, 12(10), 1055-1061. https://doi.org/10.1038/embor.2011.175
Goessling, W., North, T. E., Loewer, S., Lord, A. M., Lee, S., Stoick-Cooper, C. L., . . . Zon, L. I. (2009). Genetic Interaction of PGE2 and Wnt Signaling Regulates Developmental Specification of Stem Cells and Regeneration. Cell, 136(6), 1136-1147. https://doi.org/10.1016/j.cell.2009.01.015
Goto, N., Ueo, T., Fukuda, A., Kawada, K., Sakai, Y., Miyoshi, H., . . . Seno, H. (2017). Distinct Roles of HES1 in Normal Stem Cells and Tumor Stem-like Cells of the Intestine. Cancer Research, 77(13), 3442-3454. https://doi.org/10.1158/0008-5472.CAN-16-3192
Gougelet, A., Torre, C., Veber, P., Sartor, C., Bachelot, L., Denechaud, P., . . . Colnot, S. (2014). T-cell factor 4 and β-catenin chromatin occupancies pattern zonal liver metabolism in mice. Hepatology, 59(6), 2344-2357. https://doi.org/10.1002/hep.26924
Gracz, A. D., Fuller, M. K., Wang, F., Li, L., Stelzner, M., Dunn, J. C. Y., . . . Magness, S. T. (2013). Brief report: CD24 and CD44 mark human intestinal epithelial cell populations with characteristics of active and facultative stem cells. STEM CELLS, 31(9), 2024-2030. https://doi.org/10.1002/stem.1391
Greicius, G., Kabiri, Z., Sigmundsson, K., Liang, C., Bunte, R., Singh, M. K., & Virshup, D. M. (2018). PDGFRα+ pericryptal stromal cells are the critical source of Wnts and RSPO3 for murine intestinal stem cells in vivo. Proceedings of the National Academy of Sciences of the United States of America, 115(14), E3173-E3181. https://doi.org/10.1073/pnas.1713510115
Jairath, V., & Feagan, B. G. (2020). Global burden of inflammatory bowel disease. The Lancet. Gastroenterology & Hepatology, 5(1), 2-3. https://doi.org/10.1016/S2468-1253(19)30358-9
Janda Claudia Y., Dang Luke T., You Changjiang, Chang Junlei, de Lau Wim, Zhong Zhendong A., . . . Garcia K. Christopher. (2017). Surrogate Wnt agonists that phenocopy canonical Wnt and β-catenin signalling. Nature, 545(7653), 234-237. https://doi.org/10.1038/nature22306
Janda, C. Y., Waghray, D., Levin, A. M., Thomas, C., & Garcia, K. C. (2012). Structural Basis of Wnt Recognition by Frizzled. Science, 337(6090), 59-64. https://doi.org/10.1126/science.1222879
Johansson, M. E. V., Gustafsson, J. K., Holmen-Larsson, J., Jabbar, K. S., Xia, L., Xu, H., . . . Hansson, G. C. (2014). Bacteria penetrate the normally impenetrable inner colon mucus layer in both murine colitis models and patients with ulcerative colitis. Gut, 63(2), 281-291. https://doi.org/10.1136/gutjnl-2012-303207
Jones, G.-R., Bain, C. C., Fenton, T. M., Kelly, A., Brown, S. L., Ivens, A. C., . . . MacDonald, A. S. (2018). Dynamics of Colon Monocyte and Macrophage Activation During Colitis. Frontiers in Immunology, 9, 2764. https://doi.org/10.3389/fimmu.2018.02764
Kabiri, Z., Greicius, G., Madan, B., Biechele, S., Zhong, Z., Zaribafzadeh, H., . . . Virshup, D. M. (2014). Stroma provides an intestinal stem cell niche in the absence of epithelial Wnts. Development, 141(11), 2206-2215. https://doi.org/10.1242/dev.104976
Kim, K.-A., Kakitani, M., Zhao, J., Oshima, T., Tang, T., Binnerts, M., . . . Tomizuka, K. (2005). Mitogenic Influence of Human R-Spondinl on the Intestinal Epithelium. Science, 309(5738), 1256-1259. https://doi.org/10.1126/science.1112521
Kinchen, Chen, Parikh, Antanaviciute, Jagielowicz, Fawkner-Corbett, . . . Simmons. (2018). Structural Remodeling of the Human Colonic Mesenchyme in Inflammatory Bowel Disease. Cell, 175(2), 372-386. https://doi.org/10.1016/j.cell.2018.08.067
Kipp, A., Banning, A., & Brigelius-Flohé, R. (2007). Activation of the glutathione peroxidase 2 (GPx2) promoter by β-catenin. Biological Chemistry, 388(10), 1027-1033. https://doi.org/10.1515/BC.2007.137
Kobayashi, T., Siegmund, B., Le Berre, C., & Taku, B. (2020). Ulcerative colitis. Nature Reviews Disease Primers, 6(1), 74. https://doi.org/10.1038/s41572-020-0205-x
Krausova, M., & Korinek, V. (2014). Wnt signaling in adult intestinal stem cells and cancer. Cellular Signalling, 26(3), 570-579. https://doi.org/10.1016/j.cellsig.2013.11.032
Kurahashi, M., Zheng, H., Dwyer, L., Ward, S. M., Koh, S. D., & Sanders, K. M. (2011). A functional role for the “fibroblast-like cells” in gastrointestinal smooth muscles. Journal of Physiology, The, 589(Part 3), 697-710. https://doi.org/10.1113/jphysiol.2010.201129
Kyritsis, N., Kizil, C., Zocher, S., Kroehne, V., Kaslin, J., Freudenreich, D., . . . Brand, M. (2012). Acute Inflammation Initiates the Regenerative Response in the Adult Zebrafish Brain. Science, 338(6112), 1353-1356. https://doi.org/10.1126/science.1228773
Li, B., Lee, C., Cadete, M., Miyake, H., Lee, D., & Pierro, A. (2019). Neonatal intestinal organoids as an ex vivo approach to study early intestinal epithelial disorders. Pediatric Surgery International, 35(1), 3-7. https://doi.org/10.1007/s00383-018-4369-3
Liu, K., & Nussenzweig, M. C. (2010). Origin and development of dendritic cells. Immunological Reviews, 234(1), 45-54. https://doi.org/10.1111/j.0105-2896.2009.00879.x
Lun, A. T. L., Bach, K., & Marioni, J. C. (2016). Pooling across cells to normalize single-cell RNA sequencing data with many zero counts. Genome Biology, 17, 75. https://doi.org/10.1186/s13059-016-0947-7
Mah, A. T., Yan, K. S., & Kuo, C. J. (2016). Wnt pathway regulation of intestinal stem cells. Journal of Physiology, The, 594(17), 4837-4847. https://doi.org/10.1113/JP271754
Marchal Bressenot, A., Riddell, R. H., Boulagnon-Rombi, C., Reinisch, W., Danese, S., Schreiber, S., & Peyrin-Biroulet, L. (2015). Review article: the histological assessment of disease activity in ulcerative colitis. Alimentary Pharmacology & Therapeutics, 42(8), 957-967. https://doi.org/10.1111/apt.13375
McCann, K. L., Kavari, S. L., Burkholder, A. B., Phillips, B. T., & Hall, T. M. T. (2020). H/ACA snoRNA levels are regulated during stem cell differentiation. Nucleic Acids Research, 48(15), 8686-8703. https://doi.org/10.1093/nar/gkaa612
Miao, Ha, de Lau, Yuki, Santos, You, . . . Garcia. (2020). Next-Generation Surrogate Wnts Support Organoid Growth and Deconvolute Frizzled Pleiotropy In Vivo. Cell Stem Cell, 27(5), 840-851. https://doi.org/10.1016/j.stem.2020.07.020
Muhl, L., Genové, G., Leptidis, S., Liu, J., He, L., Mocci, G., . . . Betsholtz, C. (2020). Single-cell analysis uncovers fibroblast heterogeneity and criteria for fibroblast and mural cell identification and discrimination. Nature Communications, 11(1), 3953. https://doi.org/10.1038/s41467-020-17740-1
Nile Aaron, de Sousa e Melo Felipe, Mukund Susmith, Piskol Robert, Hansen Simon, Zhou Lijuan, . . . Hannoush Rami. (2018). A selective peptide inhibitor of Frizzled 7 receptors disrupts intestinal stem cells. Nature Chemical Biology, 14(6), 582-590. https://doi.org/10.1038/s41589-018-0035-2
Nishino, J., Kim, I., Chada, K., & Morrison, S. J. (2008). Hmga2 Promotes Neural Stem Cell Self-Renewal in Young but Not Old Mice by Reducing p16\hat{ }I\hat{ }n\hat{ }k\hat{ }4\hat{ }a and p19\hat{ }A\hat{ }r\hat{ }f Expression. Cell, 135(2), 227-239. https://doi.org/10.1016/j.cell.2008.09.017
Nusse, & Clevers. (2017). Wnt/β-Catenin Signaling, Disease, and Emerging Therapeutic Modalities. Cell, 169(6), 985-999. https://doi.org/10.1016/j.cell.2017.05.016
Nusse Ysbrand, Savage Adam, Marangoni Pauline, Rosendahl-Huber Axel, Landman Tyler, de Sauvage Frederic, . . . Klein Ophir. (2018). Parasitic helminths induce fetal-like reversion in the intestinal stem cell niche. Nature, 559(7712). https://doi.org/10.1038/s41586-018-0257-1
Parisi, S., Piscitelli, S., Passaro, F., & Russo, T. (2020). HMGA Proteins in Stemness and Differentiation of Embryonic and Adult Stem Cells. International Journal of Molecular Sciences, 21(1). https://doi.org/10.3390/ijms21010362
Powell, A. E., Wang, Y., Li, Y., Poulin, E. J., Means, A. L., Washington, M. K., . . . Haigis. (2012). The Pan-ErbB Negative Regulator Lrig1 Is an Intestinal Stem Cell Marker that Functions as a Tumor Suppressor. Cell, 149(1), 146-158. https://doi.org/10.1016/j.cell.2012.02.042
Reinisch, W., Sandborn, W. J., Hommes, D. W., D'Haens, G., Hanauer, S., Schreiber, S., . . . Thakkar, R. (2011). Adalimumab for induction of clinical remission in moderately to severely active ulcerative colitis: results of a randomised controlled trial. Gut, 60(6), 780-787. https://doi.org/10.1136/gut.2010.221127
Riol-Blanco, L., S nchez-S nchez, N., Torres, A., Tejedor, A., Narumiya, S., Corb, A. L., . . . Rodr guez-Fern ndez, J. L. (2005). The Chemokine Receptor CCR7 Activates in Dendritic Cells Two Signaling Modules That Independently Regulate Chemotaxis and Migratory Speed. Journal ofImmunology, 174(7), 4070-4080. https://doi.org/10.4049/jimmunol.174.7.4070
Risso, D., Purvis, L., Fletcher, R. B., Das, D., Ngai, J., Dudoit, S., & Purdom, E. (2018). clusterExperiment and RSEC: A Bioconductor package and framework for clustering of single-cell and other large gene expression datasets. PLOS Computational Biology, 14(9), E1006378. https://doi.org/10.1371/journal.pebi.1006378
Robinson, M. D., McCarthy, D. J., & Smyth, G. K. (2010). edgeR: a Bioconductor package for differential expression analysis of digital gene expression data. Bioinformatics, (1), 139-140. https://doi.org/10.1093/bioinformatics/btp616
Rothenberg, M. E., Nusse, Y., Kalisky, T., Lee, J. J., Dalerba, P., Scheeren, F., . . . Clarke, M. F. (2012). Identification of a cKit\hat{ }+ Colonic Crypt Base Secretory Cell That Supports Lgr5\hat{ }+ Stem Cells in Mice. Gastroenterology, 142(5), 1195-1205. https://doi.org/10.1053/j.gastro.2012.02.006
Rutgeerts, P., Sandborn, W. J., Feagan, B. G., Reinisch, W., Olson, A., Johanns, J., . . . de Villiers. (2005). Infliximab for Induction and Maintenance Therapy for Ulcerative Colitis. New England Journal ofMedicine, 353(23), 2462-2476. https://doi.org/10.1056/NEJMoa050516
Sandborn, Feagan, Loftus, Peyrin-Biroulet, Van Assche, D'Haens, . . . Panes. (2020). Efficacy and Safety of Upadacitinib in a Randomized Trial of Patients With Crohn's Disease. Gastroenterology, 158(8), 2123-2138. https://doi.org/10.1053/j.gastro.2020.01.047
Sandborn, W. J., Feagan, B. G., Rutgeerts, P., Hanauer, S., Colombel, J.-F., Sands, B. E., . . . Rosari. (2013). Vedolizumab as Induction and Maintenance Therapy for Crohn's Disease. New England Journal ofMedicine, 369(8), 711-721. https://doi.org/10.1056/NEJMoa1215739
Sands, B. E., Peyrin-Biroulet, L., Loftus, E. V., Danese, S., Colombel, J.-F., Törüner, M., . . . Schreiber, S. (2019). Vedolizumab versus Adalimumab for Moderate-to-Severe Ulcerative Colitis. New England Journal of Medicine, 381(13), 1215-1226. https://doi.org/10.1056/NEJMoa1905725
Sands, B. E., Sandborn, W. J., Van Assche, G., Lukas, M., Xu, J., James, A., . . . Lasch, K. (2017). Vedolizumab as Induction and Maintenance Therapy for Crohn's Disease in Patients Naïve to or Who Have Failed Tumor Necrosis Factor Antagonist Therapy. Inflammatory Bowel Diseases, 23(1), 97-106. https://doi.org/10.1097/MIB.0000000000000979
Sasaki, N., Sachs, N., Wiebrands, K., Ellenbroek, S. I. J., Fumagalli, A., Lyubimova, A., . . . Clevers, H. (2016). Reg4+ deep crypt secretory cells function as epithelial niche for Lgr5+ stem cells in colon. Proceedings of the National Academy of Sciences of the United States of America, 113(37), E5399-E5407. https://doi.org/10.1073/pnas.1607327113
Sato Toshiro, van Es Johan H., Snippert Hugo J., Stange Daniel E., Vries Robert G., van den Born Maaike, . . . Clevers Hans. (2011). Paneth cells constitute the niche for Lgr5 stem cells in intestinal crypts. Nature, 469(7330), 415-418. https://doi.org/10.1038/nature09637
Sato Toshiro, Vries Robert G., Snippert Hugo J., van de Wetering Marc, Barker Nick, Stange Daniel E., . . . Clevers Hans. (2009). Single Lgr5 stem cells build cryptvillus structures in vitro without a mesenchymal niche. Nature, 459(7244), 262-265. https://doi.org/10.1038/nature07935
Schuijers, Junker, Mokry, Hatzis, Koo, Sasselli, . . . Clevers. (2015). Ascl2 Acts as an R-spondin/Wnt-Responsive Switch to Control Sternness in Intestinal Crypts. Cell Stem Cell, 16(2), 158-170. https://doi.org/10.1016/j.stem.2014.12.006
Schwitalla, S., Fingerle, A. A., Cammareri, P., Nebelsiek, T., Goktuna, S. I., Ziegler, P. K., . . . Schmid. (2013). Intestinal Tumorigenesis Initiated by Dedifferentiation and Acquisition of Stem-Cell-like Properties. Cell, 152(1-2), 25-38. https://doi.org/10.1016/j.cell.2012.12.012
Shoshkes-Carmel Michal, Wang Yue, Wangensteen Kirk, Tóth Beáta, Kondo Ayano, Massasa Efi, . . . Kaestner Klaus. (2018). Subepithelial telocyles are an important source of Wnts that supports intestinal crypts. Nature, 557(7704), 242-246. https://doi.org/10.1038/s41586-018-0084-4
Shvab, A., Haase, G., Ben-Shmuel, A., Gavert, N., Brabletz, T., Dedhar, S., & Ben-Ze'ev, A. (2016). Induction of the intestinal stem cell signature gene SMOC-2 is required for L1-mediated colon cancer progression. Oncogene, 35(5), 549-557. https://doi.org/10.1038/onc.2015.127
Smillie, Biton, Ordovas-Montanes, Sullivan, Burgin, Graham, . . . Regev. (2019). Intra- and Inter-cellular Rewiring of the Human Colon during Ulcerative Colitis. Cell, 178(3), 714-730. https://doi.org/10.1016/j.cell.2019.06.029
Street, K., Risso, D., Fletcher, R. B., Das, D., Ngai, J., Yosef, N., . . . Dudoit, S. (2018). Slingshot: cell lineage and pseudotime inference for single-cell transcriptomics BMC Genomics, 19(1). https://doi.org/10.1186/s12864-018-4772-0
Stzepourginski, I., Nigro, G., Jacob, J.-M., Dulauroy, S., Sansonetti, P. J., Eberl, G., & Peduto, L. (2017). CD34+ mesenchymal cells are a major component of the intestinal stem cells niche at homeostasis and after injury. Proceedings of the National Academy of Sciences of the United States of America, 114(4), E506-E513. https://doi.org/10.1073/pnas.1620059114
Tao, Y., Mis, M., Blazer, L., Ustav, M., Steinhart, Z., Chidiac, R., . . . Sidhu, S. S. (2019). Tailored tetravalent antibodies potently and specifically activate Wnt/Frizzled pathways in cells, organoids and mice ELife, 8. https://doi.org/10.7554/eLife.46134
Touw, I. P., & Beekman, R. (2013). Severe congenital neutropenia and chronic neutrophilic leukemia: an intriguing molecular connection unveiled by oncogenic mutations in CSF3R. Haematologica, 98(10), 1490-1492. https://doi.org/10.3324/haematol.2013.090571
Vanderwinden, J.-M., Rumessen, J. ri, de Kerchove d'Exaerde, A., Gillard, K., Panthier, J.-J., Laet, M.-H., & Schiffmann, S. (2002). Kit-negative fibroblast-like cells expressing SK3, a Ca2+-activated K+ channel, in the gut musculature in health and disease. Cell and Tissue Research, 310(3), 349-358. https://doi.org/10.1007/s00441-002-0638-4
van Es Johan H., Jay Philippe, Gregorieff Alex, van Gijn Marielle E., Jonkheer Suzanne, Hatzis Pantelis, . . . Clevers H. (2005). Wnt signalling induces maturation of Paneth cells in intestinal crypts. Nature Cell Biology, (4), 381-386. https://doi.org/10.1038/ncb1240
Vidal-Lletjós, S., Andriamihaja, M., Blais, A., Grauso, M., Lepage, P., Davila, A.-M., . . . Lan, A. (2019). Mucosal healing progression after acute colitis in mice. World Journal of Gastroenterology, 25(27), 3572-3589. https://doi.org/10.3748/wjg.v25.i27.3572
Wéra, O., Lancellotti, P., & Oury, C. (2016). The Dual Role of Neutrophils in Inflammatory Bowel Diseases. Journal of Clinical Medicine, 5(12). https://doi.org/10.3390/jem5120118
Whyte, J. (2012). Wnt Signaling and Injury Repair. Cold Spring Harbor Perspectives in Biology, 4(8), 133-145. https://doi.org/10.1101/CSfHPERSPECT.A008078
Wirtz Stefan, Popp Vanessa, Kindermann Markus, Gerlach Katharina, Weigmann Benno, Fichtner-Feigl Stefan, & Neurath Markus F. (2017). Chemically induced mouse models of acute and chronic intestinal inflammation. Nature Protocols, 12(7), 1295-1309. https://doi.org/10.1038/nprot.2017.044
Xu, C., & Su, Z. (2015). Identification of cell types from single-cell transcriptomes using a novel clustering method. Bioinformatics, (12), 1974-1980. https://doi.org/10.1093/bioinformatics/btv088
Yan Kelley S., Janda Claudia Y., Chang Junlei, Zheng Grace X. Y., Larkin Kathryn A., Luca Vincent C., . . . Kuo Calvin J. (2017). Non-equivalence of Wnt and R-spondin ligands during Lgr5+ intestinal stem-cell self-renewal. Nature, 545(7653), 238-242. https://doi.org/10.1038/nature22313
Yui, Azzolin, Maimets, Pedersen, Fordham, Hansen, . . . Jensen. (2018). YAP/TAZ-Dependent Reprogramming of Colonic Epithelium Links ECM Remodeling to Tissue Regeneration. Cell Stem Cell, 22(1), 35-49. https://doi.org/10.1016/j.stem.2017.11.001
Zhao, de Vera, Narushima, Beck, Palencia, Shinkawa, . . . Funk. (2007). R-spondin1, A Novel Intestinotrophic Mitogen, Ameliorates Experimental Colitis in Mice. Gastroenterology, 132(4), 1331-1343. https://doi.org/10.1053/j.gastro.2007.02.001

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications, to provide yet further embodiments. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An engineered WNT agonist comprising:
(a) one or more binding domains that bind to one or more FZD; and
(b) one or more binding domains that bind to LRP5, LRP6, or both LRP5 and LRP6,
wherein the engineered WNT agonist comprises a polypeptide sequence having at least 90%, at least 95%, at least 98%, or at least 99% sequence identity to any of SEQ ID NOs:1-25, or a polypeptide sequence disclosed in FIG. 2, FIG. 6, Table 1, or Table 3, or a binding fragment thereof; and
optionally, wherein the one or more binding domains that bind to one or more FZD bind to:
i) FZD5;
ii) FZD 8;
iii) FZD 1;
iv) FZD 2;
vi) FZD 7;
vi) FZD 5 and FZD 8;
vii) FZD 1, FZD 2, and FZD 7;
viii) FZD 1, FZD 2, FZD 7, FZD 5 and FZD 8;
ix) FZD4;
x) FZD9; or
xi) FZD10.

2. The engineered WNT agonist of claim 1, comprising:
(a) a polypeptide sequence having at least 90%, at least 95%, at least 98%, or at least 99% sequence identity to any one of SEQ ID NOs: 1-25 or a sequence disclosed in Table 3; or
(b) a polypeptide sequence comprising two or three of the CDR sequences present in any of the VHH domain, VH domain, or VL domain disclosed in FIG. 2,
optionally wherein the polypeptide sequence comprises the CDRs present in any one of SEQ ID NOs: 1-25.

3. The engineered WNT agonist of claim 2, comprising:
(a) a polypeptide sequence having least 90%, or at least 95% homology to SEQ ID NO: 1 and a polypeptide sequence having at least 90%, or at least 95% homology to SEQ ID NO:2;
(b) a polypeptide sequence having least 90%, or at least 95% homology to SEQ ID NO: 3 and a polypeptide sequence having at least 90%, or at least 95% homology to SEQ ID NO:4;
(c) a polypeptide sequence having at least 80%, at least 90%, or at least 95% homology to SEQ ID NO: 5 and a polypeptide sequence having at least 80%, at least 90%, or at least 95% homology to SEQ ID NO:6;
(d) a polypeptide sequence having at least 90%, or at least 95% homology to SEQ ID NO: 7 and a polypeptide sequence having at least 90%, or at least 95% homology to SEQ ID NO:8;
(e) a polypeptide sequence having at least 90%, or at least 95% homology to SEQ ID NO: 9 and a polypeptide sequence having at least 90%, or at least 95% homology to SEQ ID NO: 10;
(f) a polypeptide sequence having at least 90%, or at least 95% homology to SEQ ID NO: 7 and a polypeptide sequence having at least 90%, or at least 95% homology to SEQ ID NO:8
(g) a polypeptide sequence having at least 90%, or at least 95% homology to SEQ ID NO: 11 and a polypeptide sequence having at least 90%, or at least 95% homology to SEQ ID NO:12;
(h) a polypeptide sequence having at least 90%, or at least 95% homology to SEQ ID NO: 13 and a polypeptide sequence having at least 90%, or at least 95% homology to SEQ ID NO:14;
(i) a polypeptide sequence having at least 90%, or at least 95% homology to SEQ ID NO: 15 and a polypeptide sequence having at least 90%, or at least 95% homology to SEQ ID NO:16; or
(j) a polypeptide sequence having at least 90%, or at least 95% homology to SEQ ID NO: 17 and a polypeptide sequence having at least 90%, or at least 95% homology to SEQ ID NO: 18,
optionally wherein the polypeptide comprises the CDRs present in any one of SEQ ID NOs: 1-18.

4. The engineered WNT agonist of any one of claims 1-3, wherein the one or more binding domains that bind to LRP5, LRP6, or both LRP5 and LRP6 are humanized.

5. The engineered WNT agonist of any one of claims 1-4, comprising a modified Fc domain, wherein the modified Fc domain comprises a LALAPG or N297G modification.

6. A pharmaceutical composition comprising the engineered WNT agonist of any one of claims 1-5 and a pharmaceutically acceptable carrier, diluent, or excipient.

7. A method of treating a disease or disorder amenable to treatment by increased WNT pathway signaling in a subject, comprising administering to the subject the engineered WNT agonist of any one of claims 1-5 or the pharmaceutical composition of claim 6.

8. The method of claim 7, wherein the disease or disorder is a gastrointestinal disorder.

9. The method of claim 8, wherein the gastrointestinal disorder is an inflammatory bowel disease.

10. The method of claim 9, wherein the inflammatory bowel disease is selected from the group consisting of: Crohn's disease (CD), CD with fistula formation, and ulcerative colitis (UC).

11. The method of any one of claims 7-10, wherein the engineered WNT agonist is administered orally or parenterally.

12. The method of claim 11, wherein the engineered WNT agonist is administered intravenously, intraperitoneally, or subcutaneously.

13. A method of increasing WNT signaling in a cell, comprising contacting the cell with the engineered WNT agonist of any one of claims 1-5.

14. A method of modulating expression of a WNT pathway molecule in one or more tissues or cells in a subject having a gastrointestinal disorder, comprising administering to the subject the engineered WNT agonist of any one of claims 1-5 or the pharmaceutical composition of claim 6.

15. The method of claim 14, wherein the WNT pathway molecule is a gene or protein listed in any one of Tables 4, 5, 6, 7, 8, and 11.

16. The method of claim 14, wherein the WNT pathway molecule is selected from the group consisting of: glutathione peroxidase 2 (Gpx2), interferon regulatory factor 8 (Irf8), Rel, RelA, RelB, RNAse4, Angiongenin, Gsta3, Rnf43, Axin2, Ki67, Occludin, or any of the genes or proteins listed in Table 7.

17. The method of any one of claims 14-16, wherein expression of the WNT pathway molecule is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in the one or more tissues and/or cells of the subject following administration.

18. The method of any one of claims 14-17, wherein the tissue is epithelial tissue and/or the cells are gastrointestinal epithelial cells, optionally: stem cells, TA1, TA2, goblet cell progenitors, injury-induced alternative progenitors (Alt progenitors), injury-induced alternative enterocytes (Alt Enterocytes), enterocyte precursors (EnteroPrecur), goblet cell progenitors (goblet_PC), goblet cells 1, goblet cells 2, or enteroendocrine cells.

19. A method of stimulating tissue repair in a subject having a gastrointestinal disorder, comprising administering to the subject the engineered WNT agonist of any one of claims 1-5 or the pharmaceutical composition of claim 6.

20. The method of claim 19, wherein the tissue repair is stimulated by modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4, 5, 6, 7, 8, and 11.

21. The method of claim 20, wherein the genes associated with the cell cycle are selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2.

22. The method of claim 20, wherein the genes associated with stem and progenitor cell renewal and differentiation are selected from those provided in Table 8, and Axin2, Id1, Hmga2, Nhp2, Foxq1, Hes6, and Adh1.

23. The method of claim 20, wherein the genes associated with epithelial cell repair and barrier restoration are selected from those provided in Table 6, or Apex1, Agr2, B3gnt7, Fcgbp, Muc2, Muc3, Tff3, Zgl6, and Sprr2a3.

24. The method of any one of claims 20-23, wherein expression of the WNT pathway molecule is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist.

25. A method of reducing inflammation in a subject having a gastrointestinal disorder, comprising administering to the subject the engineered WNT agonist of any one of claims 1-5 or the pharmaceutical composition of claim 6.

26. The method of claim 19, wherein the inflammation is reduced by modulation of at least one molecule selected from the group consisting of: genes provided in Table 5, or Adamdec1, Atf3, Gpx2, Gsta3, Gstm1, Gstm3, Gdf15, Ihh, Il18, Lyz2, Nox1, Reg4, Sycn, Selenbp1, Tgfbr2, and Timp3.

27. The method of claim 25 or claim 26, wherein the inflammation is reduced in gastrointestinal tissue, optionally epithelial tissue.

28. The method of claim 27, wherein the gastrointestinal tissue comprises gastrointestinal epithelial cells, optionally: stem cells, TA1, TA2, goblet cell progenitors, injury-induced alternative progenitors (Alt progenitors), injury-induced alternative enterocytes (Alt Enterocytes), enterocyte precursors (EnteroPrecur), goblet cell progenitors (goblet_PC), goblet cells 1, goblet cells 2, or enteroendocrine cells.

29. The method of any one of claims 25-28, wherein expression of the WNT pathway molecule is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration.

30. The method of any one of claims 7-29, wherein the engineered Wnt agonist is R2M13-h26 or comprises a functional variant or fragment thereof.

31. A method of generating, culturing, or maintaining an organ, tissue, cell, or organoid culture, comprising contacting the organ, tissue, cell, or organoid culture with:
a) the engineered WNT agonist of any one of claims 1-5; or
b) the pharmaceutical composition of claim 6.

32. The method of claim 31 for maintaining viability of the organ or tissue ex vivo, comprising:
a) contacting an organ or tissue obtained from a donor ex vivo with a composition comprising the engineered WNT agonist or the pharmaceutical composition, optionally by perfusion; or
b) contacting a donor organ or tissue in vivo with a composition comprising the engineered WNT agonist or the pharmaceutical composition.

33. The method of claim 31 for generating or maintaining the organoid culture, comprising contacting the organoid culture, optionally by culturing the organoid culture in a medium comprising the engineered WNT agonist.

34. A method of restoring gastrointestinal epithelial barrier in a subjecting having injured epithelium, comprising administering to the subject the engineered WNT agonist of any one of claims 1-5 or the pharmaceutical composition of claim 6.

35. The method of claim 34, wherein the gastrointestinal epithelial barrier is restored by modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4, 5, 6, 7, 8, and 11.

36. The method of claim 35, wherein the genes associated with the cell cycle are selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2.

37. The method of claim 35, wherein the genes associated with stem and progenitor cell renewal and differentiation are selected from those provided in Table 8, and Axin2, Id1, Hmga2, Nhp2, Foxq1, Hes6, and Adh1.

38. The method of claim 35, wherein the genes associated with epithelial cell repair and barrier restoration are selected from those provided in Table 6, or Apex1, Agr2, B3gnt7, Fcgbp, Muc2, Muc3, Tff3, Zgl6, and Sprr2a3.

39. The method of any one of claims 35-38, wherein expression of the WNT pathway molecule is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist.

40. The method of claim 39, wherein expression of the WNT pathway molecule is increased in one or more tissues and/or cells of the subject within about 24 hours of administering the engineered Wnt agonist.

41. The method of any one of claims 34-40, wherein the subject's injured epithelium is substantially restored within about 6 days of administering the engineered Wnt agonist.

42. The method of any one of claims 34-41, wherein administration of the engineered Wnt agonist to the subject does not induce over proliferation of normal epithelium.

43. A method of inducing epithelial progenitor cell differentiation in a subject having a gastrointestinal disorder, comprising administering to the subject the engineered WNT agonist of any one of claims 1-5 or the pharmaceutical composition of claim 6.

44. The method of claim 43, wherein the epithelial cell differentiation is induced by modulation of at least one WNT pathway molecule selected from the group consisting of: genes associated with the cell cycle, genes associated with stem and progenitor cell renewal and differentiation, genes associated with epithelial cell repair and barrier restoration, and/or any of the genes listed in any of Tables 4, 5, 6, 7, 8, and 11.

45. The method of claim 44, wherein the genes associated with the cell cycle are selected from those provided in Table 4, or Aurka, Aurkb, Ccna2, Ccnb1, Ccnb2, Ccnd2, Ccne1, Cdc45, Cdk1, Cdkn3, Cenpm, Cenpp, Cenpq, Cenpu, Hells, Mcm4, Mcm5, Mcm6, Mcm7, Myc, Pbk, Plk1, Rrm1, and Rrm2.

46. The method of claim 44, wherein the genes associated with stem and progenitor cell renewal and differentiation are selected from those provided in Table 8, and Axin2, Id1, Hmga2, Nhp2, Foxq1, Hes6, and Adh1.

47. The method of claim 44, wherein the genes associated with epithelial cell repair and barrier restoration are selected from those provided in Table 6, or Apex1, Agr2, B3gnt7, Fcgbp, Muc2, Muc3, Tff3, Zgl6, and Sprr2a3.

48. The method of any one of claims 44-47, wherein expression of the WNT pathway molecule is increased by at least 20%, at least 50%, at least 80%, at least two-fold, at least five-fold, at least 10-fold, or at least 20-fold or decreased by at least 10%, 20%, 30%, 40%, 50%, 60%, 70%, 80%, or 90% in one or more tissues and/or cells of the subject following administration of the engineered Wnt agonist.

49. The method of claim 48, wherein expression of the WNT pathway molecule is increased in one or more tissues and/or cells of the subject within about 24 hours of administering the engineered Wnt agonist.

50. The method of any one of claims 43-49, wherein administration of the engineered Wnt agonist induces progenitor cell differentiation into enterocytes, goblet cells, enteroendocrine, or tuft cells in the subject.

51. The method of any one of claims 43-50, wherein substantial progenitor cell differentiation is induced in the subject within about 48 hours of administering the engineered Wnt agonist.

52. The method of any one of claims 43-51, wherein administration of the engineered Wnt agonist to the subject does not induce over proliferation of normal epithelium.
